(19) United States
(12) Patent Application Publication
(10) Pub. No.: US 2024/0044872 A1
(43) Pub. Date: Feb. 8, 2024

(54) METHOD FOR ASSESSING THE OPSONOPHAGOCYTOTIC CAPACITY OR TROGOCYTOTIC CAPACITY OF AN ANTIGEN-BINDING MOLECULE

(71) Applicant: 10x Genomics, Inc., Pleasanton, CA (US)

(72) Inventor: Wyatt James MCDONNELL, Pleasanton, CA (US)

(21) Appl. No.: 18/448,553

(22) Filed: Aug. 11, 2023

Related U.S. Application Data
(63) Continuation of application No. PCT/US22/16222, filed on Feb. 11, 2022.
(60) Provisional application No. 63/148,944, filed on Feb. 12, 2021.

(51) Int. Cl. G01N 33/50; G01N 33/563

(52) U.S. Cl. CPC G01N 33/5047; G01N 33/563; G01N 2333/705

(57) ABSTRACT

The present disclosure relates to a method for identifying opsonophagocytotic activity and/or trogocytotic activity of an antigen-binding molecule.

### CROSS-REFERENCE TO RELATED APPLICATIONS

This application claims the benefit of U.S. Provisional Patent Application No. 63/148,944, filed Feb. 12, 2021, which is incorporated herein by reference in its entirety.

### FIELD

The present disclosure relates to a method for identifying opsonophagocytotic activity and/or trogocytotic activity of an antigen-binding molecule.

### BACKGROUND

Antibodies are biomolecules produced by adaptive B-lymphocytes, with exquisite specificity and affinity to their target(s). The function of antibodies can differ significantly depending on the effector activity of the antibody, with functions including direct neutralization or ligand blocking, antibody-induced cytotoxicity, antibody-induced complement deposition, and antibody-induced opsonophagocytosis. In the latter case, IgG antibodies can enable deposition of complement proteins onto their targets and encouraging the immune system to ingest and destroy the antibody-antigen complex. This is an ideal property for therapeutic antibodies and antibodies elicited by successful vaccines to possess.

Traditional assays to assess opsonophagocytotic or trogocytotic activity of an antibody are limited in their throughput and scale. Here, by utilizing oligonucleotide barcoding, we describe a new opsonophagocytotic assay with modifications that enable both the detection of deposited complement on a target antigen and the detection of phagocytosed antibody and antigen. This assay is also able to detect trogocytosis, where an antibody can extract an antigen from a target cell.

### SUMMARY

The present disclosure provides, among others, a method for identifying opsonophagocytotic activity and/or trogocytotic activity of an antigen-binding molecule. In some embodiments, the method comprises: a) contacting an antigen with a composition comprising an antigen-binding molecule to create a complex comprising the antigen bound to the antigen-binding molecule, wherein said antigen-binding molecule comprises a first oligonucleotide comprising a first barcode sequence; b) contacting the complex from with a plurality of immune effector cells under conditions sufficient to provide a first immune effector cell comprising the complex as a phagocytosed complex; c) partitioning the plurality of immune effector cells into a plurality of partitions, wherein a partition of said plurality of partitions comprises (i) the first immune effector cell and (ii) a plurality of nucleic acid barcode molecules wherein a first nucleic acid barcode molecule of the plurality of nucleic acid barcode molecules comprises a partition barcode sequence; d) in the partition, coupling the first oligonucleotide to the first nucleic acid barcode molecule; and e) using the first oligonucleotide coupled to the first nucleic acid barcode molecule to generate a first barcoded nucleic acid molecule comprising the first barcode sequence or a complement thereof and the partition barcode sequence or a complement thereof.

The present disclosure also provides a method for identifying opsonophagocytotic activity and/or trogocytotic activity of an antigen-binding molecule, comprising: (a) contacting an antigen with an antigen-binding molecule to create a complex comprising the antigen bound to the antigen-binding molecule, wherein the antigen-binding molecule comprises a first oligonucleotide comprising a first barcode sequence; (b) contacting the complex with a plurality of immune effector cells under conditions sufficient to provide a first immune effector cell comprising the complex as a phagocytosed complex; (c) partitioning the plurality of immune effector cells into a plurality of partitions, wherein a partition of the plurality of partitions comprises (i) the first immune effector cell and (ii) a plurality of nucleic acid barcode molecules wherein a first nucleic acid barcode molecule of the plurality of nucleic acid barcode molecules comprises a partition barcode sequence; and (d) in the partition, using the first oligonucleotide and the first nucleic acid barcode molecule to generate a first barcoded nucleic acid molecule comprising the first barcode sequence or a complement thereof and the partition barcode sequence or a complement thereof.

The present disclosure also provides a method for identifying opsonophagocytotic activity and/or trogocytotic activity of an antigen-binding molecule. In some embodiments, the method comprises: a) contacting an antigen with a composition comprising an antigen-binding molecule to create a complex comprising the antigen bound to the antigen-binding molecule, wherein said antigen-binding molecule comprises a first oligonucleotide comprising a first barcode sequence; b) contacting the complex from (a) with a plurality of immune effector cells under conditions sufficient to provide a first immune effector cell comprising said complex as a phagocytosed complex; c) partitioning the plurality of immune effector cells from (b) into a plurality of partitions, wherein a partition of said plurality of partitions comprises (i) said first immune effector cell and (ii) a plurality of nucleic acid barcode molecules; and d) in the partition, generating a plurality of barcoded nucleic acid molecules that comprises the first barcode sequence or a complement thereof, which identifies said antigen-binding molecule as having opsonophagocytotic activity and/or trogocytotic activity.

In some embodiments of any of the foregoing methods, the antigen comprises a second oligonucleotide comprising a second barcode sequence. In some embodiments, the plurality of barcoded nucleic acid molecules further comprise the second barcode sequence or a complement thereof. In some embodiments, the second nucleic acid barcode molecule of the plurality of nucleic acid barcode molecules comprises the partition barcode sequence, and the method further comprises using the second oligonucleotide and the second nucleic acid barcode molecule to generate a second barcoded nucleic acid molecule comprising the second barcode sequence or a reverse complement thereof and the partition barcode sequence or a reverse complement thereof.

In some embodiments of any of the foregoing methods, the contacting in (b) further comprises conditions sufficient to allow opsonization of said antigen. In some embodiments, the opsonization of said antigen comprises opsonin deposition of said antigen. In some embodiments, the opsonin deposition of said antigen comprises complement deposition of said antigen.

In some embodiments of any of the foregoing methods, the method further comprises contacting the plurality of immune effector cells with an anti-opsonin antibody. In some embodiments, the anti-opsonin antibody is an anti-complement antibody. In some embodiments, the anti-opsonin antibody comprises a third oligonucleotide comprising a third barcode sequence. In some embodiments, the plurality of barcoded nucleic acid molecules further comprises the third barcode sequence or a complement thereof. In some embodiments, the third nucleic acid barcode molecule of the plurality of nucleic acid barcode molecules comprises the partition barcode sequence, and the method further comprises using the third oligonucleotide and the third nucleic acid barcode molecule to generate a third barcoded nucleic acid molecule comprising the third barcode sequence or a reverse complement thereof and the partition barcode sequence or a reverse complement thereof.

In some embodiments of any of the foregoing methods, the method further comprises contacting the plurality of immune effector cells with other binding agents to complement or opsonin. In some embodiments, these agents include, complement family members (e.g., Factor H, C1q) that are barcoded and anti-glycan molecules.

In some embodiments of any of the foregoing methods, the immune effector cell comprises a nucleic acid analyte, and a fourth nucleic acid barcode molecule of the plurality of nucleic acid barcode molecules comprises the partition barcode sequence, and the method further comprises using the nucleic acid analyte and the fourth nucleic acid barcode molecule to generate a fourth barcoded nucleic acid molecule comprising a sequence of the nucleic acid analyte or a reverse complement thereof and the partition barcode sequence or a reverse complement thereof.

In certain embodiments of any of the foregoing methods, the antigen is presented on the surface of an antigen-presenting cell (APC). In some embodiments of any of the foregoing methods, the antigen is conjugated to a support. In some embodiments, the support comprises a bead. In certain embodiments, the bead comprises gel beads, glass beads, magnetic beads, and/or ceramic beads.

In some embodiments of any of the foregoing methods, the plurality of nucleic acid barcode molecules comprises a partition barcode sequence.

In some embodiments of any of the foregoing methods, the plurality of immune effector cells is (i) capable of mediating antibody-dependent cellular phagocytosis (ADCP) and/or (ii) capable of antibody-dependent cellular trogocytosis (ADCT). In some embodiments, the plurality of immune effector cells comprises a plurality of phagocytotic cells and/or a plurality of trogocytotic cells. In some embodiments, the plurality of phagocytic cells comprises a plurality of neutrophils, monocytes, macrophages, mast cells, and/or dendritic cells. In some embodiments, the plurality of trogocytotic cells comprises a plurality of B cells, T cells, monocytes, neutrophils, and/or natural killer (NK) cells.

In some embodiments of any of the foregoing methods, the method further comprises separating the first immune effector cell from a second immune effector cell which does not comprise a phagocytosed complex. In some embodiments, the method further comprises separating the first immune effector cell from a second immune effector cell which does not comprise a phagocytosed complex via said support. In some embodiments, the support allows for said separating step using (i) a density difference between the first immune effector cell and the second immune effector cell or (ii) a magnetic difference between the first immune effector cell and the second immune effector cell. In some embodiments, the separating step is prior to said partitioning step.

In some embodiments of any of the foregoing methods, the method further comprises sorting said plurality of immune effector cells prior to said partitioning step. In some embodiments, the sorting is via a label. In some embodiments, the one or more of the support, the APC, the anti-complement antibody, the anti-opsonin antibody, the antigen, the antigen-binding molecule, and/or the plurality of immune effector cells further comprises said label. In some embodiments, the label comprises a fluorophore label, a colorimetric label, a magnetic label, and/or a sortable antibody label. In some embodiments, the sortable antibody label is conjugated to a barcode molecule.

In some embodiments of any of the foregoing methods, the plurality of barcoded nucleic acid molecules comprises a first barcoded nucleic acid molecule comprising said first barcode sequence or a complement thereof and said partition barcode sequence or a complement thereof. In some embodiments, the plurality of nucleic acid barcode molecules comprises a partition barcode sequence. In some embodiments, the plurality of barcoded nucleic acid molecules comprises a second barcoded nucleic acid molecule comprising said second barcode sequence or a complement thereof and said partition barcode sequence or a complement thereof. In some embodiments, the plurality of barcoded nucleic acid molecules comprises a third barcoded nucleic acid molecule comprising said third barcode sequence or a complement thereof and said partition barcode sequence or a complement thereof. In some embodiments, the plurality of barcoded nucleic acid molecules comprise an additional barcoded nucleic acid molecule comprising a sequence corresponding to a messenger ribonucleic acid (mRNA) molecule encoding for an immune receptor from said first immune effector cell.

In some embodiments of any of the foregoing methods, the method further comprises comparing the number of partitioned immune effector cells that have ingested the complex and/or at least one complement components to a reference number quantified for a plurality of reference cells. In some embodiments, the method further comprises comparing the percentage of partitioned immune effector cells that have ingested the complex and/or the at least one complement components to a reference percentage quantified for the plurality of reference effector cells. In some embodiments, the plurality of reference effector cells has been contacted with the complex comprising the antigen bound to the antigen-binding molecule, and wherein the plurality of reference effector cells have been further contacted with an Fc blocking reagent. In some embodiments, the plurality of reference effector cells have been contacted with (i) an antigen coated with neutravidin, (ii) a negative control having or suspected of having little or no opsonophagocytotic or trogocytotic effects, or (iii) a positive control antibody having or suspected of having opsonophagocytotic or trogocytotic effects.

In some embodiments, the antigen is conjugated to a partition-specific barcode molecule. In some embodiments, the partition-specific barcode molecule comprises one or more of the following: a peptide tag, an oligonucleotide barcode, a functional sequence, a common barcode, a UNIT, and a reporter capture sequence. In some embodiments, the first, second, third, and/or fourth nucleic acid barcode molecule of the plurality of nucleic acid barcode molecules comprises one or more of the following: a functional sequence, and a UNIT sequence, optionally wherein the first nucleic acid barcode molecule further comprises a first capture sequence configured to couple to the first oligonucleotide, and/or the second nucleic acid barcode molecule further comprises a second capture sequence configured to couple to the second oligonucleotide, and/or the third nucleic acid barcode molecule further comprises a third capture sequence configured to couple to the second oligonucleotide, and/or the fourth nucleic acid barcode molecule further comprises a fourth capture sequence, wherein the fourth capture sequence is configured to couple to a sequence of the nucleic acid analyte or is a template switch oligonucleotide. In some embodiments, the antigen-binding molecule is conjugated to a reporter oligonucleotide. In certain embodiments, the reporter oligonucleotide comprises one or more of the following: a reporter capture handle, a reporter sequence, and/or a functional sequence. In certain embodiments, the reporter capture handle comprising a sequence that is complementary to the reporter capture sequence.

In some embodiments of any of the foregoing methods, the method further comprises determining a sequence of the first barcoded nucleic acid molecule or a derivative thereof, the second barcoded nucleic acid molecule or a derivative thereof, the third barcoded nucleic acid molecule or a derivative thereof, and/or the fourth barcoded nucleic acid molecule or a derivative thereof. In some embodiments, the method comprises (i) using the determined sequence of the first barcoded nucleic acid molecule or a derivative thereof to identify the antigen binding molecule as having been opsonophagocytosed and/or trogocytosed by the first immune effector cell, (ii) using the determined sequence of the second barcoded nucleic acid molecule or a derivative thereof to identify the antigen binding molecule as having bound the antigen, and/or (iii) using the determined sequence of the third barcoded nucleic acid molecule or a derivative thereof to identify the antigen as having been opsonized.

The present disclosure also provides a composition, comprising an immune effector cell associated with a complex, the complex comprising an antigen-binding molecule bound to an antigen, wherein the antigen-binding molecule (i) is exogenous to the immune effector cell and (ii) comprises a first oligonucleotide comprising a first barcode sequence. In some embodiments, the complex is a phagocytosed complex within the immune effector cell, and/or the antigen comprises a second oligonucleotide comprising a second barcode sequence, and/or the antigen is associated with opsonin deposition, optionally wherein the opsonin deposition comprises complement deposition, and/or the antigen is conjugated to a support, optionally wherein the support comprises a bead, optionally wherein the bead comprises gel beads, glass beads, magnetic beads, and/or ceramic beads. In some embodiments, the composition further comprises a partition comprising the immune effector cell, optionally wherein the partition is a droplet or a well, and/or the partition further comprises a plurality of nucleic acid barcode molecules, wherein a first nucleic acid barcode molecule of the plurality of nucleic acid barcode molecules comprises a partition barcode sequence, optionally wherein the plurality of nucleic acid barcode molecules are attached to a bead, optionally wherein the bead is a solid bead, a magnetic bead, or a gel bead. In some embodiments, the immune effector cell of the composition is (i) capable of mediating antibody-dependent cellular phagocytosis (ADCP) and/or (ii) capable of antibody-dependent cellular trogocytosis (ADCT), and/or (iii) a phagocytic cell and/or a trogocytotic cell, optionally wherein the phagocytic cell is selected from a neutrophil, monocyte, macrophage, mast cell, and dendritic cell, optionally wherein the trogocytotic cell is selected from a B cell, T cell, monocyte, neutrophil, and natural killer cell.

The present disclosure also provides a system, comprising a) an antigen binding molecule comprising a first oligonucleotide comprising a first barcode sequence and b) an antigen that binds the antigen binding molecule; and a plurality of nucleic acid barcode molecules, wherein a first nucleic acid barcode molecule of the plurality of nucleic acid barcode molecules comprises a partition barcode sequence. In some embodiments, the plurality of nucleic acid barcode molecules is attached to a bead, and the partition barcode sequence identifies the bead. In some embodiments, the first nucleic acid barcode molecule comprises a first capture sequence configured to couple to the first oligonucleotide. In some embodiments, the first oligonucleotide further comprises a capture handle sequence configured to couple to the capture sequence of the first nucleic acid barcode molecule. In some embodiments, the antigen comprises a second oligonucleotide comprising a second barcode sequence. In some embodiments, a second nucleic acid barcode molecule of the plurality of nucleic acid barcode molecules comprises the partition barcode sequence and a second capture sequence configured to couple to the second oligonucleotide. In some embodiments, (i) the first capture sequence and the second capture sequence are identical, or (ii) the first capture sequence and the second capture sequence are different.

In some embodiments, the system further comprises an anti-opsonin antibody comprising a third oligonucleotide comprising a third barcode sequence. In some embodiments, a third nucleic acid barcode molecule of the plurality of nucleic acid barcode molecules comprises the partition barcode sequence and a third capture sequence configured to couple to the second oligonucleotide. In some embodiments, a fourth nucleic acid barcode molecule of the plurality of nucleic acid barcode molecules comprises the partition barcode sequence and a fourth capture sequence, wherein the fourth capture sequence is configured to couple to a sequence of the nucleic acid analyte or is a template switch oligonucleotide.

In some embodiments, the system further comprises a plurality of partitions, optionally wherein the plurality of partitions comprises a plurality of droplets and/or a plurality of wells.

In some embodiments, the system further comprises an apparatus comprising a microfluidic channel structure configured to generate a plurality of partitions.

The present disclosure also provides a kit comprising a) reagents configured to conjugate a first oligonucleotide comprising a first barcode sequence to an antigen binding molecule, and b) instructions for performing a method of any one of the preceding claims. In some embodiments, the kit further comprises the first oligonucleotide. In some embodiments, the reagents are configured to conjugate a second oligonucleotide comprising a second barcode sequence to an antigen capable of binding the antigen binding molecule, and the kit further comprises the second oligonucleotide. In some embodiments, the reagents are configured to conjugate a third oligonucleotide comprising a third barcode sequence to an anti-opsonin antibody, and the kit further comprises the third oligonucleotide.

In some embodiments, the kit further comprises an anti-opsonin antibody comprising a third oligonucleotide that comprises a third barcode sequence.

In some embodiments, the kit further comprises a support, where the reagents are configured to conjugate the antigen to the support, or where the kit further comprises reagents configured to conjugate the antigen to the support.

In some embodiments, the kit further comprises a control antigen that is configured to or expected to not bind the antigen binding molecule.

In some embodiments, the kit further comprises a population of immune effector cells.

### BRIEF DESCRIPTION OF THE DRAWINGS

FIG. 1 shows an exemplary microfluidic channel structure for partitioning individual biological particles in accordance with some embodiments of the disclosure.

FIG. 2 shows an exemplary microfluidic channel structure for the controlled partitioning of beads into discrete droplets.

FIG. 3 shows an exemplary barcode carrying bead.

FIG. 4 illustrates another example of a barcode carrying bead.

FIG. 5 schematically illustrates an example microwell array.

FIG. 6 schematically illustrates an example workflow for processing nucleic acid molecules.

FIG. 7 schematically illustrates examples of labelling agents.

FIG. 8 depicts an example of a barcode carrying bead.

FIGS. 9A-9C schematically depict an example workflow for processing nucleic acid molecules.

FIGS. 10A-10C illustrate examples of antibody-dependent cellular phagocytosis and antibody-dependent cellular trogocytosis workflow.

FIGS. 11A-11D illustrate examples of opsonin-mediated phagocytosis workflow.

FIG. 12 shows an example of a microfluidic channel structure for delivering barcode carrying beads to droplets.

### DETAILED DESCRIPTION OF THE DISCLOSURE

The present disclosure relates to a method for identifying opsonophagocytotic activity and/or trogocytotic activity of an antigen-binding molecule. In brief, the method includes contacting an antigen with a composition comprising an antigen-binding molecule to create a complex. The complex thus contains the antigen bound to the antigen-binding molecule. In some embodiments, the antigen-binding molecule comprises a first oligonucleotide comprising a first barcode sequence. In addition, the method further includes contacting the complex described immediately above with a plurality of immune effector cells under conditions sufficient to provide a first immune effector cell comprising said complex as a phagocytosed complex, partitioning the plurality of immune effector cells into a plurality of partitions. In some embodiments, a partition of the plurality of partitions comprises (i) the first immune effector cell and (ii) a plurality of nucleic acid barcode molecules. The method also includes, in the partition, generating a plurality of barcoded nucleic acid molecules that comprises the first barcode sequence or a complement thereof, which identifies the antigen-binding molecule as having opsonophagocytotic activity and/or trogocytotic activity.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

An “adapter,” an “adaptor,” and a “tag” are terms that are used interchangeably in this disclosure, and refer to moieties that can be coupled to a polynucleotide sequence (in a process referred to as “tagging”) using any one of many different techniques including (but not limited to) ligation, hybridization, and tagmentation. Adapters can also be nucleic acid sequences that add a function, e.g., spacer sequences, primer sequences, primer binding sites, barcode sequences, and unique molecular identifier sequences.

The term “barcode” is used herein to refer to a label, or identifier, that conveys or is capable of conveying information (e.g., information about an analyte in a sample, a bead, and/or a nucleic acid barcode molecule). A barcode can be part of an analyte or nucleic acid barcode molecule, or independent of an analyte or nucleic acid barcode molecule. A barcode can be attached to an analyte or nucleic acid barcode molecule in a reversible or irreversible manner. A particular barcode can be unique relative to other barcodes. Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for or facilitates identification and/or quantification of individual sequencing-reads. In some embodiments, a barcode can be configured for use as a fluorescent barcode. For example, in some embodiments, a barcode can be configured for hybridization to fluorescently labeled oligonucleotide probes. Barcodes can be configured to spatially resolve molecular components found in biological samples, for example, at single-cell resolution (e.g., a barcode can be or can include a “spatial barcode”). In some embodiments, a barcode includes two or more sub-barcodes that together function as a single barcode. For example, a polynucleotide barcode can include two or more polynucleotide sequences (e.g., sub-barcodes). In some embodiments, the two or more sub-barcodes are separated by one or more non-barcode sequences. In some embodiments, the two or more sub-barcodes are not separated by non-barcode sequences.

As used herein, a complement of a barcode sequence refers to a nucleic acid sequence that is complementary to the barcode sequence. The term “complementary” is used as its common meaning in the art and refers to the natural binding of polynucleotides by base pairing. The complementarity of two polynucleotide strands is achieved by distinct interactions between nucleobases: adenine (A), thymine (T) (uracil (U) in RNA), guanine (G), and cytosine (C). Adenine and guanine are purines, while thymine, cytosine, and uracil are pyrimidines. Both types of molecules complement each other and can only base pair with the opposing type of nucleobase by hydrogen bonding. For example, an adenine can only be efficiently paired with a thymine (A=T) or a uracil (A=U), and a guanine can only be efficiently paired with a cytosine (G≡C). The base complement A=T or A=U shares two hydrogen bonds, while the base pair G≡C shares three hydrogen bonds. The two complimentary strands are oriented in opposite directions, and they are said to be antiparallel. For another example, the sequence 5′-A-G-T 3′ binds to the complementary sequence 3′-T-C-A-5′. The degree of complementarity between two strands may vary from complete (or perfect) complementarity to no complementarity. The degree of complementarity between polynucleotide strands has significant effects on the efficiency and strength of the hybridization between the nucleic acid strands. In some embodiments, the complement of a barcode sequence provided herein is perfectly complimentary to the barcode sequence.

In some embodiments, a barcode can include one or more unique molecular identifiers (UMIs). Generally, a unique molecular identifier is a contiguous nucleic acid segment or two or more non-contiguous nucleic acid segments that function as a label or identifier for a particular analyte, or for a nucleic acid barcode molecule that binds a particular analyte (e.g., mRNA) via the capture sequence.

A UMI can include one or more specific polynucleotides sequences, one or more random nucleic acid and/or amino acid sequences, and/or one or more synthetic nucleic acid and/or amino acid sequences. In some embodiments, the UMI is a nucleic acid sequence that does not substantially hybridize to analyte nucleic acid molecules in a biological sample. In some embodiments, the UNIT has less than 80% sequence identity (e.g., less than 70%, 60%, 50%, or less than 40% sequence identity) to the nucleic acid sequences across a substantial part (e.g., 80% or more) of the nucleic acid molecules in the biological sample. These nucleotides can be completely contiguous, i.e., in a single stretch of adjacent nucleotides, or they can be separated into two or more separate subsequences that are separated by 1 or more nucleotides.

The terms “cell”, “cell culture”, “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the originally cell, cell culture, or cell line.

As used herein, the term “functional fragment thereof” or “functional variant thereof” relates to a molecule having qualitative biological activity in common with the wild-type molecule from which the fragment or variant was derived. For example, a functional fragment or a functional variant of an antibody is one which retains essentially the same ability to bind to the same epitope as the antibody from which the functional fragment or functional variant was derived. A number of methods known in the field can be suitably used to test the functionality or activity of a compound, e.g. peptide or protein. In some embodiments, the functional variant of the encoded wild-type protein can also include any fragment of the wild-type protein or fragment of a modified protein that has conservative modification on one or more of amino acid residues in the corresponding full length, wild-type protein. In some embodiments, the functional variant of the encoded wild-type protein can also include any modification(s), e.g. deletion, insertion and/or mutation of one or more amino acids that do not substantially negatively affect the functionality of the wild-type protein.

As used herein, a “subject” or an “individual” includes animals, such as human (e.g., human individuals) and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

The term “biological particle” is used herein to generally refer to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix. The term “macromolecular constituent,” as used herein, generally refers to a macromolecule contained within or derived from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value±up to 10%, up to ±5%, or up to ±1%.

The term “microwell,” as used herein, generally refers to a well with a volume of less than 1 mL. Microwells may be made in various volumes, depending on the application. For example, microwells may be made in a size appropriate to accommodate any of the partition volumes described herein.

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

Headings, e.g., (a), (b), (i) etc., are presented merely for ease of reading the specification and claims. The use of headings in the specification or claims does not require the steps or elements be performed in alphabetical or numerical order or the order in which they are presented.

Use of ordinal terms such as “first”, “second”, “third”, “fourth”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each combination was individually and explicitly disclosed. In addition, all sub-combinations of the various embodiments and elements thereof are also specifically embraced by the present disclosure and are disclosed herein just as if each such sub-combination was individually and explicitly disclosed herein.

Method for Identifying Opsonophagocytotic Activity and/or Trogocytotic Activity of an Antigen-Binding Molecule

One aspect of the present disclosure relates to a method for identifying opsonophagocytotic activity and/or trogocytotic activity of an antigen-binding molecule.

The method for identifying opsonophagocytotic activity and/or trogocytotic activity of an antigen-binding molecule as provided herein includes contacting an antigen with a composition comprising an antigen-binding molecule to create a complex comprising the antigen bound to the antigen-binding molecule.

In some embodiments, the method comprises: a) contacting an antigen with a composition comprising an antigen-binding molecule to create a complex comprising the antigen bound to the antigen-binding molecule, wherein said antigen-binding molecule comprises a first oligonucleotide (e.g., first reporter oligonucleotide) comprising a first barcode sequence (e.g., first reporter sequence); b) contacting the complex from with a plurality of immune effector cells under conditions sufficient to provide a first immune effector cell comprising the complex as a phagocytosed complex; c) partitioning the plurality of immune effector cells into a plurality of partitions, wherein a partition of said plurality of partitions comprises (i) the first immune effector cell and (ii) a plurality of nucleic acid barcode molecules wherein a first nucleic acid barcode molecule of the plurality of nucleic acid barcode molecules comprises a partition barcode sequence; d) in the partition, coupling the first oligonucleotide to the first nucleic acid barcode molecule; and e) using the first oligonucleotide coupled to the first nucleic acid barcode molecule to generate a first barcoded nucleic acid molecule comprising the first barcode sequence or a complement thereof and the partition barcode sequence or a complement thereof.

In some embodiments, the method comprises: (a) contacting an antigen with an antigen-binding molecule to create a complex comprising the antigen bound to the antigen-binding molecule, wherein the antigen-binding molecule comprises a first oligonucleotide comprising a first barcode sequence; (b) contacting the complex with a plurality of immune effector cells under conditions sufficient to provide a first immune effector cell comprising the complex as a phagocytosed complex; (c) partitioning the plurality of immune effector cells into a plurality of partitions, wherein a partition of the plurality of partitions comprises (i) the first immune effector cell and (ii) a plurality of nucleic acid barcode molecules wherein a first nucleic acid barcode molecule of the plurality of nucleic acid barcode molecules comprises a partition barcode sequence; and (d) in the partition, using the first oligonucleotide and the first nucleic acid barcode molecule to generate a first barcoded nucleic acid molecule comprising the first barcode sequence or a complement thereof and the partition barcode sequence or a complement thereof.

### Antigen-Binding Molecules

An antigen-binding molecule of the present disclosure can be any molecule capable of binding an antigen as described herein. In some embodiments, an antigen-binding molecule can be an antibody or antigen-binding fragment thereof. In some embodiments, an antigen-binding molecule can be an antibody or antigen-binding fragment thereof produced by a subject. In some embodiments, the antibody or antigen-binding fragment thereof can have affinity to an antigen provided herein. In some embodiments, the antibody or antigen-binding fragment thereof can have affinity to an antibody or antibody-based drug, for example an antibody or antibody-based drug that can be administered to a subject. In some embodiments, the antigen-binding molecule can have affinity to an antigen that is a biologic or a small molecule. For example, in some embodiments, the antigen-binding molecule can have affinity to a component of a vaccine composition.

Those skilled in the art will understand that the term “antibody” encompasses immunoglobulin (Ig), polypeptide, or protein having a binding domain which is, or is homologous to, an antigen-binding domain. The term can further include “antigen-binding fragments” and other interchangeable terms for similar binding fragments as described herein.

Native antibodies and native immunoglobulins (Igs) can be heterotetrameric glycoproteins of about 150,000 Daltons, composed of two identical light chains and two identical heavy chains. Antibodies can further refer to camelid antibodies, which can be non-tetrameric. Each light chain can be generally linked to a heavy chain by one covalent disulfide bond, while the number of disulfide linkages can vary among the heavy chains of different immunoglobulin isotypes. Each heavy and light chain can have regularly spaced intra-chain disulfide bridges. Each heavy chain can have at one end a variable domain (“VII”) followed by a number of constant domains (“C_{H}”). Each light chain can have a variable domain at one end (“V_{L}”) and a constant domain (“C_{L}”) at its other end; the constant domain of the light chain can be aligned with the first constant domain of the heavy chain, and the light-chain variable domain can be aligned with the variable domain of the heavy chain. Particular amino acid residues can form an interface between the light- and heavy-chain variable domains.

In some instances, an antibody or an antigen-binding fragment thereof includes an isolated antibody or antigen-binding fragment thereof, a purified antibody or antigen-binding fragment thereof, a recombinant antibody or antigen-binding fragment thereof, a modified antibody or antigen-binding fragment thereof, or a synthetic antibody or antigen-binding fragment thereof.

Antibodies and antigen-binding fragments herein can be partly or wholly synthetically produced. An antibody or antigen-binding fragment can be a polypeptide or protein having a binding domain which can be, or can be homologous to, an antigen-binding domain. In some instances, an antibody or an antigen-binding fragment thereof can be produced in an appropriate in vivo animal model and then isolated and/or purified.

Depending on the amino acid sequence of the constant domain of its heavy chains, immunoglobulins (Igs) can be assigned to different classes. Major classes of immunoglobulins can include: IgA, IgD, IgE, IgG, and IgM, and several of these may be further divided into subclasses (isotypes), e.g., IgG1, IgG2, IgG3, IgG4, IgA1 and IgA2. An Ig or portion thereof can, in some cases, be a human Ig. In some instances, a C_{H}3 domain can be from an immunoglobulin. In some cases, a chain or a part of an antibody or antigen-binding fragment thereof, a modified antibody or antigen-binding fragment thereof, or a binding agent can be from an Ig. In such cases, an Ig can be IgG, an IgA, an IgD, an IgE, or an IgM. In cases where the Ig is an IgG, it can be a subtype of IgG, wherein subtypes of IgG can include IgG1, an IgG2a, an IgG2b, an IgG3, and an IgG4. In some cases, a C_{H}3 domain can be from an immunoglobulin selected from the group consisting of an IgG, an IgA, an IgD, an IgE, and an IgM.

### Antigens

An antigen encompassed herein can include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. An antigen can be a molecule that can have affinity to an antigen-binding molecule. For example, an antigen can have affinity to an antibody or antigen-binding fragment thereof. In some, when contacted with an antigen-binding molecule, the antigen can bind to the antigen-binding molecule. In some embodiments, an antigen can be a biomolecule, such as a biologic therapeutic molecule. Examples of biologic therapeutic molecules can be, for example, a drug-reactive antibody or anti-drug antibody that is produced from a living organism or that contains one or more components of a living organism. A biologic therapeutic molecule can be derived from a human, animal, or microorganism using biotechnology techniques. Examples of biologic therapeutic molecules can include, for example, an immunological molecule (e.g. an antibody (such as a monoclonal antibodies), a fusion protein, a protein product of a gene therapy, a peptide, or other biologic molecule.

In some embodiments, the antigen is capable of binding to or otherwise coupling to one or more cell features or antigen-binding molecules, and can be used to characterize cells, cell features, and/or antigen-binding molecules. In some instances, cell features can include cell surface features. Cell surface features can include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof.

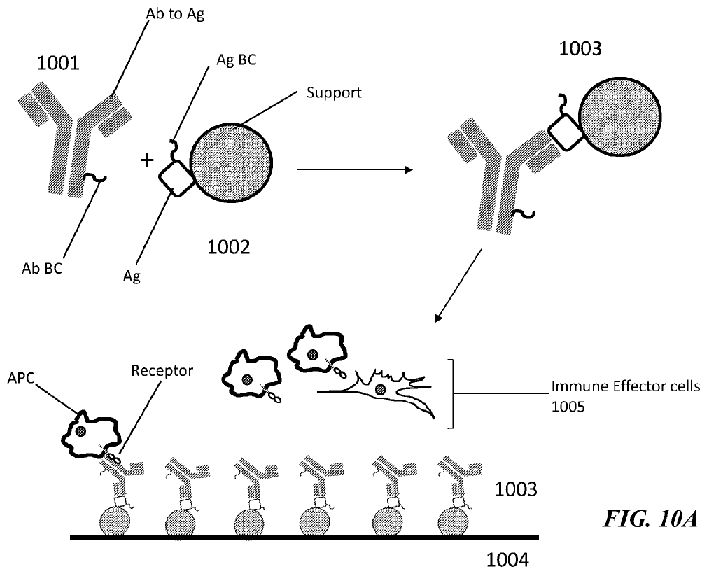

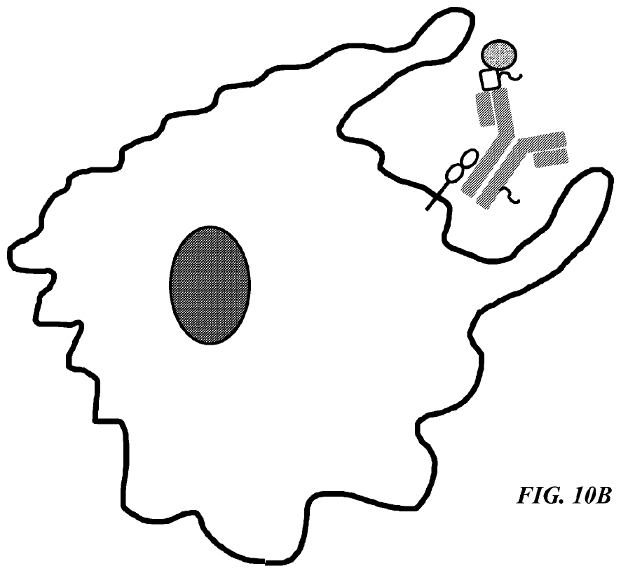

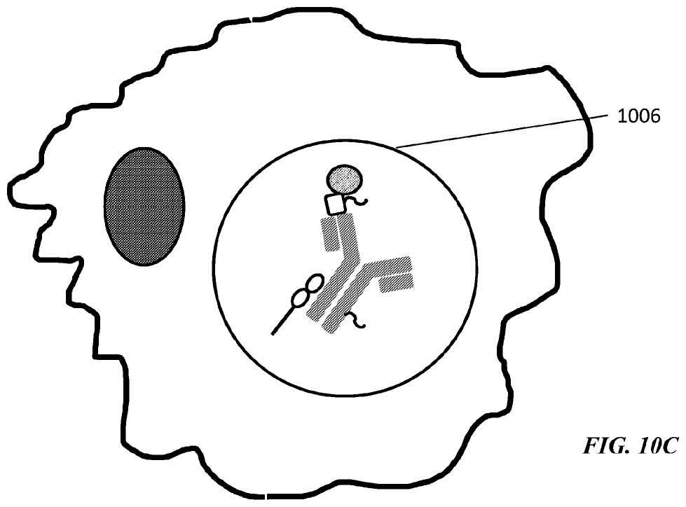

The antigen can be presented on the surface of an antigen-presenting cell (APC). Alternatively, the antigen can be conjugated to a support. An illustration of an antigen conjugated to a support is shown in FIG. 10A. In some embodiments, the support comprises a bead, as described in detail in sections below. In some exemplary embodiments, the beads are gel beads, glass beads, magnetic beads, and/or ceramic beads.

Labeling Antigen or Antigen-Binding Molecule with Barcodes

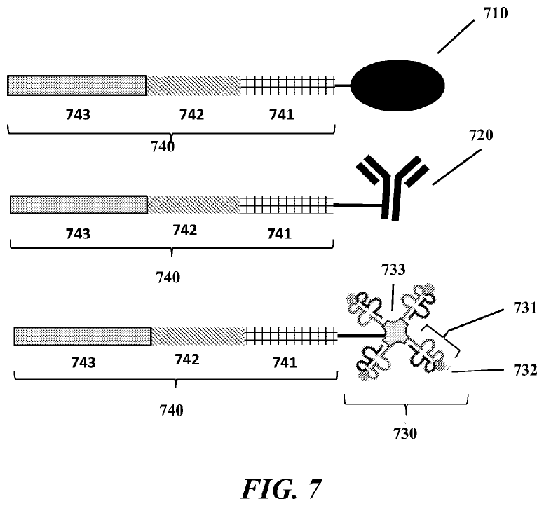

In some embodiments, the antigen or antigen-binding molecule of the present disclosure is conjugated to a barcode. For instance, FIG. 7 describes exemplary antigens or antigen-binding molecules (710, 720, or 730) conjugated to a reporter oligonucleotide (740) attached thereto. The antigen or antigen-binding molecule 710, 720, or 730 is attached (either directly, e.g., covalently attached, or indirectly) to a reporter oligonucleotide 740. A reporter oligonucleotide 740 can contain a reporter sequence 742 that identifies the antigen or antigen-binding molecule 710, 720, or 730. A reporter oligonucleotide 740 can also contain one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer biding sequence (such as an R1, R2, or partial R1 or R2 sequence).

Referring to FIG. 7, in some instances, reporter oligonucleotide 740 conjugated to an antigen (e.g., 710, 720, 730) can include a functional sequence 741 (e.g., an adaptor), a barcode sequence that identifies the antigen or antigen-binding molecule (e.g., 710, 720, 730), and functional sequence (e.g., adaptor or capture handle) 743. Capture handle 743 can be configured to hybridize to a complementary sequence (e.g., a capture sequence), such as a complementary sequence (e.g., capture sequence) present on a partition-specific barcode molecule (e.g., nucleic acid barcode molecule comprising a partition barcode sequence, not shown), such as those described elsewhere herein. A capture handle 743 can include a sequence that is complementary to a capture sequence on a partition-specific barcode molecule. In some instances, a partition-specific barcode molecule is attached to a support (e.g., a bead, such as a gel bead), such as those described elsewhere herein. For example, partition-specific barcode molecules can be attached to the support via a releasable linkage (e.g., comprising a labile bond), such as those described elsewhere herein. In some instances, a reporter oligonucleotide 740 includes one or more additional functional sequences, such as those described above. In other exemplary embodiments, the partition-specific barcode molecule can include one or more of the following: a peptide tag, an oligonucleotide barcode, a functional sequence, a common barcode, a UNIT, and a reporter capture sequence.

In some instances, antigen 710 is a protein or polypeptide (e.g., an antigen or prospective antigen) conjugated to reporter oligonucleotide 740. Reporter oligonucleotide 740 contains a reporter sequence (or reporter barcode sequence) 742 that identifies protein or polypeptide 710 and can be used to infer the presence of, e.g., a binding partner of protein or polypeptide 710 (i.e., a molecule or compound to which the protein or polypeptide binds). In some instances, 710 is a lipophilic moiety (e.g., cholesterol) comprising reporter oligonucleotide 740, where the lipophilic moiety is selected such that 710 integrates into a membrane of a cell or nucleus. Reporter oligonucleotide 740 contains reporter sequence 742 that identifies lipophilic moiety 710 which in some instances is used to tag cells (e.g., groups of cells, cell samples, etc.) for multiplex analyses as described elsewhere herein.

In some instances, the antigen-binding molecule is an antibody 720 (or an epitope binding fragment thereof) including reporter oligonucleotide 740. Reporter oligonucleotide 740 includes reporter sequence 742 that identifies antibody 720 and can be used to infer the presence of, e.g., a target of antibody 720 (i.e., a molecule or compound to which antibody 720 binds).

In some embodiments, the agent to be labeled 730 includes an MHC molecule 731 including peptide 732 and oligonucleotide 740 that identifies peptide 732. In some instances, the MHC molecule is coupled to a support 733. In some instances, support 733 is streptavidin (e.g., MHC molecule 731 can include biotin). In some embodiments, support 733 is a polysaccharide, such as dextran. In some instances, reporter oligonucleotide 740 can be directly or indirectly coupled to MEC labelling agent 730 in any suitable manner, such as to MHC molecule 731, support 733, or peptide 732. In some embodiments, labelling agent 730 includes a plurality of MHC molecules, e.g., is an MEC multimer, which can be coupled to a support (e.g., 173). There are many possible configurations of Class I and/or Class II MHC multimers that can be utilized with the compositions, methods, and systems disclosed herein, e.g., MHC tetramers, MHC pentamers (MHC assembled via a coiled-coil domain, e.g., Pro5® MHC Class I Pentamers, (ProImmune, Ltd.), MHC octamers, MHC dodecamers, MHC decorated dextran molecules (e.g., MHC Dextramer® (Immudex)), etc. For a description of exemplary labeling of various antigens, including antibody and MHC-based labelling agents, reporter oligonucleotides, and methods of use, see, e.g., U.S. Pat. No. 10,550,429 and U.S. Pat. Pub. 20190367969.

In one exemplary embodiment, the antigen-binding molecule of the present disclosure is conjugated to a reporter oligonucleotide. In certain embodiments, the reporter oligonucleotide comprises one or more of the following: a reporter capture handle, a reporter sequence, and/or a functional sequence. In some embodiments, the reporter capture handle comprises a sequence that is complementary to the reporter capture sequence, as further described herein. In another exemplary embodiment, the antigen is conjugated to a partition-specific barcode molecule. In other exemplary embodiments, the partition-specific barcode molecule can include one or more of the following: a peptide tag, an oligonucleotide barcode, a functional sequence, a common barcode, a UMI, and a reporter capture sequence.

In some embodiments, the antigen-binding molecule comprises a first oligonucleotide (e.g., first reporter oligonucleotide) comprising a first barcode sequence (e.g., first reporter barcode sequence). In some embodiments, the antigen comprises a second oligonucleotide (e.g., a second reporter oligonucleotide) comprising a second barcode sequence (e.g., second reporter barcode sequence). In some embodiments, the plurality of barcoded nucleic acid molecules further comprise the first and/or the second barcode sequence, or a complement thereof. For example, the method may comprise using a first nucleic acid barcode molecule and the first oligonucleotide to generate a first barcoded nucleic acid molecule comprising the first barcode sequence or a reverse complement thereof and the partition barcode sequence or a reverse complement thereof. The method may further comprise using a second nucleic acid barcode molecule and the second oligonucleotide to generate a second barcoded nucleic acid molecule comprising the second barcode sequence or a reverse complement thereof and the partition barcode sequence of a reverse complement thereof.

In some embodiments, a first nucleic acid barcode molecule of the plurality of nucleic acid barcode molecules includes a capture sequence configured to couple to the capture handle sequence of the reporter oligonucleotide. In some embodiments, a second nucleic acid barcode molecule of the plurality nucleic acid barcode molecules further includes a capture sequence configured to couple to an mRNA analyte. In some embodiments, the capture sequence configured to couple to the capture handle sequence of the reporter oligonucleotide is complementary to the capture handle sequence of the reporter oligonucleotide. In some embodiments, the capture sequence configured to couple to an mRNA analyte includes a polyT sequence. In some embodiments, the first and second nucleic acid barcode molecules each include a unique molecule identifier (UNIT).

In another aspect, the method of the disclosure further includes contacting the complex comprising the antigen bound to the antigen-binding molecule with a plurality of immune effector cells under conditions sufficient to provide a first immune effector cell comprising the complex as a phagocytosed complex.

### Immune Effector Cells

The term “immune effector cell” is used as its common meaning in the art, and includes all of the commonly known types of cells that are capable of modulating or effecting an immune response. Non-limiting exemplary immune effector cells include B cells, dendritic cells, natural killer cells, T cells, neutrophils, monocytes, macrophages, mast cells, monocytes, neutrophils, and/or natural killer (NK) cells, etc. In some embodiments, the plurality of immune effector cells described herein includes cells that are capable of mediating antibody-dependent cellular phagocytosis (ADCP). In some embodiments, the plurality of immune effector cells described herein includes cells that are capable of antibody-dependent cellular trogocytosis (ADCT). In some embodiments, the plurality of immune effector cells described herein includes cells that are capable of facilitating ADCP and ADCT.

In certain embodiments, the plurality of immune effector cells comprises a plurality of phagocytotic cells. In some embodiments, the plurality of immune effector cells comprises a plurality of trogocytotic cells. Those skilled in the art, upon reviewing the present disclosure, will understand the terms “phagocytotic cell” and “trogocytotic cell” as referring to a cell capable of mediating phagocytosis and trogocytosis, respectively.

In some exemplary embodiments, the plurality of phagocytic cells comprises a plurality of neutrophils, a plurality of monocytes, a plurality of macrophages, a plurality of mast cells, and/or a plurality of dendritic cells. In other exemplary embodiments, the plurality of trogocytotic cells comprises a plurality of B cells, a plurality of T cells, a plurality of monocytes, a plurality of neutrophils, and/or a plurality of natural killer (NK) cells.

In some embodiments, only a portion of the immune effector cells contain the complex of the antigen bound to the antigen-binding molecule, which is phagocytosed by the immune effector cells. In some embodiments, the complex is referred to as a phagocytosed complex. For instance, in some embodiments, a first immune effector cell contains a phagocytosed complex, while a second immune effector cell does not contain a phagocytosed complex. Thus, in some embodiments, the method further comprises separating the first immune effector cell which comprises a phagocytosed complex from the second immune effector cell which does not comprise a phagocytosed complex.

As mentioned above, in some embodiments, the antigen can be conjugated to a support. For example, see the exemplary illustration of FIG. 10A. Thus, in some embodiments, separating the first immune effector cell which comprises a phagocytosed complex from a second immune effector cell which does not comprise a phagocytosed complex is performed via the support. Referring to FIG. 10A, a barcoded antigen-binding molecule (e.g., an antibody) 1001 is contacted with a barcoded antigen conjugated to a support (e.g., a bead) 1002. The barcoded antigen-binding molecule and the barcoded antigen form an antigen-antibody complex 1003. The antigen-antibody complexes 1003 are presented on a substrate, such as a planar substrate (e.g., an array) 1004. The antigen-antibody complexes on the substrate are subsequently contacted with a plurality of immune effector cells 1005 (e.g., neutrophils, monocytes, macrophages, mast cells, or dendritic cells). Alternatively, the antigen-antibody complexes 1003 need not be presented on a substrate for contact with the plurality of immune effector cells 1005. In one embodiment, the plurality of immune effector cells 1005 may be contacted with the antigen-antibody complexes 1003 in solution. In some embodiments, at least a portion of the plurality of immune effector cells engulf (i.e., phagocytose) the antigen-antibody complex 1003, as illustrated in FIGS. 10B-10C, forming an internal compartment 1006 (e.g., a phagosome). In one embodiment, the phagocytosis occurs via binding of a surface receptor of the immune effector cell to the antibody of the antigen-antibody complex. In one embodiment, the surface receptor is an Fc receptor that binds to the Fc portion of the antibody.

The method described herein can also be used to identify trogocytotic activity or properties of an antigen-binding molecule. In certain embodiments of identifying trogocytotic antigen-binding molecules, the immune effector cells 1005 (e.g., B cells, T cells, monocytes, neutrophils, and/or NK cells) can pull the bound antigens 1003 off from the support (e.g., an array) 1004 or the APCs on which the antigens are presented.

As illustrated in FIGS. 10B-10C, in some embodiments, the cell surface receptors on the immune effector cells recognize and specifically bind to the antibody, thus mediating the phagocytosis or trogocytosis. The cells can be subsequently partitioned, lysed, and analyzed, as described in detail below.

In one aspect, trogocytotic activity or properties of an antigen-binding molecule can be determined. Referring to FIG. 10A, a barcoded antigen-binding molecule (e.g., an antibody) 1001 is contacted with a barcoded antigen conjugated to a support (e.g., a bead) 1002. The barcoded antigen-binding molecule and the barcoded antigen form an antigen-antibody complex 1003. The antigen-antibody complexes 1003 are presented on a substrate, such as a planar substrate (e.g., an array) 1004. The antigen-antibody complexes on the substrate are subsequently contacted with a plurality of immune effector cells 1005 (e.g., neutrophils, monocytes, macrophages, mast cells, or dendritic cells). Alternatively, the antigen-antibody complexes 1003 need not be presented on a substrate for contact with the plurality of immune effector cells 1005. In one embodiment, the plurality of immune effector cells 1005 may be contacted with the antigen-antibody complexes 1003 in solution. In some embodiments, at least a portion of the antigen is extracted by the antigen-binding molecule (e.g., antibody), i.e., trogocytosed, in the presence of the plurality of immune effector cells (not shown). In addition, the antigen-antigen-binding molecule complex comprising the at least a portion of the extracted antigen may be engulfed (i.e., phagocytosed) by an immune effector cell forming an internal compartment (e.g., a phagosome). In one other embodiment, the phagocytosis occurs via binding of a surface receptor of the immune effector cell to the antibody of the antigen-antibody complex. In one embodiment, the surface receptor is an Fc receptor that binds to the Fc portion of the antibody.

In some embodiments, the support comprises a cell, an exosome, or a lipoparticle.

In some embodiments, the support (e.g., the beads and/or the substrate) allows for the separating step using a density difference between the first immune effector cell that comprises a phagocytosed complex and the second immune effector cell that does not comprise a phagocytosed complex. In other embodiments, the support allows for said separating step using a magnetic difference between the first immune effector cell and the second immune effector cell. In some embodiments, the separating step is performed prior to the partitioning step. Differences in density-based or magnetic-based properties can be based on the presence of the phagocytosed cell and/or the phagocytosed support in an immune effector cell versus an immune effector cell which does not comprise phagocytosed cells/supports.

### Opsonization

In some embodiments of the method provided herein, the step of contacting the complex with a plurality of immune effector cells comprises conditions sufficient to allow opsonization of the antigen. In some embodiments, the opsonization of the antigen comprises opsonin deposition of the antigen. The term “opsonization” is used as its common meaning in the art, and refers to the process at which opsonins bind to the surface of the antigen so that the antigen will be readily identified and engulfed by phagocytes for destruction. An opsonin as encompassed herein can be any molecule that enhances phagocytosis by marking an antigen for an immune response or, in some instances, marking dead cells for recycling. For example, an opsonin as used herein can include a subset of complement components (e.g., C3b and C4b), coagulation factors, immunoglobulins (e.g., IgG, IgM, and IgE), apolipoproteins, and cell adhesion mediators, etc. An opsonin can make an antigen “visible” to immune effector cells as described herein. In one exemplary embodiment, the opsonin deposition of an antigen comprises complement deposition of the antigen. Assays to determine antibody induced complement activation or phagocytosis are known in the art, for example, discussed in Stephanie Fischingerab et al., A high-throughput, bead-based, antigen-specific assay to assess the ability of antibodies to induce complement activation. J Immunol Methods. 2019 October; 473:112630.

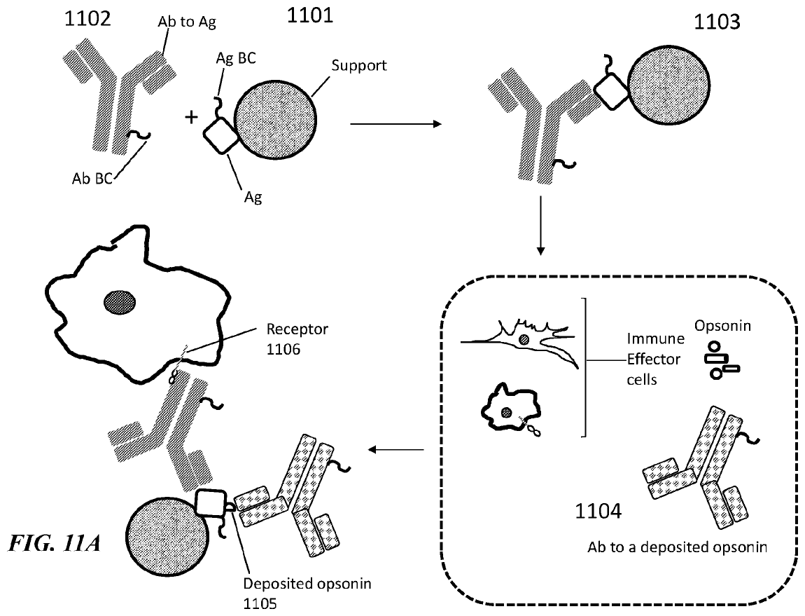

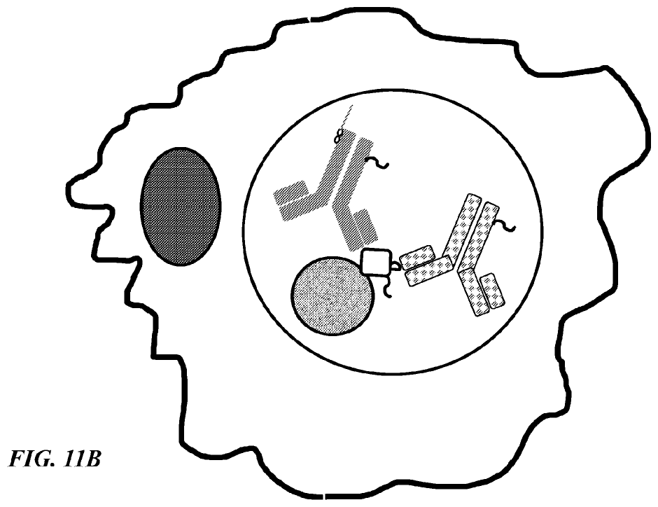

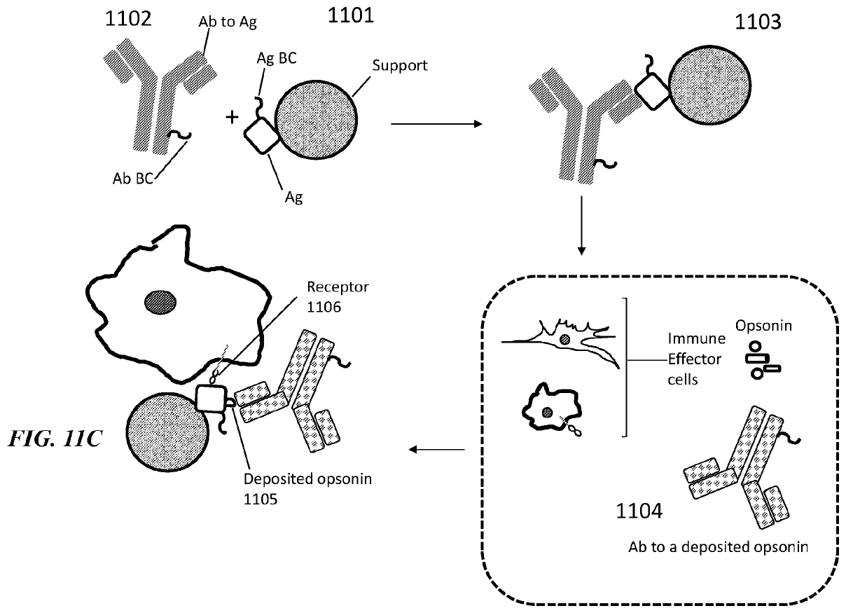

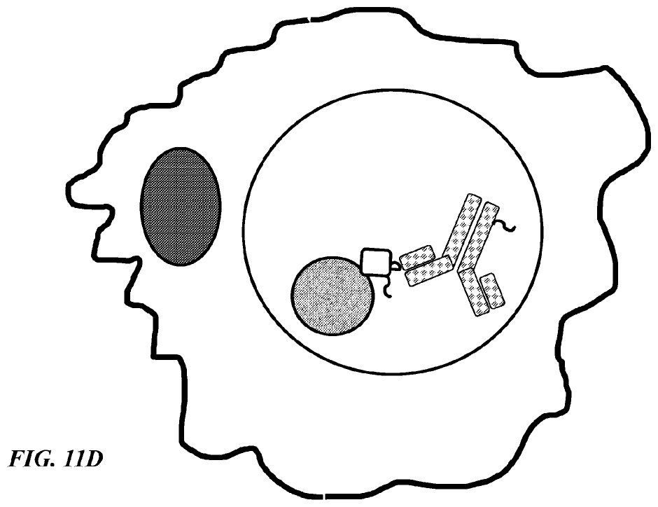

In some embodiments, the method further comprises contacting the plurality of immune effector cells with an anti-opsonin antibody. An anti-opsonin antibody as used herein can be any antibody that recognizes and specifically binds to the opsonin (e.g., a complement protein) described herein. In certain embodiments, the anti-opsonin antibody comprises an anti-complement antibody. An exemplary illustration of opsonization-mediated phagocytosis is provided in FIGS. 11A-11D. In brief, referring to FIG. 11A, a barcoded antigen 1101 is contacted with a barcoded antigen-binding molecule 1102 to form a complex comprising the antigen bound to the antigen-binding molecule 1103. Without being bound by theory, available opsonins (e.g., complement proteins such as C1, C2, and/or C4, etc.) can be deposited directly onto the antigens, as illustrated in 1105. The opsonin(s) (e.g., complement protein(s)) are deposited on or at the surface of the antigen (opsonization). In one embodiment, a barcoded antigen-binding molecule (e.g., an anti-opsonin antibody 1104) that is specific to the deposited opsonin, which is part of the complex comprising the support and the antigen-binding molecule, can be used to bind the deposited opsonin. The opsonized antigen can be recognized by the receptors (e.g., Complement Receptors, Fc Receptors) (not shown) on the surface of the phagocytic cells (e.g., CD14⁺ cells, etc.), which leads to cell activation and phagocytosis of the antigen (FIG. 11B). In another embodiment, referring to FIG. 11C, a barcoded antigen-binding molecule (e.g., an anti-opsonin antibody 1104) that is specific to the deposited opsonin, which is part of the complex comprising the support and the antigen-binding molecule, can be used to bind the deposited opsonin. The opsonized antigen can be recognized by complement receptors (FIG. 11C) on the surface of the phagocytic cells (e.g., CD14⁺ cells, etc.), which leads to cell activation and phagocytosis of the antigen and, optionally, the antigen binding molecule 1102 (FIGS. 11B and 11D). Exemplary complement receptors include, e.g., CR1 (CD35), CR2 (CD21), CR3 (e.g., a heterodimer of CD11 b and CD18), CR4 (e.g., a heterodimer of CD11c and CD18), C3AR1, and C5AR1.

In some embodiments, the anti-opsonin antibody comprises a third oligonucleotide (e.g., third reporter oligonucleotide) comprising a third barcode sequence (e.g., third reporter barcode sequence). Thus, a third barcoded nucleic acid molecule of the plurality of barcoded nucleic acid molecules can further comprise the third barcode sequence or a complement thereof. In some embodiments, the third barcoded nucleic acid molecule comprising the third barcode sequence further comprises the partition barcode sequence or reverse complement thereof. In some embodiments, the method described herein further comprises using the third oligonucleotide and the third nucleic acid barcode molecule to generate the third barcoded nucleic acid molecule comprising the third barcode sequence or a reverse complement thereof and the partition barcode sequence or a reverse complement thereof.

In some embodiments, the immune effector cell comprises a nucleic acid analyte, and a fourth nucleic acid barcode molecule of the plurality of nucleic acid barcode molecules comprises the partition barcode sequence. The method further comprises using the nucleic acid analyte and the fourth nucleic acid barcode molecule to generate a fourth barcoded nucleic acid molecule comprising a sequence of the nucleic acid analyte or a reverse complement thereof and the partition barcode sequence or a reverse complement thereof.

In some embodiments, other binding agents to complement or opsonin may be used, e.g., in lieu of an anti-opsonin antibody. These agents include, without limitation, complement family members (e.g., Factor H, C1q) that are barcoded and anti-glycan molecules.

In embodiments comprising an anti-glycan molecule, the presence of one or more glycans in a sample can be determined using a method comprising (a) incubating the sample with a glycan-specific reporter molecule comprising a glycan-specific binding moiety and a reporter oligonucleotide comprising a reporter barcode sequence, (b) partitioning the sample into a plurality of partitions such that a partition comprises (i) a single cell or single cell lysate from the sample and (ii) a plurality of nucleic acid barcode molecules comprising a partition-specific barcode sequence, and (c) using the reporter oligonucleotide and a nucleic acid barcode molecule of the plurality of nucleic acid barcode molecules to generate a first barcoded nucleic acid molecule comprising the partition-specific barcode sequence or complement thereof and the reporter barcode sequence or complement thereof.

In some embodiments, the glycan-specific binding moiety selectively binds a target glycan. In some embodiments, the reporter barcode sequence or reverse complement thereof is used to identify the target glycan. In some embodiments, the glycan-specific binding moiety selectively binds a target glycan motif. In some embodiments, the reporter barcode sequence or reverse complement thereof is used to identify the target glycan motif. In some embodiments, the glycan-specific binding moiety selectively binds a target glycan class. In some embodiments, the reporter barcode sequence or reverse complement thereof is used to identify the target glycan class.

In some embodiments, the glycan-specific binding moiety comprises an antibody that specifically binds to a target glycan, glycan motif, or glycan class, or an antigen-binding fragment thereof. In some embodiments, the antibody is a monoclonal antibody. Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

In some embodiments, the glycan-specific binding moiety is a glycan-binding protein. In some embodiments, the glycan-binding protein is selected from the group consisting of: ConA, GNA, MAL, SSA, MAH, WGA, LTL, PHA-E, GSL-II, LCA, UEA-I, AOL, AAL, LEL, DSA, ECA, PSA, TJA-I, MAL-I, SNA, PHAL, RCA120, NPA, HHL, ACG, TxLCI, BPL, TJA-II, EEL, ABA, STL, UDA, PWM, Jacalin, PNA, WFA, ACA, MPA, HPA, VVA, DBA, SBA, Calsepa, PTL-I, GSL-IA4, and GSL-IB4, or a glycan-binding fragment thereof.

In some embodiments, a glycan-specific binding moiety can be a lectin or an enzyme. For example, lectins are proteins which recognize carbohydrate domains and mainly bind to carbohydrate sugar groups. Unlike glycan binding proteins, lectins as a group do not include antibodies. Lectins bind both soluble carbohydrates and other carbohydrate moieties complexed with glycoproteins or glycolipids. As such, lectins can cause agglutination or precipitation of glycoconjugates and polysaccharides in mammals. Lectins can also mediate the attachment and binding of bacteria, viruses and fungi to their intended targets. Lectins have many functions, such as cell adhesion regulation, regulation of glycoprotein synthesis, regulation of blood protein levels, binding of glycoproteins, serve as liver cell receptors to remove certain glycoproteins from the blood stream. Further, lectins play an important part in the immune response such as their ability to mediate immune system defenses against microorganisms, their potential importance in modulations inflammatory and other immune responses. Additionally, concanavalin A, a lectin from a bean plant, has been used extensively to understand how proteins recognize carbohydrates and molecular interactions thereof. As such, their use as a glycan-specific binding moiety would be advantageous.

Another example of glycan analysis is described in Kearney et al., “SUGAR-seq Enables Simultaneous Detection of Glycans, Epitopes, and the Transcriptome in Single Cells,” Sci. Adv. 2021 7:eabe3610.

In some embodiments, the partitioning of the immune effector cells is performed according to one or more methods described in further detail below in the section entitled “Systems and Methods for Partitioning”. In some embodiments, the partitioning is performed with aid of one or more systems described in further detail below in the section entitled “Systems and Methods for Partitioning”.

In some embodiments, a partition of the plurality of partitions comprises a plurality of nucleic acid barcode molecules. The plurality of nucleic acid barcode molecules may comprise a first nucleic acid barcode molecule comprising a partition barcode sequence. The plurality of nucleic acid barcode molecules may comprise a second, third, and/or fourth nucleic acid barcode molecule comprising the partition barcode sequence.

In some embodiments, the first, second, third, and/or fourth nucleic acid barcode molecule of the plurality of nucleic acid barcode molecules comprises one or more of the following: a functional sequence, and a UMI sequence. In some embodiments, the first and second nucleic acid barcode molecules each include a unique molecule identifier (UMI).

In some embodiments, a first nucleic acid barcode molecule of the plurality of nucleic acid barcode molecules includes a capture sequence configured to couple to the capture handle sequence of the reporter oligonucleotide. In some embodiments, a second nucleic acid barcode molecule of the plurality nucleic acid barcode molecules further includes a capture sequence configured to couple to an mRNA analyte. In some embodiments, the capture sequence configured to couple to the capture handle sequence of the reporter oligonucleotide is complementary to the capture handle sequence of the reporter oligonucleotide. In some embodiments, the capture sequence configured to couple to an mRNA analyte includes a polyT sequence.

In some embodiments, the first nucleic acid barcode molecule comprises a first capture sequence configured to couple to the first oligonucleotide, and/or the second nucleic acid barcode molecule comprises a second capture sequence configured to couple to the second oligonucleotide, and/or the third nucleic acid barcode molecule comprises a third capture sequence configured to couple to the third oligonucleotide, and/or the fourth nucleic acid barcode molecule comprises a fourth capture sequence, wherein the fourth capture sequence is configured to couple to a sequence of the nucleic acid analyte or is a template switch oligonucleotide.

In some embodiments, the method comprises generating a plurality of barcoded nucleic acid molecules. Methods for generating barcoded nucleic acid molecules are described further herein.

In some embodiments, the method further comprises determining a sequence of one or more barcoded nucleic acid molecules of the plurality of barcoded nucleic acid molecules. The determining the sequence can be performed by sequencing. one or more of the barcoded nucleic acid molecules or their derivatives thereof.

In other embodiments, the downstream sequencing of the different barcoded nucleic acid molecules, or their derivatives thereof (e.g., barcoded nucleic acid molecules generated in a partition or their derivatives thereof) from a single immune effector cell can provide information about the contents of the partition and therefore properties of the antigen-binding molecules. In some embodiments, a derivative of a barcoded nucleic acid molecule is an amplicon of the barcoded nucleic acid molecule.

In one embodiment, sequencing can identify the presence of (i) the second barcode sequence or complement thereof, which indicates the presence of the antigen in the partition and (ii) the first barcode sequence or complement thereof, which indicates the presence of the antigen-binding molecule (e.g., bound to the antigen) in the partition. In an embodiment, sequencing can further identify the presence of (iii) the third barcode sequence or complement thereof, which indicates the presence of antigen-binding molecule against an opsonin (e.g., the anti-opsonin antibody 1104) in the partition. In an embodiment, the presence of the antigen-binding molecule against an opsonin in the partition indicates that the antigen has been opsonized. In another embodiment, the presence of the first and second barcode sequence or complements thereof indicates that the antigen-binding molecule has phagocytotic properties, e.g., ADCP. In some embodiments, the presence of the first, second, and third barcode sequence or complements thereof indicates that the antigen-binding molecule has opsonophagocytotic properties. In another embodiment, sequencing, e.g., of the fourth barcoded nucleic acid molecule can identify the presence of (iv) a nucleic acid analyte of the immune effector cell.

For example, in some embodiments, the determined sequence of the first barcoded nucleic acid molecule or a derivative thereof can be used to identify the antigen binding molecule as having been opsonophagocytosed and/or trogocytosed by the first immune effector cell, (ii) the determined sequence of the second barcoded nucleic acid molecule or a derivative thereof can be used to identify the antigen binding molecule as having bound the antigen, and/or (iii) the determined sequence of the third barcoded nucleic acid molecule or a derivative thereof can be used to identify the antigen as having been opsonized.

In some embodiments, the method of the disclosure includes partitioning a plurality of immune effector cells as described herein into a plurality of partitions. In some embodiments, a partition of the plurality of partitions described here comprises (i) the first immune effector cell comprising the phagocytosed complex and (ii) a plurality of nucleic acid barcode molecules. In other embodiments, a partition of the plurality of partitions described here can comprise (i) a reference immune effector cell as described in detail below and (ii) a plurality of nucleic acid barcode molecules. In some embodiments, the plurality of nucleic acid barcode molecules comprises a partition barcode sequence.

In other embodiments, the method can also include a process of sorting the plurality of immune effector cells prior to the partitioning step. In some embodiments, the sorting is conducting via a label. Any one or more of the components in the method, such as the support, the APC, the anti-complement antibody, the anti-opsonin antibody, the antigen, the antigen-binding molecule, and/or the plurality of immune effector cells can comprise the label. The label used for sorting can be a fluorophore label, a colorimetric label, a magnetic label, and/or a sortable antibody label. In some embodiments, the sortable antibody label can be conjugated to a barcode molecule. In some embodiments, the sorting results in enrichment of the immune effector cells comprising the phagocytosed complex in a sample.

In some exemplary embodiments, phagocytic cells can be sorted through microfluidics when an internalized antigen-binding molecule or antigen is detected via fluorescence, imaging, or other methods described herein. The antigen can be conjugated to a support (e.g., a bead). In another exemplary embodiments, cells can be fixed and permeabilized and another barcoded antigen-binding molecule for an opsonin (e.g., the anti-opsonin antibody 1104) can be used to detect the deposition of an opsonin on antibody or antibody-antigen internalization. See, for example, FIGS. 11A-11B.

### Systems and Methods for Partitioning

In an aspect, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions.

In some embodiments disclosed herein, the partitioned particle is a labelled cell of B-cell lineage, e.g. a plasma cell, which expresses an antigen-binding molecule (e.g., an immune receptor, an antibody or a functional fragment thereof). In other examples, the partitioned particle can be an immune effector cell, a labelled cell, or a cell engineered to express antigen-binding molecules (e.g., an immune receptors, antibodies or functional fragments thereof). In additional examples, the partitioned particle can be an immune effector cell comprising a complex of antigen bound to an antigen-binding molecule (e.g., FIG. 10C) or an immune effector cell comprising a complex of antigen bound to an antigen-binding molecule and deposited opsonin (e.g., FIG. 11B, FIG. 11D).

The term “partition,” as used herein, generally, refers to a space or volume that can be suitable to contain one or more cells, one or more species of features or compounds, or conduct one or more reactions. A partition can be a physical container, compartment, or vessel, such as a droplet, a flow cell, a reaction chamber, a reaction compartment, a tube, a well, or a microwell. In some embodiments, the compartments or partitions include partitions that are flowable within fluid streams. These partitions can include, for example, micro-vesicles that have an outer barrier surrounding an inner fluid center or core, or, in some cases, the partitions can include a porous matrix that is capable of entraining and/or retaining materials within its matrix. In some aspects, partitions comprise droplets of aqueous fluid within a non-aqueous continuous phase (e.g., oil phase). A variety of different vessels are described in, for example, U.S. Patent Application Publication No. 2014/0155295. Emulsion systems for creating stable droplets in non-aqueous or oil continuous phases are described in detail in, e.g., U.S. Patent Application Publication No. 2010/010511.

In some embodiments, a partition herein includes a space or volume that can be suitable to contain one or more species or conduct one or more reactions. A partition can be a physical compartment, such as a droplet or well. The partition can be an isolated space or volume from another space or volume. The droplet can be a first phase (e.g., aqueous phase) in a second phase (e.g., oil) immiscible with the first phase. The droplet can be a first phase in a second phase that does not phase separate from the first phase, such as, for example, a capsule or liposome in an aqueous phase. A partition can include one or more other (inner) partitions. In some cases, a partition can be a virtual compartment that can be defined and identified by an index (e.g., indexed libraries) across multiple and/or remote physical compartments. For example, a physical compartment can include a plurality of virtual compartments.

In some embodiments, the methods and system described herein provide for the compartmentalization, depositing or partitioning of individual cells from a sample material containing cells, into discrete partitions, where each partition maintains separation of its own contents from the contents of other partitions. Identifiers including unique identifiers (e.g., UMI) and common or universal tags, e.g., barcodes, can be previously, subsequently or concurrently delivered to the partitions that hold the compartmentalized or partitioned cells, in order to allow for the later attribution of the characteristics of the individual cells to one or more particular compartments. Further, identifiers including unique identifiers and common or universal tags, e.g., barcodes, can be coupled to labelling agents and previously, subsequently or concurrently delivered to the partitions that hold the compartmentalized or partitioned cells, in order to allow for the later attribution of the characteristics of the individual cells to one or more particular compartments. Identifiers including unique identifiers and common or universal tags, e.g., barcodes, can be delivered, for example on an oligonucleotide, to a partition via any suitable mechanism, for example by coupling the barcoded oligonucleotides to a support (e.g., a bead, such as a gel bead). In some embodiments, the barcoded oligonucleotides are reversibly (e.g., releasably) coupled to a support (e.g., a bead, such as a gel bead). The support suitable for the compositions and methods of the disclosure can have different surface chemistries and/or physical volumes. In some embodiments, the support includes a polymer gel. In some embodiments, the polymer gel is a polyacrylamide. Additional non-limiting examples of suitable support include microparticles, nanoparticles, cells, exosomes, lipoparticles, and beads (e.g., microbeads). In some embodiments, the support includes a bead. The partition can be a droplet in an emulsion. A partition can include one or more particles. A partition can include one or more types of particles. For example, a partition of the present disclosure can include one or more biological particles, e.g., immune effector cells, labelled engineered cells, B cells, or plasma cells, and/or macromolecular constituents thereof. A partition can include one or more gel beads. A partition can include one or more cell beads. A partition can include a single gel bead, a single cell bead, or both a single cell bead and single gel bead. A partition can include one or more reagents. Alternatively, a partition can be unoccupied. For example, a partition cannot comprise a bead. Unique identifiers, such as barcodes, can be injected into the droplets previous to, subsequent to, or concurrently with droplet generation, such as via a support (e.g., a bead, such as a gel bead), as described elsewhere herein. Microfluidic channel networks (e.g., on a chip) can be utilized to generate partitions as described herein. Alternative mechanisms can also be employed in the partitioning of individual biological particles, including porous membranes through which aqueous mixtures of cells are extruded into non-aqueous fluids.

The partitions can be flowable within fluid streams. The partitions can include, for example, micro-vesicles that have an outer barrier surrounding an inner fluid center or core. In some cases, the partitions can include a porous matrix that is capable of entraining and/or retaining materials (e.g., expressed antibodies or antigen-binding fragments thereof) within its matrix (e.g., via a capture agent configured to couple to both the matrix and the expressed antibody or antigen-binding fragment thereof). The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions can be provided in a water-in-oil emulsion or oil-in-water emulsion. A variety of different vessels are described in, for example, U.S. Patent Application Publication No. 2014/0155295. Emulsion systems for creating stable droplets in non-aqueous or oil continuous phases are described in, for example, U.S. Patent Application Publication No. 2010/0105112.

In the case of droplets in an emulsion, allocating individual particles (e.g., immune effector cells or labelled engineered cells) to discrete partitions can, in one non-limiting example, be accomplished by introducing a flowing stream of particles in an aqueous fluid into a flowing stream of a non-aqueous fluid, such that droplets are generated at the junction of the two streams. Fluid properties (e.g., fluid flow rates, fluid viscosities, etc.), particle properties (e.g., volume fraction, particle size, particle concentration, etc.), microfluidic architectures (e.g., channel geometry, etc.), and other parameters can be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions can contain less than one biological particle per partition in order to ensure that those partitions that are occupied are primarily singly occupied. In some cases, partitions among a plurality of partitions can contain at most one biological particle (e.g., bead, DNA, cell, such as an immune effector cell, a labelled engineered cells, B cells, or plasma cells, or cellular material). In some embodiments, the various parameters (e.g., fluid properties, particle properties, microfluidic architectures, etc.) can be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

In some embodiments, the method further includes individually partitioning one or more single cells (including immune effector cells or engineered cells) from a plurality of cells (including immune effector cells or engineered cells) in a partition of a second plurality of partitions.

In some embodiments, at least one of the first and second plurality of partitions includes a microwell, a flow cell, a reaction chamber, a reaction compartment, or a droplet. In some embodiments, at least one of the first and second plurality of partitions includes individual droplets in emulsion. In some embodiments, the partitions of the first plurality and/or the second plurality of partition have the same reaction volume.

In the case of droplets in emulsion, allocating individual cells to discrete partitions can generally be accomplished by introducing a flowing stream of cells in an aqueous fluid into a flowing stream of a non-aqueous fluid, such that droplets are generated at the junction of the two streams. By providing the aqueous cell-containing stream at a certain concentration of cells, the occupancy of the resulting partitions (e.g., number of cells per partition) can be controlled. For example, where single cell partitions are desired, the relative flow rates of the fluids can be selected such that, on average, the partitions contain less than one cell per partition, in order to ensure that those partitions that are occupied, are primarily singly occupied. In some embodiments, the relative flow rates of the fluids can be selected such that a majority of partitions are occupied, e.g., allowing for only a small percentage of unoccupied partitions. In some embodiments, the flows and channel architectures are controlled as to ensure a desired number of singly occupied partitions, less than a certain level of unoccupied partitions and less than a certain level of multiply occupied partitions.

In some embodiments, the methods described herein can be performed such that a majority of occupied partitions include no more than one cell per occupied partition. In some embodiments, the partitioning process is performed such that fewer than 25%, fewer than 20%, fewer than 15%, fewer than 10%, fewer than 5%, fewer than 2%, or fewer than 1% the occupied partitions contain more than one cell. In some embodiments, fewer than 20% of the occupied partitions include more than one cell. In some embodiments, fewer than 10% of the occupied partitions include more than one cell per partition. In some embodiments, fewer than 5% of the occupied partitions include more than one cell per partition. In some embodiments, it is desirable to avoid the creation of excessive numbers of empty partitions. For example, from a cost perspective and/or efficiency perspective, it may be desirable to minimize the number of empty partitions. While this can be accomplished by providing sufficient numbers of cells into the partitioning zone, the Poissonian distribution can optionally be used to increase the number of partitions that include multiple cells. As such, in some embodiments described herein, the flow of one or more of the cells, or other fluids directed into the partitioning zone are performed such that no more than 50% of the generated partitions, no more than 25% of the generated partitions, or no more than 10% of the generated partitions are unoccupied. Further, in some aspects, these flows are controlled so as to present non-Poissonian distribution of single occupied partitions while providing lower levels of unoccupied partitions. Restated, in some aspects, the above noted ranges of unoccupied partitions can be achieved while still providing any of the single occupancy rates described above. For example, in some embodiments, the use of the systems and methods described herein creates resulting partitions that have multiple occupancy rates of less than 25%, less than 20%, less than 15%), less than 10%, and in some embodiments, less than 5%, while having unoccupied partitions of less than 50%), less than 40%, less than 30%, less than 20%, less than 10%, and in some embodiments, less than 5%.

Although described in terms of providing substantially singly occupied partitions, above, in some embodiments, the methods as described herein include providing multiply occupied partitions, e.g., containing two, three, four or more cells and/or supports (e.g., beads, such as gel beads) comprising nucleic acid barcode molecules within a single partition.

In some embodiments, the reporter oligonucleotides contained within a partition are distinguishable from the reporter oligonucleotides contained within other partitions of the plurality of partitions. This can be accomplished by incorporating one or more partition-specific barcode sequences into the reporter sequence of the reporter oligonucleotides contained within the partition.

In some embodiments, it may be desirable to incorporate multiple different barcode sequences within a given partition, either attached to a single or multiple beads within the partition. For example, in some cases, a mixed, but known barcode sequences set can provide greater assurance of identification in the subsequent processing, e.g., by providing a stronger address or attribution of the barcodes to a given partition, as a duplicate or independent confirmation of the output from a given partition.

### Microfluidic Channel Structures

Microfluidic channel networks (e.g., on a chip) can be utilized to generate partitions as described herein. Alternative mechanisms can also be employed in the partitioning of individual biological particles, including porous membranes through which aqueous mixtures of cells are extruded into non-aqueous fluids.

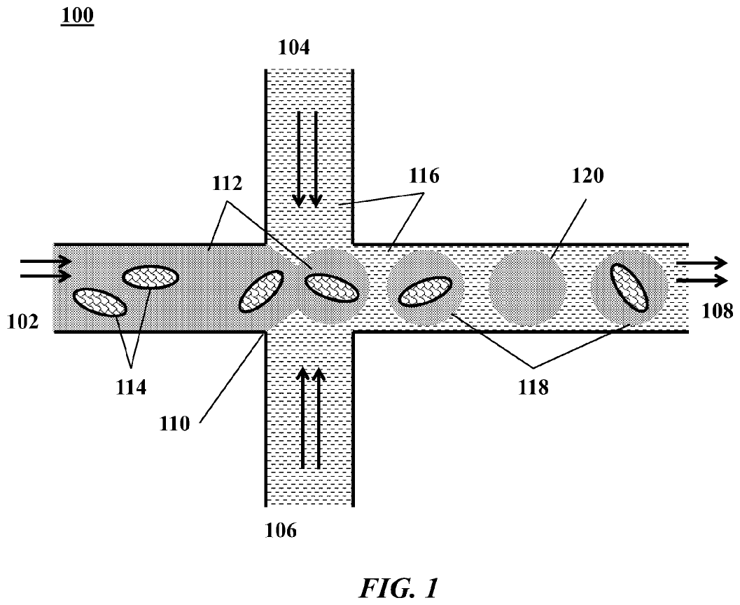

FIG. 1 shows an example of a microfluidic channel structure 100 for partitioning individual biological particles. The channel structure 100 can include channel segments 102, 104, 106 and 108 communicating at a channel junction 110. In operation, a first aqueous fluid 112 that includes suspended biological particles (e.g., cells, for example, immune effector cells, labelled engineered cells, B cells, or plasma cells) 114 can be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 can be fluidically coupled to an outlet reservoir where the discrete droplets can be stored and/or harvested. A discrete droplet generated can include an individual biological particle 114 (such as droplets 118). A discrete droplet generated can include more than one individual biological particle (e.g., immune effector cells or labelled engineered cells) 114 (not shown in FIG. 1). A discrete droplet can contain no biological particle 114 (such as droplet 120). Each discrete partition can maintain separation of its own contents (e.g., individual biological particle 114) from the contents of other partitions.

The second fluid 116 can comprise an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112.

As will be appreciated, the channel segments described herein can be coupled to any of a variety of different fluid sources or receiving components, including reservoirs, tubing, manifolds, or fluidic components of other systems. As will be appreciated, the microfluidic channel structure 100 can have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid can be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid can also or otherwise be controlled via applied pressure differentials, centrifugal force, electrokinetic pumping, vacuum, capillary or gravity flow, or the like.

The generated droplets can include two subsets of droplets: (1) occupied droplets 118, containing one or more biological particles 114, e.g., immune effector cells, labelled engineered cells, B cells, or plasma cells, and (2) unoccupied droplets 120, not containing any biological particles 114. Occupied droplets 118 can include singly occupied droplets (having one biological particle, such as one B cell or plasma cell) and multiply occupied droplets (having more than one biological particle, such as multiple B cells or plasma cells). As described elsewhere herein, in some cases, the majority of occupied partitions can include no more than one biological particle, e.g., immune effector cells, labelled engineered cells, B cells, or plasma cells, per occupied partition and some of the generated partitions can be unoccupied (of any biological particle, or labelled engineered cells, B cells, or plasma cells). In some cases, though, some of the occupied partitions can include more than one biological particle, e.g., immune effector cells, labelled engineered cells, B cells, or plasma cells. In some cases, the partitioning process can be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it can be desirable to minimize the creation of excessive numbers of empty partitions, such as to reduce costs and/or increase efficiency. While this minimization can be achieved by providing a sufficient number of biological particles (e.g., biological particles, such as immune effector cells, labelled engineered cells, B cells, or plasma cells 114) at the partitioning junction 110, such as to ensure that at least one biological particle is encapsulated in a partition, the Poissonian distribution can expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, the flow of one or more of the biological particles, such as B cells or plasma cells, (e.g., in channel segment 102), or other fluids directed into the partitioning junction (e.g., in channel segments 104, 106) can be controlled such that, in many cases, no more than about 50% of the generated partitions, no more than about 25% of the generated partitions, or no more than about 10% of the generated partitions are unoccupied. These flows can be controlled so as to present a non-Poissonian distribution of single-occupied partitions while providing lower levels of unoccupied partitions. The above noted ranges of unoccupied partitions can be achieved while still providing any of the single occupancy rates described above. For example, in many cases, the use of the systems and methods described herein can create resulting partitions that have multiple occupancy rates of less than about 25%, less than about 20%, less than about 15%, less than about 10%, and in many cases, less than about 5%, while having unoccupied partitions of less than about 50%, less than about 40%, less than about 30%, less than about 20%, less than about 10%, less than about 5%, or less.

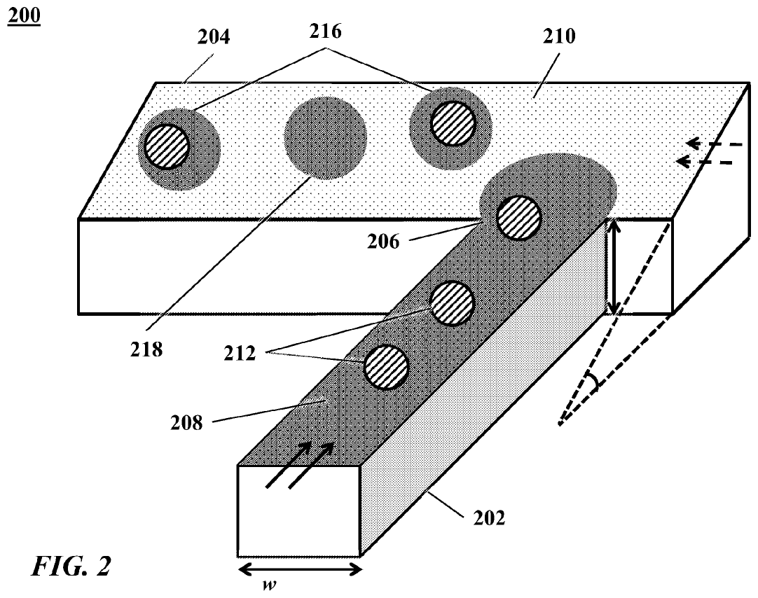

As will be appreciated, the above-described occupancy rates are also applicable to partitions that include both biological particles (e.g., immune effector cells or labelled engineered cells) and additional reagents, including, but not limited to, supports, such as beads (e.g., gel beads) carrying barcoded nucleic acid molecules (e.g., barcoded oligonucleotides) (described in relation to FIGS. 1 and 2). The occupied partitions (e.g., at least about 10%, 20%, 30%, 40%, 50%, 60%, 70%, 80%, 90%, 95%, or 99% of the occupied partitions) can include both a support (e.g., bead) comprising barcoded nucleic acid molecules and a biological particle.

In another aspect, in addition to or as an alternative to droplet based partitioning, biological particles (e.g., cells such as immune effector cells) can be encapsulated within a support (or a microcapsule) that comprises an outer shell, layer or porous matrix in which is entrained one or more individual biological particles or small groups of biological particles. The support (or microcapsule) can include other reagents. Encapsulation of biological particles, e.g., immune effector cells or labelled engineered cells, can be performed by a variety of processes. Such processes can combine an aqueous fluid containing the biological particles with a polymeric precursor material that can be capable of being formed into a gel or other solid or semi-solid matrix upon application of a particular stimulus to the polymer precursor. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), mechanical stimuli, or a combination thereof.

Preparation of supports (e.g., beads) comprising biological particles, e.g., immune effector cells, labelled engineered cells, B cells, or plasma cells, can be performed by a variety of methods. For example, air knife droplet or aerosol generators can be used to dispense droplets of precursor fluids into gelling solutions in order to form beads (e.g., gel beads) that include individual biological particles or small groups of biological particles (e.g., immune effector cells or labelled engineered cells). Likewise, membrane based encapsulation systems can be used to generate beads comprising encapsulated biological particles (e.g., immune effector cells or engineered cells) as described herein. Microfluidic systems of the present disclosure, such as that shown in FIG. 1, can be readily used in encapsulating cells as described herein. In particular, and with reference to FIG. 1, the aqueous fluid 112 comprising (i) the biological particles (e.g., immune effector cells or labelled engineered cells) 114 and (ii) the polymer precursor material (not shown) is flowed into channel junction 110, where it is partitioned into droplets 118, 120 through the flow of non-aqueous fluid 116. In the case of encapsulation methods, non-aqueous fluid 116 can also include an initiator (not shown) to cause polymerization and/or crosslinking of the polymer precursor to form the microcapsule that includes the entrained biological particles. Examples of polymer precursor/initiator pairs include those described in U.S. Patent Application Publication No. 2014/0378345.

In some cases, encapsulated biological particles can be selectively releasable from the support (or microcapsule), such as through passage of time or upon application of a particular stimulus, that degrades the support sufficiently to allow the biological particles (e.g., cells), or its other contents to be released from the support (or microcapsule), such as into a partition (e.g., droplet). See, for example, U.S. Patent Application Publication No. 2014/0378345.

### Systems and Methods for Controlled Partitioning

In some aspects, provided are systems and methods for controlled partitioning. Droplet size can be controlled by adjusting certain geometric features in channel architecture (e.g., microfluidics channel architecture). For example, an expansion angle, width, and/or length of a channel can be adjusted to control droplet size.

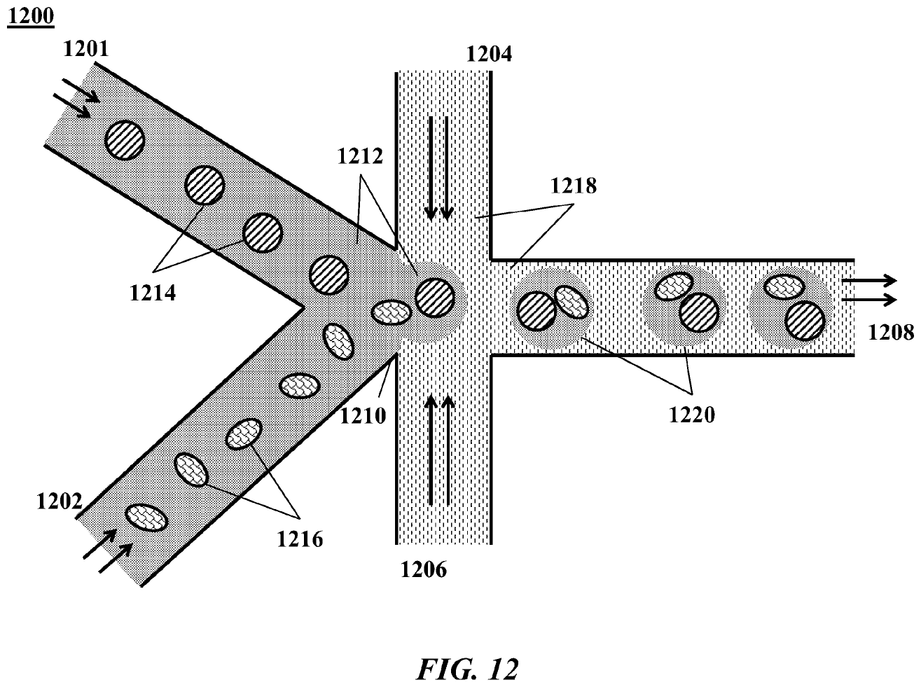

FIG. 12 shows an example of a microfluidic channel structure 1200 for delivering barcode carrying beads to droplets. The channel structure 1200 can include channel segments 1201, 1202, 1204, 1206 and 1208 communicating at a channel junction 1210. In operation, the channel segment 1201 may transport an aqueous fluid 1212 that includes a plurality of beads 1214 (e.g., with nucleic acid molecules, oligonucleotides, molecular tags) along the channel segment 1201 into junction 1210. The plurality of beads 1214 may be sourced from a suspension of beads. For example, the channel segment 1201 may be connected to a reservoir comprising an aqueous suspension of beads 1214. The channel segment 1202 may transport the aqueous fluid 1212 that includes a plurality of biological particles 12 along the channel segment 1202 into junction 1210. The plurality of biological particles 1216 may be sourced from a suspension of biological particles. For example, the channel segment 1202 may be connected to a reservoir comprising an aqueous suspension of biological particles 1216. In some instances, the aqueous fluid 1212 in either the first channel segment 1201 or the second channel segment 1202, or in both segments, can include one or more reagents, as further described below. A second fluid 1218 that is immiscible with the aqueous fluid 1212 (e.g., oil) can be delivered to the junction 1210 from each of channel segments 1204 and 1206. Upon meeting of the aqueous fluid 1212 from each of channel segments 1201 and 1202 and the second fluid 1218 from each of channel segments 1204 and 1206 at the channel junction 1210, the aqueous fluid 1212 can be partitioned as discrete droplets 1220 in the second fluid 1218 and flow away from the junction 1210 along channel segment 1208. The channel segment 1208 may deliver the discrete droplets to an outlet reservoir fluidly coupled to the channel segment 1208, where they may be harvested. As an alternative, the channel segments 1201 and 1202 may meet at another junction upstream of the junction 1210. At such junction, beads and biological particles may form a mixture that is directed along another channel to the junction 1210 to yield droplets 1220. The mixture may provide the beads and biological particles in an alternating fashion, such that, for example, a droplet comprises a single bead and a single biological particle.

FIG. 2 shows an example of a microfluidic channel structure for the controlled partitioning of beads into discrete droplets. A channel structure 200 can include a channel segment 202 communicating at a channel junction 206 (or intersection) with a reservoir 204. The reservoir 204 can be a chamber. Any reference to “reservoir,” as used herein, can also refer to a “chamber.” In operation, an aqueous fluid 208 that includes suspended beads 212 can be transported along the channel segment 202 into the junction 206 to meet a second fluid 210 that is immiscible with the aqueous fluid 208 in the reservoir 204 to create droplets 216, 218 of the aqueous fluid 208 flowing into the reservoir 204. At the junction 206 where the aqueous fluid 208 and the second fluid 210 meet, droplets can form based on factors such as the hydrodynamic forces at the junction 206, flow rates of the two fluids 208, 210, fluid properties, and certain geometric parameters (e.g., w, ho, a, etc.) of the channel structure 200. A plurality of droplets can be collected in the reservoir 204 by continuously injecting the aqueous fluid 208 from the channel segment 202 through the junction 206.

A discrete droplet generated can include a bead (e.g., as in occupied droplets 216). Alternatively, a discrete droplet generated can include more than one bead. Alternatively, a discrete droplet generated cannot include any beads (e.g., as in unoccupied droplet 218). In some instances, a discrete droplet generated can contain one or more biological particles, as described elsewhere herein. In some instances, a discrete droplet generated can include one or more reagents, as described elsewhere herein.

In some instances, the aqueous fluid 208 can have a substantially uniform concentration or frequency of beads 212. The beads 212 can be introduced into the channel segment 202 from a separate channel (not shown in FIG. 2). The frequency of beads 212 in the channel segment 202 can be controlled by controlling the frequency in which the beads 212 are introduced into the channel segment 202 and/or the relative flow rates of the fluids in the channel segment 202 and the separate channel. In some instances, the beads can be introduced into the channel segment 202 from a plurality of different channels, and the frequency controlled accordingly.

In some instances, the aqueous fluid 208 in the channel segment 202 can include biological particles (e.g., described with reference to FIG. 1). In some instances, the aqueous fluid 208 can have a substantially uniform concentration or frequency of biological particles. As with the beads, the biological particles (e.g., immune effector cells or labelled engineered cells) can be introduced into the channel segment 202 from a separate channel. The frequency or concentration of the biological particles in the aqueous fluid 208 in the channel segment 202 can be controlled by controlling the frequency in which the biological particles are introduced into the channel segment 202 and/or the relative flow rates of the fluids in the channel segment 202 and the separate channel. In some instances, the biological particles can be introduced into the channel segment 202 from a plurality of different channels, and the frequency controlled accordingly. In some instances, a first separate channel can introduce beads and a second separate channel can introduce biological particles into the channel segment 202. The first separate channel introducing the beads can be upstream or downstream of the second separate channel introducing the biological particles.

The second fluid 210 can include an oil, such as a fluorinated oil, that includes a fluorosurfactant for stabilizing the resulting droplets, for example, inhibiting subsequent coalescence of the resulting droplets.

In some instances, the second fluid 210 cannot be subjected to and/or directed to any flow in or out of the reservoir 204. For example, the second fluid 210 can be substantially stationary in the reservoir 204. In some instances, the second fluid 210 can be subjected to flow within the reservoir 204, but not in or out of the reservoir 204, such as via application of pressure to the reservoir 204 and/or as affected by the incoming flow of the aqueous fluid 208 at the junction 206. Alternatively, the second fluid 210 can be subjected and/or directed to flow in or out of the reservoir 204. For example, the reservoir 204 can be a channel directing the second fluid 210 from upstream to downstream, transporting the generated droplets.

The channel structure 200 at or near the junction 206 can have certain geometric features that at least partly determine the sizes of the droplets formed by the channel structure 200. The channel segment 202 can have a height, ho and width, w, at or near the junction 206. By way of example, the channel segment 202 can include a rectangular cross-section that leads to a reservoir 204 having a wider cross-section (such as in width or diameter). Alternatively, the cross-section of the channel segment 202 can be other shapes, such as a circular shape, trapezoidal shape, polygonal shape, or any other shapes. The top and bottom walls of the reservoir 204 at or near the junction 206 can be inclined at an expansion angle, a. The expansion angle, a, allows the tongue (portion of the aqueous fluid 208 leaving channel segment 202 at junction 206 and entering the reservoir 204 before droplet formation) to increase in depth and facilitate decrease in curvature of the intermediately formed droplet. Droplet size can decrease with increasing expansion angle. The resulting droplet radius, Rd, can be predicted by the following equation for the aforementioned geometric parameters of ho, w, and a:

$${R}_{d} \approx 0.44 ( 1 + 2.2 \sqrt{tan α} \frac{w}{{h}_{0}} ) \frac{{h}_{0}}{\sqrt{tan α}}$$

By way of example, for a channel structure with w=21 μm, h=21 μm, and α=3°, the predicted droplet size is 121 μm. In another example, for a channel structure with w=25 μm, h=μm, and α=5°, the predicted droplet size is 123 μm. In another example, for a channel structure with w=28 μm, h=28 μm, and α=7°, the predicted droplet size is 124 μm.

In some instances, the expansion angle, a, can be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, 0.01°, or less. In some instances, the width, w, can be between a range of from about 100 micrometers (nm) to about 500 nm. In some instances, the width, w, can be between a range of from about 10 μm to about 200 μm. Alternatively, the width can be less than about 10 μm. Alternatively, the width can be greater than about 500 nm. In some instances, the flow rate of the aqueous fluid 208 entering the junction 206 can be between about 0.04 microliters (μL)/minute (min) and about 40 μL/min. In some instances, the flow rate of the aqueous fluid 208 entering the junction 206 can be between about 0.01 microliters (μL)/minute (min) and about 100 μL/min. Alternatively, the flow rate of the aqueous fluid 208 entering the junction 206 can be less than about 0.01 μL/min. Alternatively, the flow rate of the aqueous fluid 208 entering the junction 206 can be greater than about 40 μL/min, such as 45 μL/min, 50 μL/min, 55 μL/min, 60 μL/min, 65 μL/min, 70 μL/min, 75 μL/min, 80 μL/min, 85 μL/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius cannot be dependent on the flow rate of the aqueous fluid 208 entering the junction 206.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

The throughput of droplet generation can be increased by increasing the points of generation, such as increasing the number of junctions (e.g., junction 206) between aqueous fluid 208 channel segments (e.g., channel segment 202) and the reservoir 204. Alternatively or in addition, the throughput of droplet generation can be increased by increasing the flow rate of the aqueous fluid 208 in the channel segment 202.

The methods and systems described herein can be used to greatly increase the efficiency of single cell applications and/or other applications receiving droplet-based input. For example, following the sorting of occupied cells and/or appropriately-sized cells, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations can occur in bulk (e.g., outside the partition). In the case where a partition is a droplet in an emulsion, the emulsion can be broken and the contents of the droplet pooled for additional operations. Additional reagents that can be co-partitioned along with the barcode bearing bead can include oligonucleotides to block ribosomal RNA (rRNA) and nucleases to digest genomic DNA from cells. Alternatively, rRNA removal agents can be applied during additional processing operations. The configuration of the constructs generated by such a method can help minimize (or avoid) sequencing of the poly-T sequence during sequencing and/or sequence the 5′ end of a polynucleotide sequence. The amplification products, for example, first amplification products and/or second amplification products, can be subject to sequencing for sequence analysis. In some cases, amplification can be performed using the Partial Hairpin Amplification for Sequencing (PHASE) method.

A variety of applications require the evaluation of the presence and quantification of different biological particle or organism types within a population of biological particles, including, for example, microbiome analysis and characterization, environmental testing, food safety testing, epidemiological analysis, e.g., in tracing contamination or the like.

Partitions including a barcode bead (e.g., a gel bead) associated with barcode molecules and a bead encapsulating cellular constituents (e.g., a cell bead) such as cellular nucleic acids can be useful in constituent analysis as is described in U.S. Patent Publication No. 2018/0216162.

### Beads

In some embodiments of the disclosure, a partition can include one or more unique identifiers, such as barcodes (e.g., a plurality of barcode nucleic acid molecules, also referred to herein as nucleic acid barcode molecules, which can be or can comprise, for example, a plurality of partition barcode sequences). Barcodes can be previously, subsequently or concurrently delivered to the partitions that hold the compartmentalized or partitioned biological particle (e.g., immune effector cells or labelled engineered cells). For example, barcodes can be injected into droplets previous to, subsequent to, or concurrently with droplet generation. In some embodiments, the delivery of the barcodes to a particular partition allows for the later attribution of the characteristics of the individual biological particle (e.g., immune effector cells or labelled engineered cells) to the particular partition. Barcodes can be delivered, for example on a nucleic acid molecule (e.g., a barcoded oligonucleotide), to a partition via any suitable mechanism. In some embodiments, barcoded nucleic acid molecules can be delivered to a partition via a support. A support, in some instances, can include a bead. Beads are described in further detail below.

In some embodiments, barcoded nucleic acid molecules can be initially associated with the support and then released from the support. In some embodiments, release of the barcoded nucleic acid molecules can be passive (e.g., by diffusion out of the support). In addition or alternatively, release from the support can be upon application of a stimulus which allows the barcoded nucleic acid nucleic acid molecules to dissociate or to be released from the support. Such stimulus can disrupt the support, an interaction that couples the barcoded nucleic acid molecules to or within the support, or both. Such stimulus can include, for example, a thermal stimulus, photo-stimulus, chemical stimulus (e.g., change in pH or use of a reducing agent), a mechanical stimulus, a radiation stimulus; a biological stimulus (e.g., enzyme), or any combination thereof. Methods and systems for partitioning barcode carrying beads into droplets are provided in US. Patent Publication Nos. 2019/0367997 and 2019/0064173, and International Application Nos. PCT/US20/17785 and PCT/US20/020486.

Beneficially, a discrete droplet partitioning a biological particle and a barcode carrying bead can effectively allow the attribution of the barcode to macromolecular constituents of the biological particle within the partition. The contents of a partition can remain discrete from the contents of other partitions.

In operation, the barcoded oligonucleotides can be released (e.g., in a partition), as described elsewhere herein. Alternatively, the nucleic acid molecules bound to the bead (e.g., gel bead) can be used to hybridize and capture analytes (e.g., one or more types of analytes) on the solid phase of the bead.

In some examples, beads, biological particles (e.g., immune effector cells or labelled engineered cells) and droplets can flow along channels (e.g., the channels of a microfluidic device), in some cases at substantially regular flow profiles (e.g., at regular flow rates). Such regular flow profiles can permit a droplet to include a single bead and a single biological particle. Such regular flow profiles can permit the droplets to have an occupancy (e.g., droplets having beads and biological particles) greater than 5%, 10%, 20%, 30%, 40%, 50%, 60%, 70%, 80%, 90%, or 95%. Such regular flow profiles and devices that can be used to provide such regular flow profiles are provided in, for example, U.S. Patent Publication No. 2015/0292988.

A bead can be porous, non-porous, solid, semi-solid, semi-fluidic, fluidic, and/or a combination thereof. In some instances, a bead can be dissolvable, disruptable, and/or degradable. In some cases, a bead cannot be degradable. In some cases, the bead can be a gel bead. A gel bead can be a hydrogel bead. A gel bead can be formed from molecular precursors, such as a polymeric or monomeric species. A semi-solid bead can be a liposomal bead. Solid beads can include metals including iron oxide, gold, and silver. In some cases, the bead can be a silica bead. In some cases, the bead can be rigid. In other cases, the bead can be flexible and/or compressible.

A bead can be of any suitable shape. Examples of bead shapes include, but are not limited to, spherical, non-spherical, oval, oblong, amorphous, circular, cylindrical, and variations thereof.

Beads can be of uniform size or heterogeneous size. In some cases, the diameter of a bead can be at least about 10 nanometers (nm), 100 nm, 500 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead can have a diameter of less than about 10 nm, 100 nm, 500 nm, 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead can have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In some embodiments, the beads described herein can have size distributions that have a coefficient of variation in their cross-sectional dimensions of less than 50%, less than 40%, less than 30%, less than 20%, and in some cases less than 15%, less than 10%, less than 5%, or less.

A bead can include natural and/or synthetic materials. For example, a bead can include a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads can also be formed from materials other than polymers, including lipids, micelles, ceramics, glass-ceramics, material composites, metals, other inorganic materials, and others.

In some embodiments, the bead can contain molecular precursors (e.g., monomers or polymers), which can form a polymer network via polymerization of the molecular precursors. In some cases, a precursor can be an already polymerized species capable of undergoing further polymerization via, for example, a chemical cross-linkage. In some embodiments, a precursor can include one or more of an acrylamide or a methacrylamide monomer, oligomer, or polymer. In some cases, the bead can include prepolymers, which are oligomers capable of further polymerization. For example, polyurethane beads can be prepared using prepolymers. In some embodiments, the bead can contain individual polymers that can be further polymerized together. In some cases, beads can be generated via polymerization of different precursors, such that they include mixed polymers, co-polymers, and/or block co-polymers. In some embodiments, the bead can include covalent or ionic bonds between polymeric precursors (e.g., monomers, oligomers, and linear polymers), nucleic acid molecules (e.g., oligonucleotides), primers, and other entities. In some embodiments, the covalent bonds can be carbon-carbon bonds, thioether bonds, or carbon-heteroatom bonds.

Cross-linking can be permanent or reversible, depending upon the particular cross-linker used. Reversible cross-linking can allow for the polymer to linearize or dissociate under appropriate conditions. In some embodiments, reversible cross-linking can also allow for reversible attachment of a material bound to the surface of a bead. In some embodiments, a cross-linker can form disulfide linkages. In some embodiments, the chemical cross-linker forming disulfide linkages can be cystamine or a modified cystamine.

In some embodiments, disulfide linkages can be formed between molecular precursor units (e.g., monomers, oligomers, or linear polymers) or precursors incorporated into a bead and nucleic acid molecules (e.g., oligonucleotides). Cystamine (including modified cystamines), for example, is an organic agent including a disulfide bond that can be used as a crosslinker agent between individual monomeric or polymeric precursors of a bead. Polyacrylamide can be polymerized in the presence of cystamine or a species including cystamine (e.g., a modified cystamine) to generate polyacrylamide gel beads including disulfide linkages (e.g., chemically degradable beads including chemically-reducible cross-linkers). The disulfide linkages can permit the bead to be degraded (or dissolved) upon exposure of the bead to a reducing agent.

In some embodiments, chitosan, a linear polysaccharide polymer, can be crosslinked with glutaraldehyde via hydrophilic chains to form a bead. Crosslinking of chitosan polymers can be achieved by chemical reactions that are initiated by heat, pressure, change in pH, and/or radiation.

In some embodiments, a bead can include an acrydite moiety, which in certain aspects can be used to attach one or more nucleic acid molecules (e.g., barcode sequence, barcoded nucleic acid molecule, barcoded oligonucleotide, primer, or other oligonucleotide) to the bead. In some cases, an acrydite moiety can refer to an acrydite analogue generated from the reaction of acrydite with one or more species, such as, the reaction of acrydite with other monomers and cross-linkers during a polymerization reaction. Acrydite moieties can be modified to form chemical bonds with a species to be attached, such as a nucleic acid molecule (e.g., barcode sequence, barcoded nucleic acid molecule, barcoded oligonucleotide, primer, or other oligonucleotide). Acrydite moieties can be modified with thiol groups capable of forming a disulfide bond or can be modified with groups already including a disulfide bond. The thiol or disulfide (via disulfide exchange) can be used as an anchor point for a species to be attached or another part of the acrydite moiety can be used for attachment. In some cases, attachment can be reversible, such that when the disulfide bond is broken (e.g., in the presence of a reducing agent), the attached species is released from the bead. In other cases, an acrydite moiety can include a reactive hydroxyl group that can be used for attachment.

Functionalization of beads for attachment of nucleic acid molecules (e.g., oligonucleotides) can be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead can include acrydite moieties, such that when a bead is generated, the bead also includes acrydite moieties. The acrydite moieties can be attached to a nucleic acid molecule (e.g., oligonucleotide), which can include a priming sequence (e.g., a primer for amplifying target nucleic acids, random primer, primer sequence for messenger RNA) and/or one or more barcode sequences. The one or more barcode sequences can include sequences that are the same for all nucleic acid molecules coupled to a given bead and/or sequences that are different across all nucleic acid molecules coupled to the given bead. The nucleic acid molecule can be incorporated into the bead.

In some embodiments, the nucleic acid molecule can include a functional sequence, for example, for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can include another functional sequence, such as, for example, a P7 sequence for attachment to a sequencing flow cell for Illumina sequencing. In some cases, the nucleic acid molecule can include a barcode sequence. In some cases, the primer can further include a unique molecular identifier (UMI). In some cases, the primer can include an R1 primer sequence for Illumina sequencing. In some cases, the primer can include an R2 primer sequence for Illumina sequencing. Examples of such nucleic acid molecules (e.g., oligonucleotides, polynucleotides, etc.) and uses thereof, as can be used with compositions, devices, methods and systems of the present disclosure, are provided in U.S. Patent Pub. Nos. 2014/0378345 and 2015/0376609.

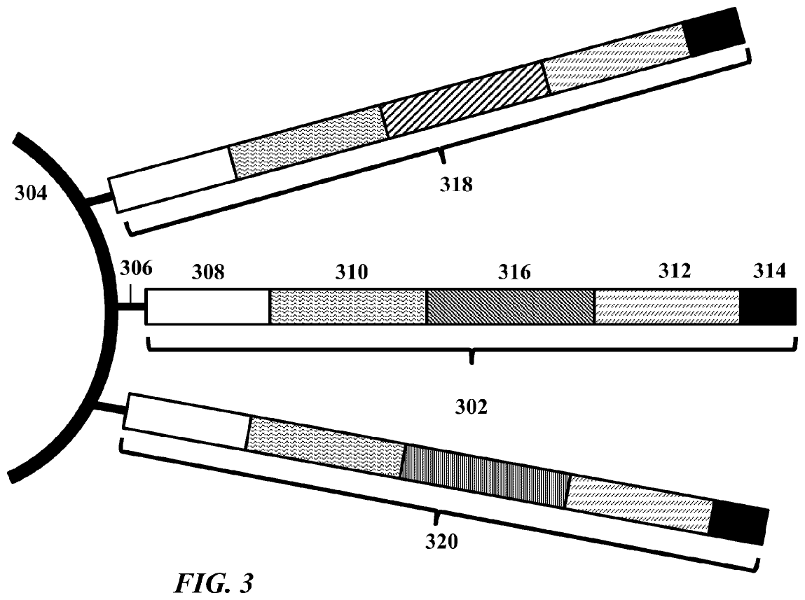

FIG. 3 illustrates an example of a barcode carrying bead. A nucleic acid molecule 302, such as an oligonucleotide, also referred to herein as a nucleic acid barcode molecule, can be coupled to a bead 304 by a releasable linkage 306, such as, for example, a disulfide linker. The same bead 304 can be coupled (e.g., via releasable linkage) to one or more other nucleic acid molecules 318, 320. The nucleic acid molecule 302 can be or include a barcode. As noted elsewhere herein, the structure of the barcode can include a number of sequence elements. The nucleic acid molecule 302 can include a functional sequence 308 that can be used in subsequent processing. For example, the functional sequence 308 can include one or more of a sequencer specific flow cell attachment sequence (e.g., a P5 sequence for Illumina® sequencing systems) and a sequencing primer sequence (e.g., a R1 primer for Illumina® sequencing systems). The nucleic acid molecule 302 can include a barcode sequence 310 for use in barcoding the sample (e.g., DNA, RNA, protein, etc.). In some cases, the barcode sequence 310 can be bead-specific such that the barcode sequence 310 is common to all nucleic acid molecules (e.g., including nucleic acid molecule 302) coupled to the same bead 304. Alternatively or in addition, the barcode sequence 310 can be partition-specific such that the barcode sequence 310 is common to all nucleic acid molecules coupled to one or more beads that are partitioned into the same partition. The nucleic acid molecule 302 can include a specific priming sequence 312, such as an mRNA specific priming sequence (e.g., poly-T sequence), a targeted priming sequence, and/or a random priming sequence. The nucleic acid molecule 302 can include an anchoring sequence 314 to ensure that the specific priming sequence 312 hybridizes at the sequence end (e.g., of the mRNA). For example, the anchoring sequence 314 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid molecule 302 can include a unique molecular identifying sequence 316 (e.g., unique molecular identifier (UMI)). In some cases, the unique molecular identifying sequence 316 can include from about 5 to about 8 nucleotides. Alternatively, the unique molecular identifying sequence 316 can compress less than about 5 or more than about 8 nucleotides. The unique molecular identifying sequence 316 can be a unique sequence that varies across individual nucleic acid molecules (e.g., 302, 318, 320, etc.) coupled to a single bead (e.g., bead 304). In some cases, the unique molecular identifying sequence 316 can be a random sequence (e.g., such as a random N-mer sequence). For example, the UMI can provide a unique identifier of the starting mRNA molecule that was captured, in order to allow quantitation of the number of original expressed RNA. As will be appreciated, although FIG. 3 shows three nucleic acid molecules 302, 318, 320 coupled to the surface of the bead 304, an individual bead can be coupled to any number of individual nucleic acid molecules, for example, from one to tens to hundreds of thousands or even millions of individual nucleic acid molecules. The respective barcodes for the individual nucleic acid molecules can include both (i) common sequence segments or relatively common sequence segments (e.g., 308, 310, 312, etc.) and (ii) variable or unique sequence segments (e.g., 316) between different individual nucleic acid molecules coupled to the same bead.

In operation, a biological particle (e.g., cell, DNA, RNA, etc.) can be co-partitioned along with a barcode bearing bead 304. The barcoded nucleic acid molecules 302, 318, 320 can be released from the bead 304 in the partition. By way of example, in the context of analyzing sample RNA, the poly-T segment (e.g., 312) of one of the released nucleic acid molecules (e.g., 302) can hybridize to the poly-A tail of a mRNA molecule. Reverse transcription can result in a cDNA transcript of the mRNA, but which transcript includes each of the sequence segments 308, 310, 316 of the nucleic acid molecule 302. Because the nucleic acid molecule 302 includes an anchoring sequence 314, it will more likely hybridize to and prime reverse transcription at the sequence end of the poly-A tail of the mRNA. Within any given partition, all of the cDNA transcripts of the individual mRNA molecules can include a common barcode sequence segment 310. However, the transcripts made from the different mRNA molecules within a given partition can vary at the unique molecular identifying sequence 312 segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UNIT segment. While a poly-T primer sequence is described, other targeted or random priming sequences can also be used in priming the reverse transcription reaction. Likewise, although described as releasing the barcoded oligonucleotides into the partition, in some cases, the nucleic acid molecules bound to the bead (e.g., gel bead) can be used to hybridize and capture the mRNA on the solid phase of the bead, for example, in order to facilitate the separation of the RNA from other cell contents. In such cases, further processing can be performed, in the partitions or outside the partitions (e.g., in bulk). For instance, the RNA molecules on the beads can be subjected to reverse transcription or other nucleic acid processing, additional adapter sequences can be added to the barcoded nucleic acid molecules, or other nucleic acid reactions (e.g., amplification, nucleic acid extension) can be performed. The beads or products thereof (e.g., barcoded nucleic acid molecules) can be collected from the partitions, and/or pooled together and subsequently subjected to clean up and further characterization (e.g., sequencing).

The operations described herein can be performed at any useful or suitable step. For instance, the beads including nucleic acid barcode molecules can be introduced into a partition (e.g., well or droplet) prior to, during, or following introduction of a sample into the partition. The nucleic acid molecules of a sample can be subjected to barcoding, which can occur on the bead (in cases where the nucleic acid molecules remain coupled to the bead) or following release of the nucleic acid barcode molecules into the partition. In cases where analytes from the sample are captured by the nucleic acid barcode molecules in a partition (e.g., by hybridization), captured analytes from various partitions may be collected, pooled, and subjected to further processing (e.g., reverse transcription, adapter attachment, amplification, clean up, sequencing). For example, in cases wherein the nucleic acid molecules from the sample remain attached to the bead, the beads from various partitions can be collected, pooled, and subjected to further processing (e.g., reverse transcription, adapter attachment, amplification, clean up, and/or sequencing). In other instances, the processing can occur in the partition. For example, conditions sufficient for barcoding, adapter attachment, reverse transcription, or other nucleic acid processing operations can be provided in the partition and performed prior to clean up and sequencing.

In some instances, a bead can include a capture sequence or binding sequence configured to bind to a corresponding capture sequence or binding sequence. In some instances, a bead can include a plurality of different capture sequences or binding sequences configured to bind to different respective corresponding capture sequences or binding sequences. For example, a bead can include a first subset of one or more capture sequences each configured to bind to a first corresponding capture sequence, a second subset of one or more capture sequences each configured to bind to a second corresponding capture sequence, a third subset of one or more capture sequences each configured to bind to a third corresponding capture sequence, and etc. A bead can include any number of different capture sequences. In some instances, a bead can include at least 2, 3, 4, 5, 6, 7, 8, 9, 10 or more different capture sequences or binding sequences configured to bind to different respective capture sequences or binding sequences, respectively. Alternatively or in addition, a bead can include at most about 10, 9, 8, 7, 6, 5, 4, 3, or 2 different capture sequences or binding sequences configured to bind to different respective capture sequences or binding sequences. In some instances, the different capture sequences or binding sequences can be configured to facilitate analysis of a same type of analyte. In some instances, the different capture sequences or binding sequences can be configured to facilitate analysis of different types of analytes (with the same bead). The capture sequence can be designed to attach to a corresponding capture sequence. Beneficially, such corresponding capture sequence can be introduced to, or otherwise induced in, a biological particle (e.g., cell, cell bead, etc.) for performing different assays in various formats (e.g., barcoded antibodies including the corresponding capture sequence, barcoded MHC dextramers including the corresponding capture sequence, barcoded guide RNA molecules including the corresponding capture sequence, etc.), such that the corresponding capture sequence can later interact with the capture sequence associated with the bead. In some instances, a capture sequence coupled to a bead (or other support) can be configured to attach to a linker molecule, such as a splint molecule, wherein the linker molecule is configured to couple the bead (or other support) to other molecules through the linker molecule, such as to one or more analytes or one or more other linker molecules.

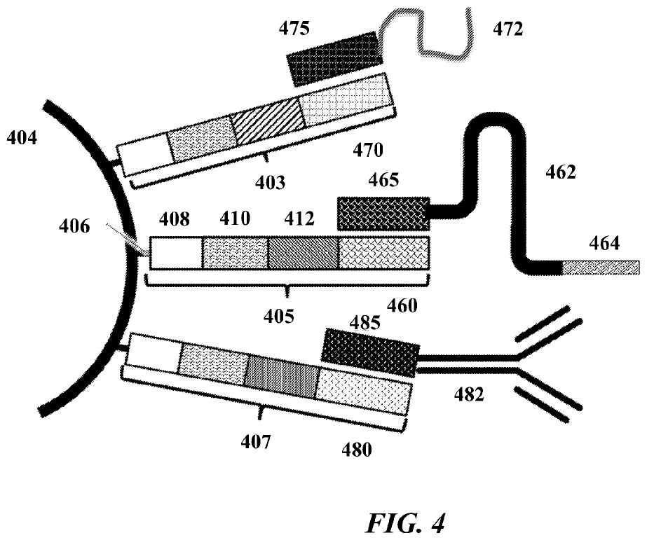

FIG. 4 illustrates a non-limiting example of a barcode carrying bead in accordance with some embodiments of the disclosure. A nucleic acid molecule 405, such as an oligonucleotide (also referred to herein as a nucleic acid barcode molecule), can be coupled to a bead 404 by a releasable linkage 406, such as, for example, a disulfide linker. The nucleic acid molecule 405 can include a first capture sequence 460. The same bead 404 can be coupled, e.g., via releasable linkage, to one or more other nucleic acid molecules 403, 407 including other capture sequences. The nucleic acid molecule 405 can be or include a barcode. As described elsewhere herein, the structure of the barcode can include a number of sequence elements, such as a functional sequence 408 (e.g., flow cell attachment sequence, sequencing primer sequence, etc.), a barcode sequence 410 (e.g., bead-specific sequence common to bead, partition-specific sequence common to partition, etc.), and a unique molecular identifier 412 (e.g., unique sequence within different molecules attached to the bead), or partial sequences thereof. The capture sequence 460 can be configured to attach to a corresponding capture sequence 465 (e.g., capture handle). In some instances, the corresponding capture sequence 465 can be coupled to another molecule that can be an analyte or an intermediary carrier. For example, as illustrated in FIG. 4, the corresponding capture sequence 465 is coupled to a guide RNA molecule 462 including a target sequence 464, wherein the target sequence 464 is configured to attach to the analyte. Another oligonucleotide molecule 407 attached to the bead 404 includes a second capture sequence 480 which is configured to attach to a second corresponding capture sequence (e.g., capture handle) 485. As illustrated in FIG. 4, the second corresponding capture sequence 485 is coupled to an antibody 482. In some cases, the antibody 482 can have binding specificity to an analyte (e.g., surface protein). Alternatively, the antibody 482 cannot have binding specificity. Another oligonucleotide molecule 403 attached to the bead 404 includes a third capture sequence 470 which is configured to attach to a second corresponding capture sequence 475. As illustrated in FIG. 4, the third corresponding capture sequence (e.g., capture handle) 475 is coupled to a molecule 472. The molecule 472 may or may not be configured to target an analyte. The other oligonucleotide molecules 403, 407 can include the other sequences (e.g., functional sequence, barcode sequence, UMI, etc.) described with respect to oligonucleotide molecule 405. While a single oligonucleotide molecule including each capture sequence is illustrated in FIG. 4, it will be appreciated that, for each capture sequence, the bead can include a set of one or more oligonucleotide molecules each including the capture sequence. For example, the bead can include any number of sets of one or more different capture sequences. Alternatively or in addition, the bead 404 can include other capture sequences. Alternatively or in addition, the bead 404 can include fewer types of capture sequences (e.g., two capture sequences). Alternatively or in addition, the bead 404 can include oligonucleotide molecule(s) including a priming sequence, such as a specific priming sequence such as an mRNA specific priming sequence (e.g., poly-T sequence), a targeted priming sequence, and/or a random priming sequence, for example, to facilitate an assay for gene expression.

The generation of a barcoded sequence, see, e.g., FIG. 3, is described herein.

In some embodiments, precursors including a functional group that is reactive or capable of being activated such that it becomes reactive can be polymerized with other precursors to generate gel beads including the activated or activatable functional group. The functional group can then be used to attach additional species (e.g., disulfide linkers, primers, other oligonucleotides, etc.) to the gel beads. For example, some precursors including a carboxylic acid (COOH) group can co-polymerize with other precursors to form a gel bead that also includes a COOH functional group. In some cases, acrylic acid (a species including free COOH groups), acrylamide, and bis(acryloyl)cystamine can be co-polymerized together to generate a gel bead including free COOH groups. The COOH groups of the gel bead can be activated (e.g., via 1-Ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species including an amine functional group where the carboxylic acid groups are activated to be reactive with an amine functional group) including a moiety to be linked to the bead.

Beads including disulfide linkages in their polymeric network can be functionalized with additional species via reduction of some of the disulfide linkages to free thiols (see e.g., U.S. patent Ser. No. 10/323,279).

A bead injected or otherwise introduced into a partition can include releasably, cleavably, or reversibly attached barcodes (e.g., partition barcode sequences). A bead injected or otherwise introduced into a partition can include activatable barcodes. A bead injected or otherwise introduced into a partition can be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage can be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes can sometimes be referred to as being activatable, in that they are available for reaction once released. Thus, for example, an activatable barcode can be activated by releasing the barcode from a bead (or other suitable type of partition described herein). Other activatable configurations are also envisioned in the context of the described methods and systems.

In addition to, or as an alternative to the cleavable linkages between the beads and the associated molecules, such as barcode containing nucleic acid molecules (e.g., barcoded oligonucleotides), the beads can be degradable, disruptable, or dissolvable spontaneously or upon exposure to one or more stimuli (e.g., temperature changes, pH changes, exposure to particular chemical species or phase, exposure to light, reducing agent, etc.). In some cases, a bead can be dissolvable, such that material components of the beads are solubilized when exposed to a particular chemical species or an environmental change, such as a change temperature or a change in pH. In some cases, a gel bead can be degraded or dissolved at elevated temperature and/or in basic conditions. In some cases, a bead can be thermally degradable such that when the bead is exposed to an appropriate change in temperature (e.g., heat), the bead degrades. Degradation or dissolution of a bead bound to a species (e.g., a nucleic acid molecule, e.g., barcoded oligonucleotide) can result in release of the species from the bead.

As will be appreciated from the above disclosure, the degradation of a bead can refer to the disassociation of a bound (e.g., capture agent configured to couple to a secreted antibody or antigen-binding fragment thereof) or entrained species (e.g., immune effector cells or labelled engineered cells, B cells, or plasma cells, or secreted antibody or antigen-binding fragment thereof) from a bead, both with and without structurally degrading the physical bead itself. For example, the degradation of the bead can involve cleavage of a cleavable linkage via one or more species and/or methods described elsewhere herein. In another example, entrained species can be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead can cause a bead to better retain an entrained species due to pore size contraction.

A degradable bead can be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) can interact with other reagents contained in the partition. For example, a polyacrylamide bead including cystamine and linked, via a disulfide bond, to a barcode sequence, can be combined with a reducing agent within a droplet of a water-in-oil emulsion. Within the droplet, the reducing agent can break the various disulfide bonds, resulting in bead degradation and release of the barcode sequence into the aqueous, inner environment of the droplet. In another example, heating of a droplet including a bead-bound barcode sequence in basic solution can also result in bead degradation and release of the attached barcode sequence into the aqueous, inner environment of the droplet.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration can be selected to facilitate certain reactions for generating a sequencing library, e.g., amplification, within the partition. In some cases, the pre-defined concentration of the primer can be limited by the process of producing nucleic acid molecule (e.g., oligonucleotide) bearing beads.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads can be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads can be accomplished by various swelling methods. The de-swelling of the beads can be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads can be accomplished by various de-swelling methods. Transferring the beads can cause pores in the bead to shrink. The shrinking can then hinder reagents within the beads from diffusing out of the interiors of the beads. The hindrance can be due to steric interactions between the reagents and the interiors of the beads. The transfer can be accomplished microfluidically. For instance, the transfer can be achieved by moving the beads from one co-flowing solvent stream to a different co-flowing solvent stream. The swellability and/or pore size of the beads can be adjusted by changing the polymer composition of the bead.

In some cases, an acrydite moiety linked to a precursor, another species linked to a precursor, or a precursor itself can include a labile bond, such as chemically, thermally, or photo-sensitive bond e.g., disulfide bond, UV sensitive bond, or the like. Once acrydite moieties or other moieties including a labile bond are incorporated into a bead, the bead can also include the labile bond. The labile bond can be, for example, useful in reversibly linking (e.g., covalently linking) species (e.g., barcodes, primers, etc.) to a bead. In some cases, a thermally labile bond can include a nucleic acid hybridization based attachment, e.g., where an oligonucleotide is hybridized to a complementary sequence that is attached to the bead, such that thermal melting of the hybrid releases the oligonucleotide, e.g., a barcode containing sequence, from the support (e.g., a bead such as a gel bead).

The addition of multiple types of labile bonds to a gel bead can result in the generation of a bead capable of responding to varied stimuli. Each type of labile bond can be sensitive to an associated stimulus (e.g., chemical stimulus, light, temperature, enzymatic, etc.) such that release of species attached to a bead via each labile bond can be controlled by the application of the appropriate stimulus. Such functionality can be useful in controlled release of species from a gel bead. In some cases, another species including a labile bond can be linked to a gel bead after gel bead formation via, for example, an activated functional group of the gel bead as described above. As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

The barcodes that are releasable as described herein can sometimes be referred to as being activatable, in that they are available for reaction once released. Thus, for example, an activatable barcode can be activated by releasing the barcode from a bead (or other suitable type of partition described herein). Other activatable configurations are also envisioned in the context of the described methods and systems.

In addition to thermally cleavable bonds, disulfide bonds and UV sensitive bonds, other non-limiting examples of labile bonds that can be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)). A bond can be cleavable via other nucleic acid molecule targeting enzymes, such as restriction enzymes (e.g., restriction endonucleases), as described further below.

Species can be encapsulated in beads (e.g., capture agent) during bead generation (e.g., during polymerization of precursors). Such species may or may not participate in polymerization. Such species can be entered into polymerization reaction mixtures such that generated beads include the species upon bead formation. In some cases, such species can be added to the gel beads after formation. Such species can include, for example, nucleic acid molecules (e.g., oligonucleotides), reagents for a nucleic acid amplification reaction (e.g., primers, polymerases, dNTPs, co-factors (e.g., ionic co-factors, buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species can include one or more enzymes described herein, including without limitation, polymerase, reverse transcriptase, restriction enzymes (e.g., endonuclease), transposase, ligase, proteinase K, DNAse, etc. Such species can include one or more reagents described elsewhere herein (e.g., lysis agents, inhibitors, inactivating agents, chelating agents, stimulus). Trapping of such species can be controlled by the polymer network density generated during polymerization of precursors, control of ionic charge within the gel bead (e.g., via ionic species linked to polymerized species), or by the release of other species. Encapsulated species can be released from a bead upon bead degradation and/or by application of a stimulus capable of releasing the species from the bead. Alternatively or in addition, species can be partitioned in a partition (e.g., droplet) during or subsequent to partition formation. Such species can include, without limitation, the abovementioned species that can also be encapsulated in a bead.

A degradable bead can include one or more species with a labile bond such that, when the bead/species is exposed to the appropriate stimuli, the bond is broken and the bead degrades. The labile bond can be a chemical bond (e.g., covalent bond, ionic bond) or can be another type of physical interaction (e.g., van der Waals interactions, dipole-dipole interactions, etc.). In some cases, a crosslinker used to generate a bead can include a labile bond. Upon exposure to the appropriate conditions, the labile bond can be broken and the bead degraded. For example, upon exposure of a polyacrylamide gel bead including cystamine crosslinkers to a reducing agent, the disulfide bonds of the cystamine can be broken and the bead degraded.

A degradable bead can be useful in more quickly releasing an attached species (e.g., a nucleic acid molecule, a barcode sequence, a primer, etc.) from the bead when the appropriate stimulus is applied to the bead as compared to a bead that does not degrade. For example, for a species bound to an inner surface of a porous bead or in the case of an encapsulated species, the species can have greater mobility and accessibility to other species in solution upon degradation of the bead. In some cases, a species can also be attached to a degradable bead via a degradable linker (e.g., disulfide linker). The degradable linker can respond to the same stimuli as the degradable bead or the two degradable species can respond to different stimuli. For example, a barcode sequence can be attached, via a disulfide bond, to a polyacrylamide bead including cystamine. Upon exposure of the barcoded-bead to a reducing agent, the bead degrades and the barcode sequence is released upon breakage of both the disulfide linkage between the barcode sequence and the bead and the disulfide linkages of the cystamine in the bead.

As will be appreciated from the above disclosure, while referred to as degradation of a bead, in many instances as noted above, that degradation can refer to the disassociation of a bound or entrained species from a bead, both with and without structurally degrading the physical bead itself. For example, entrained species can be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead can cause a bead to better retain an entrained species due to pore size contraction.

Where degradable beads are provided, it can be beneficial to avoid exposing such beads to the stimulus or stimuli that cause such degradation prior to a given time, in order to, for example, avoid premature bead degradation and issues that arise from such degradation, including for example poor flow characteristics and aggregation. By way of example, where beads include reducible cross-linking groups, such as disulfide groups, it will be desirable to avoid contacting such beads with reducing agents, e.g., DTT or other disulfide cleaving reagents. In such cases, treatment to the beads described herein will, in some cases be provided free of reducing agents, such as DTT. Because reducing agents are often provided in commercial enzyme preparations, it can be desirable to provide reducing agent free (or DTT free) enzyme preparations in treating the beads described herein. Examples of such enzymes include, e.g., polymerase enzyme preparations, reverse transcriptase enzyme preparations, ligase enzyme preparations, as well as many other enzyme preparations that can be used to treat the beads described herein. The terms “reducing agent free” or “DTT free” preparations can refer to a preparation having less than about 1/10th, less than about 1/50th, or even less than about 1/100th of the lower ranges for such materials used in degrading the beads. For example, for DTT, the reducing agent free preparation can have less than about 0.01 millimolar (mM), 0.005 mM, 0.001 mM DTT, 0.0005 mM DTT, or even less than about 0.0001 mM DTT. In many cases, the amount of DTT can be undetectable.

Numerous chemical triggers can be used to trigger the degradation of beads. Examples of these chemical changes can include, but are not limited to pH-mediated changes to the integrity of a component within the bead, degradation of a component of a bead via cleavage of cross-linked bonds, and depolymerization of a component of a bead.

In some embodiments, a bead can be formed from materials that include degradable chemical crosslinkers, such as BAC or cystamine. Degradation of such degradable crosslinkers can be accomplished through a number of mechanisms (see e.g., U.S. patent Ser. No. 10/323,279).

Beads can also be induced to release their contents upon the application of a thermal stimulus. A change in temperature can cause a variety of changes to a bead. For example, heat can cause a solid bead to liquefy. A change in heat can cause melting of a bead such that a portion of the bead degrades. In other cases, heat can increase the internal pressure of the bead components such that the bead ruptures or explodes. Heat can also act upon heat-sensitive polymers used as materials to construct beads.

Any suitable agent can degrade beads. In some embodiments, changes in temperature or pH can be used to degrade thermo-sensitive or pH-sensitive bonds within beads. In some embodiments, chemical degrading agents can be used to degrade chemical bonds within beads by oxidation, reduction or other chemical changes. For example, a chemical degrading agent can be a reducing agent, such as DTT, wherein DTT can degrade the disulfide bonds formed between a crosslinker and gel precursors, thus degrading the bead. In some embodiments, a reducing agent can be added to degrade the bead, which may or may not cause the bead to release its contents. Examples of reducing agents can include dithiothreitol (DTT), (3-mercaptoethanol, (2S)-2-amino-1,4-dimercaptobutane (dithiobutylamine or DTBA), tris(2-carboxyethyl) phosphine (TCEP), or combinations thereof. The reducing agent can be present at a concentration of about 0.1 mM, 0.5 mM, 1 mM, 5 mM, or 10 mM. The reducing agent can be present at a concentration of at least about 0.1 mM, 0.5 mM, 1 mM, 5 mM, 10 mM, or greater than 10 mM. The reducing agent can be present at concentration of at most about 10 mM, 5 mM, 1 mM, 0.5 mM, 0.1 mM, or less.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration can be selected to facilitate certain reactions for generating a sequencing library, e.g., amplification, within the partition. In some cases, the pre-defined concentration of the primer can be limited by the process of producing oligonucleotide bearing beads.

Although FIG. 1, FIG. 2, and FIG. 12 have been described in terms of providing substantially singly occupied partitions, above, in certain cases, it may be desirable to provide multiply occupied partitions, e.g., containing two, three, four or more cells and/or supports (e.g., beads) including barcoded nucleic acid molecules (e.g., oligonucleotides) within a single partition (e.g., multi-omics method described elsewhere, herein). Accordingly, as noted above, the flow characteristics of the biological particle and/or bead containing fluids and partitioning fluids can be controlled to provide for such multiply occupied partitions. In particular, the flow parameters can be controlled to provide a given occupancy rate at greater than about 50% of the partitions, greater than about 75%, and in some cases greater than about 80%, 90%, 95%, or higher.

In some cases, additional supports can be used to deliver additional reagents to a partition. In such cases, it can be advantageous to introduce different beads into a common channel or droplet generation junction, from different bead sources (e.g., containing different associated reagents) through different channel inlets into such common channel or droplet generation junction (e.g., junction 210). In such cases, the flow and frequency of the different beads into the channel or junction can be controlled to provide for a certain ratio of supports from each source, while ensuring a given pairing or combination of such beads into a partition with a given number of biological particles (e.g., one biological particle and one bead per partition).

The partitions described herein can include small volumes, for example, less than about microliters (μL), 5 μL, 1 μL, 900 picoliters (μL), 800 μL, 700 μL, 600 μL, 500 μL, 400 μL, 300 μL, 200 μL, 100 μL, 50 μL, 20 μL, 10 μL, 1 μL, 500 nanoliters (nL), 100 nL, 50 nL, or less.

For example, in the case of droplet based partitions, the droplets can have overall volumes that are less than about 1000 μL, 900 μL, 800 μL, 700 μL, 600 μL, 500 μL, 400 μL, 300 μL, 200 μL, 100 μL, 50 μL, 20 μL, 10 μL, 1 μL, or less. Where co-partitioned with supports, it will be appreciated that the sample fluid volume, e.g., including co-partitioned biological particles and/or beads, within the partitions can be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species can generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions can include both unoccupied partitions (e.g., empty partitions) and occupied partitions.

### Microwells

As described herein, one or more processes can be performed in a partition, which can be a well. The well can be a well of a plurality of wells of a substrate, such as a microwell of a microwell array or plate, or the well can be a microwell or microchamber of a device (e.g., microfluidic device) comprising a substrate. The well can be a well of a well array or plate, or the well can be a well or chamber of a device (e.g., fluidic device). Accordingly, the wells or microwells can assume an “open” configuration, in which the wells or microwells are exposed to the environment (e.g., contain an open surface) and are accessible on one planar face of the substrate, or the wells or microwells can assume a “closed” or “sealed” configuration, in which the microwells are not accessible on a planar face of the substrate. In some instances, the wells or microwells can be configured to toggle between “open” and “closed” configurations. For instance, an “open” microwell or set of microwells can be “closed” or “sealed” using a membrane (e.g., semi-permeable membrane), an oil (e.g., fluorinated oil to cover an aqueous solution), or a lid, as described elsewhere herein. The wells or microwells can be initially provided in a “closed” or “sealed” configuration, wherein they are not accessible on a planar surface of the substrate without an external force. For instance, the “closed” or “sealed” configuration can include a substrate such as a sealing film or foil that is puncturable or pierceable by pipette tip(s). Suitable materials for the substrate include, without limitation, polyester, polypropylene, polyethylene, vinyl, and aluminum foil.

In some embodiments, the well can have a volume of less than 1 milliliter (mL). For example, the well can be configured to hold a volume of at most 1000 microliters (μL), at most 100 μL, at most 10 μL, at most 1 μL, at most 100 nanoliters (nL), at most 10 nL, at most 1 nL, at most 100 picoliters (pL), at most 10 (pL), or less. The well can be configured to hold a volume of about 1000 μL, about 100 μL, about 10 μL, about 1 μL, about 100 nL, about 10 nL, about 1 nL, about 100 pL, about 10 pL, etc. The well can be configured to hold a volume of at least 10 pL, at least 100 pL, at least 1 nL, at least 10 nL, at least 100 nL, at least 1 μL, at least 10 μL, at least 100 μL, at least 1000 μL, or more. The well can be configured to hold a volume in a range of volumes listed herein, for example, from about 5 nL to about 20 nL, from about 1 nL to about 100 nL, from about 500 pL to about 100 μL, etc. The well can be of a plurality of wells that have varying volumes and can be configured to hold a volume appropriate to accommodate any of the partition volumes described herein.

In some instances, a microwell array or plate includes a single variety of microwells. In some instances, a microwell array or plate includes a variety of microwells. For instance, the microwell array or plate can include one or more types of microwells within a single microwell array or plate. The types of microwells can have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate can include any number of different types of microwells. For example, the microwell array or plate can include 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well can have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate includes different types of microwells that are located adjacent to one another within the array or plate. For example, a microwell with one set of dimensions can be located adjacent to and in contact with another microwell with a different set of dimensions. Similarly, microwells of different geometries can be placed adjacent to or in contact with one another. The adjacent microwells can be configured to hold different articles; for example, one microwell can be used to contain a cell, cell bead, or other sample (e.g., cellular components, nucleic acid molecules, etc.) while the adjacent microwell can be used to contain a support (e.g., a bead such as a gel bead), droplet, or other reagent. In some cases, the adjacent microwells can be configured to merge the contents held within, e.g., upon application of a stimulus, or spontaneously, upon contact of the articles in each microwell.

As is described elsewhere herein, a plurality of partitions can be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells can include both unoccupied wells (e.g., empty wells) and occupied wells.

A well can include any of the reagents described herein, or combinations thereof. These reagents can include, for example, barcode molecules, enzymes, adapters, and combinations thereof. The reagents can be physically separated from a sample (for example, a cell, cell bead, or cellular components, e.g., proteins, nucleic acid molecules, etc.) that is placed in the well. This physical separation can be accomplished by containing the reagents within, or coupling to, a support (e.g., a bead such as a gel bead) that is placed within a well. The physical separation can also be accomplished by dispensing the reagents in the well and overlaying the reagents with a layer that is, for example, dissolvable, meltable, or permeable prior to introducing the polynucleotide sample into the well. This layer can be, for example, an oil, wax, membrane (e.g., semi-permeable membrane), or the like. The well can be sealed at any point, for example, after addition of the support or bead, after addition of the reagents, or after addition of either of these components. The sealing of the well can be useful for a variety of purposes, including preventing escape of beads or loaded reagents from the well, permitting select delivery of certain reagents (e.g., via the use of a semi-permeable membrane), for storage of the well prior to or following further processing, etc.

Once sealed, the well may be subjected to conditions for further processing of a cell (or cells) in the well. For instance, reagents in the well may allow further processing of the cell, e.g., cell lysis, as further described herein. Alternatively, the well (or wells such as those of a well-based array) comprising the cell (or cells) may be subjected to freeze-thaw cycling to process the cell (or cells), e.g., cell lysis. The well containing the cell may be subjected to freezing temperatures (e.g., 0° C., below 0° C., −5° C., −10° C., −15° C., −20° C., −25° C., −30° C., −35° C., −40° C., −45° C., −50° C., −55° C., −60° C., −65° C., −70° C., −80° C., or −85° C.). Freezing may be performed in a suitable manner, e.g., sub-zero freezer or a dry ice/ethanol bath. Following an initial freezing, the well (or wells) comprising the cell (or cells) may be subjected to freeze-thaw cycles to lyse the cell (or cells). In one embodiment, the initially frozen well (or wells) are thawed to a temperature above freezing (e.g., 4° C. or above, 8° C. or above, 12° C. or above, 16° C. or above, 20° C. or above, room temperature, or 25° C. or above). In another embodiment, the freezing is performed for less than 10 minutes (e.g., 5 minutes or 7 minutes) followed by thawing at room temperature for less than 10 minutes (e.g., 5 minutes or 7 minutes). This freeze-thaw cycle may be repeated a number of times, e.g., 2, 3, 4 or more times, to obtain lysis of the cell (or cells) in the well (or wells). In one embodiment, the freezing, thawing and/or freeze/thaw cycling is performed in the absence of a lysis buffer. Additional disclosure related to freeze-thaw cycling is provided in WO2019165181A1, which is incorporated herein by reference in its entirety.

A well can include free reagents and/or reagents encapsulated in, or otherwise coupled to or associated with, supports (e.g., beads), or droplets. In some embodiments, any of the reagents described in this disclosure can be encapsulated in, or otherwise coupled to, a support (e.g., a bead) or a droplet, with any chemicals, particles, and elements suitable for sample processing reactions involving biomolecules, such as, but not limited to, nucleic acid molecules and proteins. For example, a bead or droplet used in a sample preparation reaction for DNA sequencing can include one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to: buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, oligonucleotides, nucleotides, deoxyribonucleotide triphosphates (dNTPs), dideoxyribonucleotide triphosphates (ddNTPs), DNA, RNA, peptide polynucleotides, complementary DNA (cDNA), double stranded DNA (dsDNA), single stranded DNA (ssDNA), plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA, polymerase, ligase, restriction enzymes, proteases, nucleases, protease inhibitors, nuclease inhibitors, chelating agents, reducing agents, oxidizing agents, fluorophores, probes, chromophores, dyes, organics, emulsifiers, surfactants, stabilizers, polymers, water, small molecules, pharmaceuticals, radioactive molecules, preservatives, antibiotics, aptamers, and pharmaceutical drug compounds. As described herein, one or more reagents in the well can be used to perform one or more reactions, including but not limited to: cell lysis, cell fixation, permeabilization, nucleic acid reactions, e.g., nucleic acid extension reactions, amplification, reverse transcription, transposase reactions (e.g., tagmentation), etc.

The wells disclosed herein can be provided as a part of a kit. For example, a kit can include instructions for use, a microwell array or device, and reagents (e.g., beads). The kit can include any useful reagents for performing the processes described herein, e.g., nucleic acid reactions, barcoding of nucleic acid molecules, sample processing (e.g., for cell lysis, fixation, and/or permeabilization).

In some cases, a well includes a support (e.g., a bead) or droplet that includes a set of reagents that has a similar attribute, for example, a set of enzymes, a set of minerals, a set of oligonucleotides, a mixture of different barcode molecules, or a mixture of identical barcode molecules. In other cases, a support (e.g., a bead) or droplet includes a heterogeneous mixture of reagents. In some cases, the heterogeneous mixture of reagents can include all components necessary to perform a reaction. In some cases, such mixture can include all components necessary to perform a reaction, except for 1, 2, 3, 4, 5, or more components necessary to perform a reaction. In some cases, such additional components are contained within, or otherwise coupled to, a different support (e.g., a bead) or droplet, or within a solution within a partition (e.g., microwell) of the system.

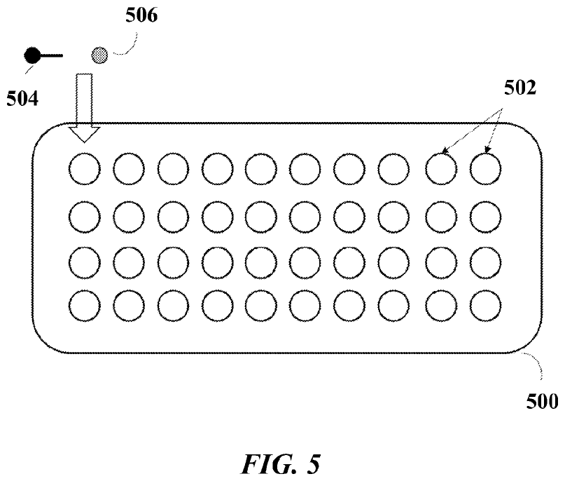

A non-limiting example of a microwell array in accordance with some embodiments of the disclosure is schematically presented in FIG. 5. In this example, the array can be contained within a substrate 500. The substrate 500 includes a plurality of wells 502. The wells 502 can be of any size or shape, and the spacing between the wells, the number of wells per substrate, as well as the density of the wells on the substrate 500 can be modified, depending on the particular application. In one such example application, a sample molecule 506, which can include a cell or cellular components (e.g., nucleic acid molecules) is co-partitioned with a bead 504, which can include a nucleic acid barcode molecule coupled thereto. The wells 502 can be loaded using gravity or other loading technique (e.g., centrifugation, liquid handler, acoustic loading, optoelectronic, etc.). In some instances, at least one of the wells 502 contains a single sample molecule 506 (e.g., cell) and a single bead 504.

Reagents can be loaded into a well either sequentially or concurrently. In some cases, reagents are introduced to the device either before or after a particular operation. In some cases, reagents (which can be provided, in certain instances, in supports (e.g., beads) or droplets) are introduced sequentially such that different reactions or operations occur at different steps. The reagents (or supports (e.g., beads) or droplets) can also be loaded at operations interspersed with a reaction or operation step. For example, supports (e.g., beads) (or droplets) including reagents for fragmenting polynucleotides (e.g., restriction enzymes) and/or other enzymes (e.g., transposases, ligases, polymerases, etc.) can be loaded into the well or plurality of wells, followed by loading of supports (e.g., beads) or droplets, including reagents for attaching nucleic acid barcode molecules to a sample nucleic acid molecule. Reagents can be provided concurrently or sequentially with a sample, e.g., a cell or cellular components (e.g., organelles, proteins, nucleic acid molecules, carbohydrates, lipids, etc.). Accordingly, use of wells can be useful in performing multi-step operations or reactions.

As described elsewhere herein, the nucleic acid barcode molecules and other reagents can be contained within a support (e.g., a bead such as a gel bead) or droplet. These supports or droplets can be loaded into a partition (e.g., a microwell) before, after, or concurrently with the loading of a cell, such that each cell is contacted with a different support (e.g., bead), or droplet. This technique can be used to attach a unique nucleic acid barcode molecule to nucleic acid molecules obtained from each cell. Alternatively or in addition, the sample nucleic acid molecules can be attached to a support. For example, the partition (e.g., microwell) can include a bead which has coupled thereto a plurality of nucleic acid barcode molecules. The sample nucleic acid molecules, or derivatives thereof, can couple or attach to the nucleic acid barcode molecules attached on the support. The resulting barcoded nucleic acid molecules can then be removed from the partition, and in some instances, pooled and sequenced. In such cases, the nucleic acid barcode sequences can be used to trace the origin of the sample nucleic acid molecule. For example, polynucleotides with identical barcodes can be determined to originate from the same cell or partition, while polynucleotides with different barcodes can be determined to originate from different cells or partitions.

The samples or reagents can be loaded in the wells or microwells using a variety of approaches. For example, the samples (e.g., a cell, cell bead, or cellular component) or reagents (as described herein) can be loaded into the well or microwell using an external force, e.g., gravitational force, electrical force, magnetic force, or using mechanisms to drive the sample or reagents into the well, for example, via pressure-driven flow, centrifugation, optoelectronics, acoustic loading, electrokinetic pumping, vacuum, capillary flow, etc. In certain cases, a fluid handling system can be used to load the samples or reagents into the well. The loading of the samples or reagents can follow a Poissonian distribution or a non-Poissonian distribution, e.g., super Poisson or sub-Poisson. The geometry, spacing between wells, density, and size of the microwells can be modified to accommodate a useful sample or reagent distribution; for example, the size and spacing of the microwells can be adjusted such that the sample or reagents can be distributed in a super-Poissonian fashion.

In one non-limiting example, the microwell array or plate includes pairs of microwells, in which each pair of microwells is configured to hold a droplet (e.g., including a single cell) and a single bead (such as those described herein, which can, in some instances, also be encapsulated in a droplet). The droplet and the bead (or droplet containing the bead) can be loaded simultaneously or sequentially, and the droplet and the bead can be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets including different reagents and/or samples, which are merged upon contact or upon application of a stimulus. In such instances, the droplet of one microwell of the pair can include reagents that can react with an agent in the droplet of the other microwell of the pair. For example, one droplet can include reagents that are configured to release the nucleic acid barcode molecules of a bead contained in another droplet, located in the adjacent microwell. Upon merging of the droplets, the nucleic acid barcode molecules can be released from the bead into the partition (e.g., the microwell or microwell pair that are in contact), and further processing can be performed (e.g., barcoding, nucleic acid reactions, etc.). In cases where intact or live cells are loaded in the microwells, one of the droplets can include lysis reagents for lysing the cell upon droplet merging.

In some embodiments, a droplet or support can be partitioned into a well. The droplets can be selected or subjected to pre-processing prior to loading into a well. For instance, the droplets can include cells, and only certain droplets, such as those containing a single cell (or at least one cell), can be selected for use in loading of the wells. Such a pre-selection process can be useful in efficient loading of single cells, such as to obtain a non-Poissonian distribution, or to pre-filter cells for a selected characteristic prior to further partitioning in the wells. Additionally, the technique can be useful in obtaining or preventing cell doublet or multiplet formation prior to or during loading of the microwell.

In some embodiments, the wells can include nucleic acid barcode molecules attached thereto. The nucleic acid barcode molecules can be attached to a surface of the well (e.g., a wall of the well). The nucleic acid barcode molecules may be attached to a droplet or bead that has been partitioned into the well. The nucleic acid barcode molecule (e.g., a partition barcode sequence) of one well can differ from the nucleic acid barcode molecule of another well, which can permit identification of the contents contained with a single partition or well. In some embodiments, the nucleic acid barcode molecule can include a spatial barcode sequence that can identify a spatial coordinate of a well, such as within the well array or well plate. In some embodiments, the nucleic acid barcode molecule can include a unique molecular identifier for individual molecule identification. In some instances, the nucleic acid barcode molecules can be configured to attach to or capture a nucleic acid molecule within a sample or cell distributed in the well. For example, the nucleic acid barcode molecules can include a capture sequence that can be used to capture or hybridize to a nucleic acid molecule (e.g., RNA, DNA) within the sample. In some embodiments, the nucleic acid barcode molecules can be releasable from the microwell. In some instances, the nucleic acid barcode molecules may be releasable from the bead or droplet. For example, the nucleic acid barcode molecules can include a chemical cross-linker which can be cleaved upon application of a stimulus (e.g., photo-, magnetic, chemical, biological, stimulus). The released nucleic acid barcode molecules, which can be hybridized or configured to hybridize to a sample nucleic acid molecule, can be collected and pooled for further processing, which can include nucleic acid processing (e.g., amplification, extension, reverse transcription, etc.) and/or characterization (e.g., sequencing). In some instances nucleic acid barcode molecules attached to a bead or droplet in a well may be hybridized to sample nucleic acid molecules, and the bead with the sample nucleic acid molecules hybridized thereto may be collected and pooled for further processing, which can include nucleic acid processing (e.g., amplification, extension, reverse transcription, etc.) and/or characterization (e.g., sequencing). In such cases, the unique partition barcode sequences can be used to identify the cell or partition from which a nucleic acid molecule originated.

Characterization of samples within a well can be performed. Such characterization can include, in non-limiting examples, imaging of the sample (e.g., cell, cell bead, or cellular components) or derivatives thereof. Characterization techniques such as microscopy or imaging can be useful in measuring sample profiles in fixed spatial locations. For example, when cells are partitioned, optionally with beads, imaging of each microwell and the contents contained therein can provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, etc. In some instances, imaging can be used to characterize live cells in the wells, including, but not limited to: dynamic live-cell tracking, cell-cell interactions (when two or more cells are co-partitioned), cell proliferation, etc. Alternatively or in addition to, imaging can be used to characterize a quantity of amplification products in the well.

In operation, a well can be loaded with a sample and reagents, simultaneously or sequentially. When cells or cell beads are loaded, the well can be subjected to washing, e.g., to remove excess cells from the well, microwell array, or plate. Similarly, washing can be performed to remove excess beads or other reagents from the well, microwell array, or plate. In the instances where live cells are used, the cells can be lysed in the individual partitions to release the intracellular components or cellular analytes. Alternatively, the cells can be fixed or permeabilized in the individual partitions. The intracellular components or cellular analytes can couple to a support, e.g., on a surface of the microwell, on a solid support (e.g., bead), or they can be collected for further downstream processing. For example, after cell lysis, the intracellular components or cellular analytes can be transferred to individual droplets or other partitions for barcoding. Alternatively, or in addition, the intracellular components or cellular analytes (e.g., nucleic acid molecules) can couple to a bead including a nucleic acid barcode molecule; subsequently, the bead can be collected and further processed, e.g., subjected to nucleic acid reaction such as reverse transcription, amplification, or extension, and the nucleic acid molecules thereon can be further characterized, e.g., via sequencing. Alternatively, or in addition, the intracellular components or cellular analytes can be barcoded in the well (e.g., using a bead including nucleic acid barcode molecules that are releasable or on a surface of the microwell including nucleic acid barcode molecules). The barcoded nucleic acid molecules or analytes can be further processed in the well, or the barcoded nucleic acid molecules or analytes can be collected from the individual partitions and subjected to further processing outside the partition. Further processing can include nucleic acid processing (e.g., performing an amplification, extension) or characterization (e.g., fluorescence monitoring of amplified molecules, sequencing). At any suitable or useful step, the well (or microwell array or plate) can be sealed (e.g., using an oil, membrane, wax, etc.), which enables storage of the assay or selective introduction of additional reagents.

### Reagents

In accordance with certain aspects, biological particles can be partitioned along with lysis reagents in order to release the contents of the biological particles within the partition. See, e.g., U.S. Pat. Pub. 2018/0216162 (now U.S. Pat. No. 10,428,326), U.S. Pat. Pub. 2019/0100632 (now U.S. Pat. No. 10,590,244), and U.S. Pat. Pub. 2019/0233878. Biological particles (e.g., cells, cell beads, cell nuclei, organelles, and the like) can be partitioned together with nucleic acid barcode molecules and the nucleic acid molecules of or derived from the biological particle (e.g., mRNA, cDNA, gDNA, etc.) can be barcoded as described elsewhere herein. In some embodiments, biological particles are co-partitioned with barcode carrying beads (e.g., gel beads) and the nucleic acid molecules of or derived from the biological particle are barcoded as described elsewhere herein. In such cases, the lysis agents can be contacted with the biological particle suspension concurrently with, or immediately prior to, the introduction of the biological particles into the partitioning junction/droplet generation zone (e.g., junction 210), such as through an additional channel or channels upstream of the channel junction. In accordance with other aspects, additionally or alternatively, biological particles can be partitioned along with other reagents, as will be described further below.

Beneficially, when lysis reagents and biological particles are co-partitioned, the lysis reagents can facilitate the release of the contents of the biological particles within the partition. The contents released in a partition can remain discrete from the contents of other partitions.

As will be appreciated, the channel segments described herein can be coupled to any of a variety of different fluid sources or receiving components, including reservoirs, tubing, manifolds, or fluidic components of other systems. As will be appreciated, the microfluidic channel structures can have other geometries and/or configurations. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment can be controlled to control the partitioning of the different elements into droplets. Fluid can be directed flow along one or more channels or reservoirs via one or more fluid flow units. A fluid flow unit can include compressors (e.g., providing positive pressure), pumps (e.g., providing negative pressure), actuators, and the like to control flow of the fluid. Fluid can also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents can additionally or alternatively be co-partitioned with the biological particles to cause the release of the biological particle's contents into the partitions. For example, in some cases, surfactant-based lysis solutions can be used to lyse cells (e.g., immune effector cells or labelled engineered cells), although these can be less desirable for emulsion based systems where the surfactants can interfere with stable emulsions. In some cases, lysis solutions can include non-ionic surfactants such as, for example, TritonX-100 and Tween 20. In some cases, lysis solutions can include ionic surfactants such as, for example, sarcosyl and sodium dodecyl sulfate (SDS). Electroporation, thermal, acoustic or mechanical cellular disruption can also be used in certain cases, e.g., non-emulsion based partitioning such as encapsulation of biological particles that can be in addition to or in place of droplet partitioning, where any pore size of the encapsulate is sufficiently small to retain nucleic acid fragments of a given size, following cellular disruption.

Alternatively or in addition to the lysis agents co-partitioned with the biological particles (e.g., immune effector cells or labelled engineered cells) described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles (e.g., immune effector cells or labelled engineered cells), the biological particles can be exposed to an appropriate stimulus to release the biological particles or their contents from a co-partitioned support (e.g., bead). For example, in some cases, a chemical stimulus can be co-partitioned along with an encapsulated biological particle to allow for the degradation of the support (e.g., bead such as a gel bead) and release of the cell or its contents into the larger partition. In some cases, this stimulus can be the same as the stimulus described elsewhere herein for release of nucleic acid molecules (e.g., oligonucleotides) from their respective support (e.g., bead). In alternative aspects, this can be a different and non-overlapping stimulus, in order to allow an encapsulated biological particle to be released into a partition at a different time from the release of nucleic acid molecules into the same partition.

Additional reagents can also be co-partitioned with the biological particles (e.g., immune effector cells or labelled engineered cells), such as endonucleases to fragment a biological particle's DNA, DNA polymerase enzymes and dNTPs used to amplify the biological particle's nucleic acid fragments and to attach the barcode molecular tags to the amplified fragments. Other enzymes can be co-partitioned include, without limitation, polymerase, transposase, ligase, proteinase K, DNAse, etc. Additional reagents can also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides can include a hybridization region and a template region. The hybridization region can include any sequence capable of hybridizing to the target. In some cases, as previously described, the hybridization region includes a series of G bases to complement the overhanging C bases at the 3′ end of a cDNA molecule. The series of G bases can include 1 G base, 2 G bases, 3 G bases, 4 G bases, 5 G bases or more than 5 G bases. The template sequence can include any sequence to be incorporated into the cDNA. In some cases, the template region includes at least 1 (e.g., at least 2, 3, 4, 5 or more) tag sequences and/or functional sequences. Switch oligos can include deoxyribonucleic acids; ribonucleic acids; modified nucleic acids including 2-Aminopurine, 2,6-Diaminopurine (2-Amino-dA), inverted dT, 5-Methyl dC, 2′-deoxyInosine, Super T (5-hydroxybutyl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

In some cases, the length of a switch oligo can be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo can be at most about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

Once the contents of the cells (e.g., immune effector cells or labelled engineered cells) are released into their respective partitions, the macromolecular components (e.g., macromolecular constituents of biological particles, such as RNA, DNA, proteins, or secreted antibodies or antigen-binding fragments thereof) contained therein can be further processed within the partitions. In accordance with the methods and systems described herein, the macromolecular component contents of individual biological particles (e.g., immune effector cells or labelled engineered cells) can be provided with unique identifiers such that, upon characterization of those macromolecular components they can be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles.

In some aspects, this is performed by co-partitioning the individual biological particle (e.g., immune effector cells or labelled engineered cells) or groups of biological particles (e.g., immune effector cells or labelled engineered cells) with the unique identifiers, such as described above (with reference to FIGS. 1 and 2). In some aspects, the unique identifiers are provided in the form of nucleic acid molecules (e.g., oligonucleotides) that include nucleic acid barcode sequences that can be attached to or otherwise associated with the nucleic acid contents of individual biological particle, or to other components of the biological particle, and particularly to fragments of those nucleic acids. The nucleic acid molecules are partitioned such that as between nucleic acid molecules in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences can be present.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence can be about 6, 7, 8, 9, 10, 11, 12, 13, 14, 15, 16, 17, 18, 19, 20 nucleotides or longer. In some cases, the length of a barcode sequence can be at least about 6, 7, 8, 9, 10, 11, 12, 13, 14, 15, 16, 17, 18, 19, 20 nucleotides or longer. In some cases, the length of a barcode sequence can be at most about 6, 7, 8, 9, 10, 11, 12, 13, 14, 15, 16, 17, 18, 19, 20 nucleotides or shorter. These nucleotides can be completely contiguous, i.e., in a single stretch of adjacent nucleotides, or they can be separated into two or more separate subsequences that are separated by 1 or more nucleotides. In some cases, separated barcode subsequences can be from about 4 to about 16 nucleotides in length. In some cases, the barcode subsequence can be about 4, 5, 6, 7, 8, 9, 10, 11, 12, 13, 14, 15, 16 nucleotides or longer. In some cases, the barcode subsequence can be at least about 4, 5, 6, 7, 8, 9, 10, 11, 12, 13, 14, 15, 16 nucleotides or longer. In some cases, the barcode subsequence can be at most about 4, 5, 6, 7, 8, 9, 10, 11, 12, 13, 14, 15, 16 nucleotides or shorter.

The co-partitioned nucleic acid molecules can also include other functional sequences useful in the processing of the nucleic acids from the co-partitioned biological particles (e.g., immune effector cells or labelled engineered cells). These sequences include, e.g., targeted or random/universal amplification primer sequences for amplifying the genomic DNA from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides can also be employed, including, e.g., coalescence of two or more droplets, where one droplet contains oligonucleotides, or microdispensing of oligonucleotides into partitions, e.g., droplets within microfluidic systems.

In an example, supports, such as beads, are provided that each include large numbers of the above described barcoded nucleic acid molecules (e.g., barcoded oligonucleotides) releasably attached to the beads, where all of the nucleic acid molecules attached to a particular bead will include the same nucleic acid barcode sequence, but where a large number of diverse barcode sequences are represented across the population of beads used. In some embodiments, hydrogel beads, e.g., including polyacrylamide polymer matrices, are used as a solid support and delivery vehicle for the nucleic acid molecules into the partitions, as they are capable of carrying large numbers of nucleic acid molecules, and can be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each bead can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set. In some embodiments, such different barcode sequences can be associated with a given bead.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

In some cases, it may be desirable to incorporate multiple different barcodes within a given partition, either attached to a single or multiple beads within the partition. For example, in some cases, a mixed, but known set of barcode sequences can provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus can be a photo-stimulus, e.g., through cleavage of a photo-labile linkage that releases the nucleic acid molecules. In other cases, a thermal stimulus can be used, where elevation of the temperature of the beads environment will result in cleavage of a linkage or other release of the nucleic acid molecules from the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles, and can be degraded for release of the attached nucleic acid molecules through exposure to a reducing agent, such as DTT.

In some embodiments, the method of the disclosure includes generating a plurality of barcoded nucleic acid molecules in the partition that comprises one or more barcode sequences or complements thereof, which identify said antigen-binding molecule as having phagocytotic, opsonophagocytotic activity and/or trogocytotic activity. In some embodiments, the plurality of barcoded nucleic acid molecules comprises a first barcoded nucleic acid molecule comprising the first barcode sequence (i.e., the barcode sequence identifying the antigen-binding molecule) or a complement thereof, and the partition barcode sequence or a complement thereof. In some embodiments, the plurality of barcoded nucleic acid molecules comprises a second barcoded nucleic acid molecule comprising the second barcode sequence (i.e., the barcode sequence identifying the antigen) or a complement thereof, and a partition barcode sequence or a complement thereof. In some embodiments, the plurality of barcoded nucleic acid molecules comprises a third barcoded nucleic acid molecule comprising the third barcode sequence (i.e., the barcode sequence identifying the anti-opsonin antibody) or a complement thereof, and the partition barcode sequence or a complement thereof.

In some embodiments, the plurality of barcoded nucleic acid molecules comprise an additional barcoded nucleic acid molecule comprising a sequence corresponding to a messenger ribonucleic acid (mRNA) molecule encoding for an immune receptor from the immune effector cell that contains the phagocytosed complex (i.e., the first immune effector cell).

The method of the disclosure may further comprise quantifying the sorted cells. The quantification may comprise labeling the separated cells and sorting them. Further, the method can further include isolating the phagocytosed complex from the separated and sorted cells. In some embodiments, the quantification is conducted by sequencing the plurality of barcoded nucleic acid molecules in the partition and analyzing the barcode sequences as described herein. In some embodiments, the barcode sequences include the first, second, and/or third barcode sequences associated with the antigen-binding molecule, the antigen, and/or the anti-opsonin antibody, respectively. In some embodiments, the barcode sequences include the UMI.

For instance, Table 2 illustrates some examples on the identification of the antigen-binding molecule by barcodes. In example 1, the antigen (Ag) and the antibody (Ab) are contacted with each other and form a complex. The complex is subsequently phagocytosed by a phagocytic cell. Thus, the barcodes associated with the antigen and the antibody can be found in the phagocytotic cell. See, for example, the illustration of FIGS. 10A-10C. In example 2, an opsonin and a barcoded anti-opsonin antibody are further added to the system. The opsonized antigen-antibody complex is then engulfed by the phagocytotic cell. Thus, the barcodes associated with the antigen, the antibody, and the anti-opsonin antibody (e.g., an anti-complement antibody) can be found in the phagocytotic cell. See, for example, the illustration of FIGS. 11A-11B. In examples 3 and 4, no antibody-dependent phagocytosis occurs. Thus, no barcodes associated with the antibody can be found in the phagocytotic cell. In example 3, the antigen is opsonized and a barcoded anti-opsonin antibody is further added. The opsonized antigen is subsequently engulfed by the phagocytotic cells. Thus, only the barcode associated with the anti-opsonin antibody (e.g., an anti-complement antibody) can be found in the phagocytotic cell. In contrast, in example 4, no anti-opsonin antibody is added, although the antigen may be opsonized. Thus, no barcodes associated with any of the antigen, the antibody, and/or the anti-opsonin antibody (e.g., an anti-complement antibody) are found in the phagocytotic cell.

TABLE 2
Identification of The
Antigen-Binding Molecule By Barcodes.

| | Ab | Ab | Ag | Ab | Ag | Opsonin |
|---|---|---|---|---|---|---|
| Ex- | binds | phago- | opson- | barcode | barcode | barcode |
| ample | Ag | cytosed | ized | in cell | in cell | in cell |
| 1 | x | x | | x | x | |
| 2 | x | x | x | x | x | x |
| 3 | x | | x | | | x |
| 4 | x | | x | | | |

In some embodiments, the method further comprises comparing the number of partitioned immune effector cells that have ingested the complex and/or at least one opsonin (e.g., complement components) to a reference number quantified for a plurality of reference cells. In some embodiments, the method further comprises comparing the percentage of partitioned immune effector cells that have ingested the complex and/or the at least one complement component to a reference percentage quantified for the plurality of reference effector cells.

A reference effector cell can be a positive reference effector cell or a negative reference effector cell. In some embodiments, a reference effector cell can be an immune effector cell that has been contacted with an antigen coated with neutravidin. In some embodiments, a reference effector cell can be an immune effector cell that has been contacted with an antigen coated with avidin derivative, streptavidin derivative, or streptactin. In some embodiments, a reference effector cell can be an immune effector cell that has been contacted with a negative control antigen-binding molecule having or suspected of having little or no opsonophagocytotic or trogocytotic effects. In some embodiments, a reference effector cell can be an immune effector cell that has been contacted with a positive control antigen-binding molecule having or suspected of having opsonophagocytotic or trogocytotic effects. In some embodiments, a reference effector cell can be an immune effector cell that has been contacted with the complex comprising the antigen bound to the antigen-binding molecule, and has been further contacted with an Fc blocking reagent. In some embodiments, the Fc blocking reagent prevents antigen and antibody uptake.

In some embodiments, an at least about 5% to an at least about 50% increase in the percentage of partitioned immune effector cells that have ingested the complex and the reference percentage characterizes the antigen-binding molecule as having an opsonophagocytotic or trogocytotic activity. In some embodiments, an at least about 10% to an at least about 40% increase in the percentage of partitioned immune effector cells that have ingested the complex and the reference percentage characterizes the antigen-binding molecule as having an opsonophagocytotic or trogocytotic activity. In some embodiments, an at least about 15% to an at least about 30% increase in the percentage of partitioned immune effector cells that have ingested the complex and the reference percentage characterizes the antigen-binding molecule as having an opsonophagocytotic or trogocytotic activity. In some embodiments, an at least about 20% increase in the percentage of partitioned immune effector cells that have ingested the complex and the reference percentage characterizes the antigen-binding molecule as having an opsonophagocytotic or trogocytotic activity. In some embodiments, an at least about 15% increase in the percentage of partitioned immune effector cells that have ingested the complex and the reference percentage characterizes the antigen-binding molecule as having an opsonophagocytotic or trogocytotic activity. In certain embodiments, an at least about 10% increase in the percentage of partitioned immune effector cells that have ingested the complex and the reference percentage characterizes the antigen-binding molecule as having an opsonophagocytotic or trogocytotic activity.

In some embodiments, an at least about 5% to an at least about 50% increase in the percentage of partitioned immune effector cells that have ingested the complex as compared to the reference percentage characterizes the antigen-binding molecule as having an opsonophagocytotic or trogocytotic activity. In some embodiments, an at least about 10% to an at least about 40% increase in the percentage of partitioned immune effector cells that have ingested the complex as compared to the reference percentage characterizes the antigen-binding molecule as having an opsonophagocytotic or trogocytotic activity. In some embodiments, an at least about 15% to an at least about 30% increase in the percentage of partitioned immune effector cells that have ingested the complex as compared to the reference percentage characterizes the antigen-binding molecule as having an opsonophagocytotic or trogocytotic activity. In some embodiments, an at least about 20% increase in the percentage of partitioned immune effector cells that have ingested the complex as compared to the reference percentage characterizes the antigen-binding molecule as having an opsonophagocytotic or trogocytotic activity. In some embodiments, an at least about 15% increase in the percentage of partitioned immune effector cells that have ingested the complex as compared to the reference percentage characterizes the antigen-binding molecule as having an opsonophagocytotic or trogocytotic activity. In certain embodiments, an at least about 10% increase in the percentage of partitioned immune effector cells that have ingested the complex as compared to the reference percentage characterizes the antigen-binding molecule as having an opsonophagocytotic or trogocytotic activity.

### Sample and Cell Processing

A sample can be derived from any useful source including any subject, such as a human subject. A sample can include material (e.g., one or more cells) from one or more different sources, such as one or more different subjects. Multiple samples, such as multiple samples from a single subject (e.g., multiple samples obtained in the same or different manners from the same or different bodily locations, and/or obtained at the same or different times (e.g., seconds, minutes, hours, days, weeks, months, or years apparat)), or multiple samples from different subjects, can be obtained for analysis as described herein. For example, a first sample can be obtained from a subject at a first time and a second sample can be obtained from the subject at a second time later than the first time. The first time can be before a subject undergoes a treatment regimen or procedure (e.g., to address a disease or condition), and the second time can be during or after the subject undergoes the treatment regimen or procedure. In another example, a first sample can be obtained from a first bodily location or system of a subject (e.g., using a first collection technique) and a second sample can be obtained from a second bodily location or system of the subject (e.g., using a second collection technique), which second bodily location or system can be different than the first bodily location or system. In another example, multiple samples can be obtained from a subject at a same time from the same or different bodily locations. Different samples, such as different samples collected from different bodily locations of a same subject, at different times, from multiple different subjects, and/or using different collection techniques, can undergo the same or different processing (e.g., as described herein). For example, a first sample can undergo a first processing protocol and a second sample can undergo a second processing protocol.

A sample can be a biological sample, such as a cell sample (e.g., as described herein). A sample can include one or more analyte carriers, such as one or more cells and/or cellular constituents, such as one or more cell nuclei. For example, a sample can include a plurality of cells and/or cellular constituents. Components (e.g., cells or cellular constituents, such as cell nuclei) of a sample can be of a single type or a plurality of different types. For example, cells of a sample can include one or more different types of blood cells.

A biological sample can include a plurality of cells having different dimensions and features. In some cases, processing of the biological sample, such as cell separation and sorting (e.g., as described herein), can affect the distribution of dimensions and cellular features included in the sample by depleting cells having certain features and dimensions and/or isolating cells having certain features and dimensions.

A sample may undergo one or more processes in preparation for analysis (e.g., as described herein), including, but not limited to, filtration, selective precipitation, purification, centrifugation, permeabilization, isolation, agitation, heating, and/or other processes. For example, a sample may be filtered to remove a contaminant or other materials. In an example, a filtration process can include the use of microfluidics (e.g., to separate analyte carriers of different sizes, types, charges, or other features).

In an example, a sample including one or more cells can be processed to separate the one or more cells from other materials in the sample (e.g., using centrifugation and/or another process). In some cases, cells and/or cellular constituents of a sample can be processed to separate and/or sort groups of cells and/or cellular constituents, such as to separate and/or sort cells and/or cellular constituents of different types. Examples of cell separation include, but are not limited to, separation of white blood cells or immune cells from other blood cells and components, separation of circulating tumor cells from blood, and separation of bacteria from bodily cells and/or environmental materials. A separation process can include a positive selection process (e.g., targeting of a cell type of interest for retention for subsequent downstream analysis, such as by use of a monoclonal antibody that targets a surface marker of the cell type of interest), a negative selection process (e.g., removal of one or more cell types and retention of one or more other cell types of interest), and/or a depletion process (e.g., removal of a single cell type from a sample, such as removal of red blood cells from peripheral blood mononuclear cells).

Separation of one or more different types of cells can include, for example, centrifugation, filtration, microfluidic-based sorting, flow cytometry, fluorescence-activated cell sorting (FACS), magnetic-activated cell sorting (MACS), buoyancy-activated cell sorting (BACS), or any other useful method. For example, a flow cytometry method can be used to detect cells and/or cellular constituents based on a parameter such as a size, morphology, or protein expression. Flow cytometry-based cell sorting can include injecting a sample into a sheath fluid that conveys the cells and/or cellular constituents of the sample into a measurement region one at a time. In the measurement region, a light source such as a laser can interrogate the cells and/or cellular constituents and scattered light and/or fluorescence can be detected and converted into digital signals. A nozzle system (e.g., a vibrating nozzle system) can be used to generate droplets (e.g., aqueous droplets) including individual cells and/or cellular constituents. Droplets including cells and/or cellular constituents of interest (e.g., as determined via optical detection) can be labeled with an electric charge (e.g., using an electrical charging ring), which charge can be used to separate such droplets from droplets including other cells and/or cellular constituents. For example, FACS can include labeling cells and/or cellular constituents with fluorescent markers (e.g., using internal and/or external biomarkers). Cells and/or cellular constituents can then be measured and identified one by one and sorted based on the emitted fluorescence of the marker or absence thereof. MACS can use micro- or nano-scale magnetic particles to bind to cells and/or cellular constituents (e.g., via an antibody interaction with cell surface markers) to facilitate magnetic isolation of cells and/or cellular constituents of interest from other components of a sample (e.g., using a column-based analysis). BACS can use microbubbles (e.g., glass microbubbles) labeled with antibodies to target cells of interest. Cells and/or cellular components coupled to microbubbles can float to a surface of a solution, thereby separating target cells and/or cellular components from other components of a sample. Cell separation techniques can be used to enrich for populations of cells of interest (e.g., prior to partitioning, as described herein). For example, a sample including a plurality of cells including a plurality of cells of a given type can be subjected to a positive separation process. The plurality of cells of the given type can be labeled with a fluorescent marker (e.g., based on an expressed cell surface marker or another marker) and subjected to a FACS process to separate these cells from other cells of the plurality of cells. The selected cells can then be subjected to subsequent partition-based analysis (e.g., as described herein) or other downstream analysis. The fluorescent marker can be removed prior to such analysis or can be retained. The fluorescent marker can include an identifying feature, such as a nucleic acid barcode sequence and/or unique molecular identifier.

In another example, a first sample including a first plurality of cells including a first plurality of cells of a given type (e.g., immune cells expressing a particular marker or combination of markers) and a second sample including a second plurality of cells including a second plurality of cells of the given type can be subjected to a positive separation process. The first and second samples can be collected from the same or different subjects, at the same or different types, from the same or different bodily locations or systems, using the same or different collection techniques. For example, the first sample can be from a first subject and the second sample can be from a second subject different than the first subject. The first plurality of cells of the first sample can be provided a first plurality of fluorescent markers configured to label the first plurality of cells of the given type. The second plurality of cells of the second sample can be provided a second plurality of fluorescent markers configured to label the second plurality of cells of the given type. The first plurality of fluorescent markers can include a first identifying feature, such as a first barcode, while the second plurality of fluorescent markers can include a second identifying feature, such as a second barcode, that is different than the first identifying feature. The first plurality of fluorescent markers and the second plurality of fluorescent markers can fluoresce at the same intensities and over the same range of wavelengths upon excitation with a same excitation source (e.g., light source, such as a laser). The first and second samples can then be combined and subjected to a FACS process to separate cells of the given type from other cells based on the first plurality of fluorescent markers labeling the first plurality of cells of the given type and the second plurality of fluorescent markers labeling the second plurality of cells of the given type. Alternatively, the first and second samples can undergo separate FACS processes and the positively selected cells of the given type from the first sample and the positively selected cells of the given type from the second sample can then be combined for subsequent analysis. The encoded identifying features of the different fluorescent markers can be used to identify cells originating from the first sample and cells originating from the second sample. For example, the first and second identifying features can be configured to interact (e.g., in partitions, as described herein) with nucleic acid barcode molecules (e.g., as described herein) to generate barcoded nucleic acid products detectable using, e.g., nucleic acid sequencing.

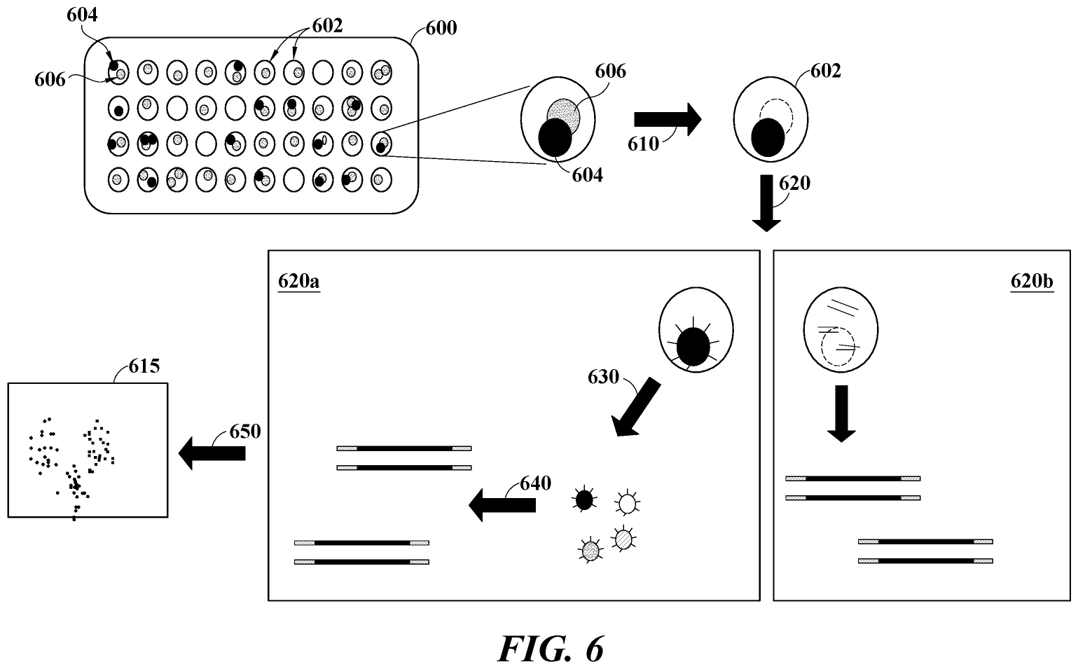

FIG. 6 schematically shows an example workflow for processing nucleic acid molecules within a sample. A substrate 600 including a plurality of microwells 602 can be provided. A sample 606 which can include a cell, cell bead, cellular components or analytes (e.g., proteins and/or nucleic acid molecules) can be co-partitioned, in a plurality of microwells 602, with a plurality of beads 604 including nucleic acid barcode molecules. During a partitioning process, the sample 606 can be processed within the partition. For instance, in the case of live cells, the cell can be subjected to conditions sufficient to lyse the cells and release the analytes contained therein. In process 620, the bead 604 can be further processed. By way of example, processes 620a and 620b schematically illustrate different workflows, depending on the properties of the bead 604.

In 620a, the bead includes nucleic acid barcode molecules that are attached thereto, and sample nucleic acid molecules (e.g., RNA, DNA) can attach, e.g., via hybridization of ligation, to the nucleic acid barcode molecules. Such attachment can occur on the bead. In process 630, the beads 604 from multiple wells 602 can be collected and pooled. Further processing can be performed in process 640. For example, one or more nucleic acid reactions can be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences can be appended to each end of the nucleic acid molecule. In process 650, further characterization, such as sequencing can be performed to generate sequencing reads. The sequencing reads can yield information on individual cells or populations of cells, which can be represented visually or graphically, e.g., in a plot.

In 620b, the bead includes nucleic acid barcode molecules that are releasably attached thereto, as described below. The bead can degrade or otherwise release the nucleic acid barcode molecules into the well 602; the nucleic acid barcode molecules can then be used to barcode nucleic acid molecules within the well 602. Further processing can be performed either inside the partition or outside the partition. For example, one or more nucleic acid reactions can be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences can be appended to each end of the nucleic acid molecule. In process 650, further characterization, such as sequencing can be performed to generate sequencing reads. The sequencing reads can yield information on individual cells or populations of cells, which can be represented visually or graphically, e.g., in a plot.

### Sequencing

A plethora of different approaches, systems, and techniques for nucleic acid sequencing, including next-generation sequencing (NGS) methods, can be used to determine the nucleic acid sequences. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based singleplex methods, emulsion PCR), and/or isothermal amplification.

Non-limiting examples of nucleic acid sequencing methods include Maxam-Gilbert sequencing and chain-termination methods, de novo sequencing methods including shotgun sequencing and bridge PCR, next-generation methods including Polony sequencing, 454 pyrosequencing, Illumina sequencing, SOLiD™ sequencing, Ion Torrent semiconductor sequencing, HeliS cope single molecule sequencing, and SMRT® sequencing.

Other examples of methods for sequencing genetic material include, but are not limited to, DNA hybridization methods, restriction enzyme digestion methods, Sanger sequencing methods, ligation methods, and microarray methods. Additional examples of sequencing methods that can be used include targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, co-amplification at lower denaturation temperature-PCR (COLD-PCR), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, Solexa Genome Analyzer sequencing, MS-PET sequencing, whole transcriptome sequencing, and any combinations thereof.

Sequence analysis of the nucleic acid molecules can be direct or indirect. Thus, the sequence analysis substrate (which can be viewed as the molecule which is subjected to the sequence analysis step or process) can be a barcoded nucleic acid molecule or it can be a molecule which is derived therefrom (e.g., a complement thereof). Thus, for example, in the sequence analysis step of a sequencing reaction, the sequencing template can be the barcoded nucleic acid molecule or it can be a molecule derived therefrom. For example, a first and/or second strand DNA molecule can be directly subjected to sequence analysis (e.g., sequencing), i.e., can directly take part in the sequence analysis reaction or process (e.g., the sequencing reaction or sequencing process, or be the molecule which is sequenced or otherwise identified). Alternatively, a barcoded nucleic acid molecule can be subjected to a step of second strand synthesis or amplification before sequence analysis (e.g., sequencing or identification by another technique). The sequence analysis substrate (e.g., template) can thus be an amplicon or a second strand of a barcoded nucleic acid molecule.

In some embodiments, both strands of a double stranded molecule can be subjected to sequence analysis. In some embodiments, single stranded molecules can be sequenced. In some embodiments, all or a part of the nucleic acid sequences can be determined by using a whole transcriptome sequencing technique, which generally involves sequencing the complete complement of transcripts in a sample, at a given time (often referred to as the transcriptome). Whole transcriptome sequencing generally uses high throughput sequencing technologies to sequence the entire transcriptome in order to get information about a sample's (e.g., an immune effector cell or engineered cell provided herein) RNA content. Whole transcriptome sequencing can be done with a variety of platforms for example, the Genome Analyzer (Illumina, Inc., San Diego, Calif.) and the SOLiD™ Sequencing System (Life Technologies, Carlsbad, Calif.). However, any platform useful for whole transcriptome sequencing may be used. The term “RNA-Seq” or “transcriptome sequencing” refers to sequencing performed on RNA (or cDNA) instead of DNA, where generally, the primary goal is to measure expression levels, detect fusion transcripts, alternative splicing, and other genomic alterations that can be better assessed from RNA. RNA-Seq includes whole transcriptome sequencing as well as target specific sequencing.

### Multiplexing Methods

In some embodiments of the disclosure, the methods described herein can be performed in multiplex format. Accordingly, in some embodiments, the present disclosure provides methods and systems for multiplexing, and otherwise increasing throughput of samples for analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of binding to or otherwise coupling to one or more cells or cell features can be used to characterize cells and/or cell features. In some instances, cell features include cell surface features. Cell surface features can include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features can include intracellular analytes, such as proteins, protein modifications (e.g., phosphorylation status or other post-translational modifications), nuclear proteins, nuclear membrane proteins, or any combination thereof. A labelling agent can include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a Darpin, and a protein scaffold, or any combination thereof. The labelling agents can include (e.g., are attached to) a reporter oligonucleotide that is indicative of the cell surface feature to which the binding group binds. For example, the reporter oligonucleotide can include a barcode sequence that permits identification of the labelling agent. For example, a labelling agent that is specific to one type of cell feature (e.g., a first cell surface feature) can have a first reporter oligonucleotide coupled thereto, while a labelling agent that is specific to a different cell feature (e.g., a second cell surface feature) can have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969.

In a particular example, a library of potential cell feature labelling agents can be provided, where the respective cell feature labelling agents are associated with nucleic acid reporter molecules, such that a different reporter oligonucleotide sequence is associated with each labelling agent capable of binding to a specific cell feature. In other aspects, different members of the library can be characterized by the presence of a different oligonucleotide sequence label. For example, an antibody capable of binding to a first protein can have associated with it a first reporter oligonucleotide sequence, while an antibody capable of binding to a second protein can have a different reporter oligonucleotide sequence associated with it. The presence of the particular oligonucleotide sequence can be indicative of the presence of a particular antibody or cell feature which can be recognized or bound by the particular antibody.

Labelling agents capable of binding to or otherwise coupling to one or more cells can be used to characterize a cell as belonging to a particular set of cells. For example, labeling agents can be used to label a sample of cells or a group of cells. In this way, a group of cells can be labeled as different from another group of cells. In an example, a first group of cells can originate from a first sample and a second group of cells can originate from a second sample. Labelling agents can allow the first group and second group to have a different labeling agent (or reporter oligonucleotide associated with the labeling agent). This can, for example, facilitate multiplexing, where cells of the first group and cells of the second group can be labeled separately and then pooled together for downstream analysis. The downstream detection of a label can indicate analytes as belonging to a particular group.

For example, a reporter oligonucleotide can be linked to an antibody or an epitope binding fragment thereof, and labeling a cell can include subjecting the antibody-linked barcode molecule or the epitope binding fragment-linked barcode molecule to conditions suitable for binding the antibody to a molecule present on a surface of the cell. The binding affinity between the antibody or the epitope binding fragment thereof and the molecule present on the surface can be within a desired range to ensure that the antibody or the epitope binding fragment thereof remains bound to the molecule. For example, the binding affinity can be within a desired range to ensure that the antibody or the epitope binding fragment thereof remains bound to the molecule during various sample processing steps, such as partitioning and/or nucleic acid amplification or extension. A dissociation constant (Kd) between the antibody or an epitope binding fragment thereof and the molecule to which it binds can be less than about 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, 10 pM, 9 pM, 8 pM, 7 pM, 6 pM, 5 pM, 4 pM, 3 pM, 2 pM, or 1 pM. For example, the dissociation constant can be less than about 10 μM.

In another example, a reporter oligonucleotide can be coupled to a cell-penetrating peptide (CPP), and labeling cells can include delivering the CPP coupled reporter oligonucleotide into an analyte carrier. Labeling analyte carriers can include delivering the CPP conjugated oligonucleotide into a cell and/or cell bead by the cell-penetrating peptide. A CPP that can be used in the methods provided herein can include at least one non-functional cysteine residue, which can be either free or derivatized to form a disulfide link with an oligonucleotide that has been modified for such linkage. Non-limiting examples of CPPs that can be used in embodiments herein include penetratin, transportan, plsl, TAT(48-60), pVEC, MTS, and MAP. Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells of a cell population. The CPP can be an arginine-rich peptide transporter. The CPP can be Penetratin or the Tat peptide. In another example, a reporter oligonucleotide can be coupled to a fluorophore or dye, and labeling cells can include subjecting the fluorophore-linked barcode molecule to conditions suitable for binding the fluorophore to the surface of the cell. In some instances, fluorophores can interact strongly with lipid bilayers and labeling cells can include subjecting the fluorophore-linked barcode molecule to conditions such that the fluorophore binds to or is inserted into a membrane of the cell. In some cases, the fluorophore is a water-soluble, organic fluorophore. In some instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 maleimide. See, e.g., Hughes L D, et al. PLoS One. 2014 Feb. 4; 9(2):e87649 for a description of organic fluorophores.

A reporter oligonucleotide can be coupled to a lipophilic molecule, and labeling cells can include delivering the nucleic acid barcode molecule to a membrane of a cell or a nuclear membrane by the lipophilic molecule. Lipophilic molecules can associate with and/or insert into lipid membranes such as cell membranes and nuclear membranes. In some cases, the insertion can be reversible. In some cases, the association between the lipophilic molecule and the cell or nuclear membrane can be such that the membrane retains the lipophilic molecule (e.g., and associated components, such as nucleic acid barcode molecules, thereof) during subsequent processing (e.g., partitioning, cell permeabilization, amplification, pooling, etc.). The reporter nucleotide can enter into the intracellular space and/or a cell nucleus. In some embodiments, a reporter oligonucleotide coupled to a lipophilic molecule will remain associated with and/or inserted into lipid membrane (as described herein) via the lipophilic molecule until lysis of the cell occurs, e.g., inside a partition. Exemplary embodiments of lipophilic molecules coupled to reporter oligonucleotides are described in PCT/US2018/064600.

A reporter oligonucleotide can be part of a nucleic acid molecule including any number of functional sequences, as described elsewhere herein, such as a target capture sequence, a random primer sequence, and the like, and coupled to another nucleic acid molecule that is, or is derived from, the analyte.

Prior to partitioning, the cells can be incubated with the library of labelling agents, that can be labelling agents to a broad panel of different cell features, e.g., receptors, proteins, etc., and which include their associated reporter oligonucleotides. Unbound labelling agents can be washed from the cells, and the cells can then be co-partitioned (e.g., into droplets or wells) along with partition-specific barcode oligonucleotides (e.g., attached to a support, such as a bead or gel bead) as described elsewhere herein. As a result, the partitions can include the cell or cells, as well as the bound labelling agents and their known, associated reporter oligonucleotides.

In other instances, e.g., to facilitate sample multiplexing, a labelling agent that is specific to a particular cell feature can have a first plurality of the labelling agent (e.g., an antibody or lipophilic moiety) coupled to a first reporter oligonucleotide and a second plurality of the labelling agent coupled to a second reporter oligonucleotide. For example, the first plurality of the labeling agent and second plurality of the labeling agent can interact with different cells, cell populations or samples, allowing a particular report oligonucleotide to indicate a particular cell population (or cell or sample) and cell feature. In this way, different samples or groups can be independently processed and subsequently combined together for pooled analysis (e.g., partition-based barcoding as described elsewhere herein). See, e.g., U.S. Pat. Pub. 20190323088.

In some embodiments, to facilitate sample multiplexing, individual samples can be stained with lipid tags, such as cholesterol-modified oligonucleotides (CMOs, see, e.g., FIG. 7), anti-calcium channel antibodies, or anti-ACTB antibodies. Non-limiting examples of anti-calcium channel antibodies include anti-KCNN4 antibodies, anti-BK channel beta 3 antibodies, anti-a1B calcium channel antibodies, and anti-CACNA1A antibodies. Examples of anti-ACTB antibodies suitable for the methods of the disclosure include, but are not limited to, mAbGEa, ACTN05, AC-15, 15G5A11/E2, BA3R, and HIFIF35.

As described elsewhere herein, libraries of labelling agents can be associated with a particular cell feature as well as be used to identify analytes as originating from a particular cell population, or sample. Cell populations can be incubated with a plurality of libraries such that a cell or cells include multiple labelling agents. For example, a cell can include coupled thereto a lipophilic labeling agent and an antibody. The lipophilic labeling agent can indicate that the cell is a member of a particular cell sample, whereas the antibody can indicate that the cell includes a particular analyte. In this manner, the reporter oligonucleotides and labelling agents can allow multi-analyte, multiplexed analyses to be performed.

In some instances, these reporter oligonucleotides can include nucleic acid barcode sequences that permit identification of the labelling agent which the reporter oligonucleotide is coupled to. The use of oligonucleotides as the reporter can provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents can be achieved through any of a variety of direct or indirect, covalent or non-covalent associations or attachments. For example, oligonucleotides can be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715. Likewise, protein and peptide biotinylation techniques have been developed and are readily available. See, e.g., U.S. Pat. No. 6,265,552. Furthermore, click reaction chemistry such as a Methyltetrazine-PEG5-NHS Ester reaction, a TCO-PEG4-NHS Ester reaction, or the like, can be used to couple reporter oligonucleotides to labelling agents. Commercially available kits, such as those from Thunderlink and Abcam, and techniques common in the art can be used to couple reporter oligonucleotides to labelling agents as appropriate. In another example, a labelling agent is indirectly (e.g., via hybridization) coupled to a reporter oligonucleotide including a barcode sequence that identifies the label agent. For instance, the labelling agent can be directly coupled (e.g., covalently bound) to a hybridization oligonucleotide that includes a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide can be attached to the labeling agent through a labile bond (e.g., chemically labile, photolabile, thermally labile, etc.) as generally described for releasing molecules from supports elsewhere herein. In some instances, the reporter oligonucleotides described herein can include one or more functional sequences that can be used in subsequent processing, such as an adapter sequence, a unique molecular identifier (UMI) sequence, a sequencer specific flow cell attachment sequence (such as a P5, P7, or partial P5 or P7 sequence), a primer or primer binding sequence, a sequencing primer or primer biding sequence (such as an R1, R2, or partial R1 or R2 sequence).

In some cases, the labelling agent can include a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to an oligonucleotide that is complementary to a sequence of the reporter oligonucleotide, and the oligonucleotide can be allowed to hybridize to the reporter oligonucleotide.

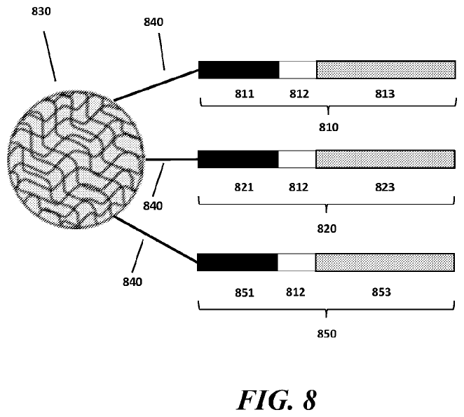

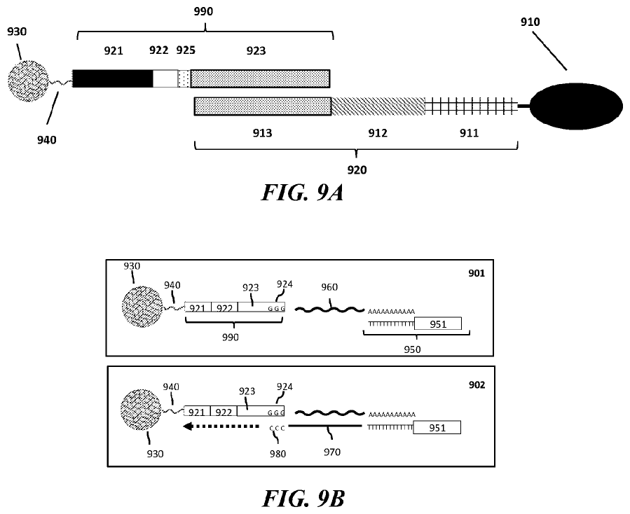

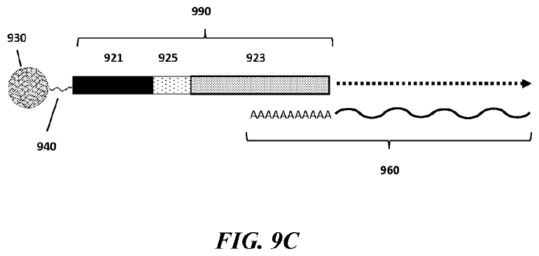

Exemplary barcode molecules attached to a support (e.g., a bead) is shown in FIG. 8. In some embodiments, analysis of multiple analytes (e.g., RNA and one or more analytes using labelling agents described herein) can include nucleic acid barcode molecules as generally depicted in FIG. 8. In some embodiments, nucleic acid barcode molecules 810 and 820 are attached to support 830 via a releasable linkage 840 (e.g., including a labile bond) as described elsewhere herein. Nucleic acid barcode molecule 810 can include functional sequence 811, barcode sequence 812 and capture sequence 813. Nucleic acid barcode molecule 820 can include adapter sequence 821, barcode sequence 812, and capture sequence 823, wherein capture sequence 823 includes a different sequence than capture sequence 813. In some instances, adapter 811 and adapter 821 include the same sequence. In some instances, adapter 811 and adapter 821 include different sequences. Although support 830 is shown including nucleic acid barcode molecules 810 and 820, any suitable number of barcode molecules including common barcode sequence 812 are contemplated herein. For example, in some embodiments, support 830 further includes nucleic acid barcode molecule 850. Nucleic acid barcode molecule 850 can include adapter sequence 851, barcode sequence 812 and capture sequence 853, wherein capture sequence 853 includes a different sequence than capture sequence 813 and 823. In some instances, nucleic acid barcode molecules (e.g., 810, 820, 850) include one or more additional functional sequences, such as a UMI or other sequences described herein. The nucleic acid barcode molecules 810, 820 or 850 can interact with analytes as described elsewhere herein, for example, as depicted in FIGS. 9A-9C.

Referring to FIG. 9A, in an instance where cells are labelled with labeling agents, capture sequence 923 can be complementary to an adapter sequence of a reporter oligonucleotide. Cells can be contacted with one or more reporter oligonucleotide 920 conjugated labelling agents 910 (e.g., polypeptide, antibody, or others described elsewhere herein). In some cases, the cells can be further processed prior to barcoding. For example, such processing steps can include one or more washing and/or cell sorting steps. In some instances, a cell that is bound to labelling agent 910 which is conjugated to oligonucleotide 920 and support 930 (e.g., a bead, such as a gel bead) including nucleic acid barcode molecule 990 is partitioned into a partition amongst a plurality of partitions (e.g., a droplet of a droplet emulsion or a well of a microwell array). In some instances, the partition includes at most a single cell bound to labelling agent 910. In some instances, reporter oligonucleotide 920 conjugated to labelling agent 910 (e.g., polypeptide, an antibody, pMHC molecule such as an MHC multimer, etc.) includes a first functional sequence 911 (e.g., a primer sequence), a barcode sequence 912 that identifies the labelling agent 910 (e.g., the polypeptide, antibody, or peptide of a pMHC molecule or complex), and a capture handle sequence 913. Capture handle sequence 913 can be configured to hybridize to a complementary sequence, such as capture sequence 923 present on a nucleic acid barcode molecule 990 (e.g., partition-specific barcode molecule). In some instances, oligonucleotide 910 includes one or more additional functional sequences, such as those described elsewhere herein.

Barcoded nucleic acid molecules can be generated (e.g., via a nucleic acid reaction, such as nucleic acid extension or ligation) from the constructs described in FIGS. 9A-9C. For example, capture handle sequence 913 can then be hybridized to complementary capture sequence 923 to generate (e.g., via a nucleic acid reaction, such as nucleic acid extension or ligation) a barcoded nucleic acid molecule including cell barcode (e.g., common barcode or partition-specific barcode) sequence 922 (or a reverse complement thereof) and reporter sequence 912 (or a reverse complement thereof). In some embodiments, the nucleic acid barcode molecule 990 (e.g., partition-specific barcode molecule) further includes a UMI (925). Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) can be performed. For example, the workflow can include a workflow as generally depicted in any of FIGS. 9A-9C, or a combination of workflows for an individual analyte, as described elsewhere herein. For example, by using a combination of the workflows as generally depicted in FIGS. 9A-9C, multiple analytes can be analyzed.

In some instances, analysis of an analyte (e.g. a nucleic acid, a polypeptide, a carbohydrate, a lipid, etc.) includes a workflow as generally depicted in FIG. 9A. A nucleic acid barcode molecule 990 can be co-partitioned with the one or more analytes. In some instances, nucleic acid barcode molecule 990 is attached to a support 930 (e.g., a bead, such as a gel bead), such as those described elsewhere herein. For example, nucleic acid barcode molecule 990 can be attached to support 930 via a releasable linkage 940 (e.g., including a labile bond), such as those described elsewhere herein. Nucleic acid barcode molecule 990 can include a functional sequence 921 and optionally include other additional sequences, for example, a barcode sequence 922 (e.g., common barcode, partition-specific barcode, or other functional sequences described elsewhere herein), and/or a UNIT sequence 925. The nucleic acid barcode molecule 990 can include a capture sequence 923 that can be complementary to another nucleic acid sequence, such that it can hybridize to a particular sequence.

For example, capture sequence 923 can include a poly-T sequence and can be used to hybridize to mRNA. Referring to FIG. 9C, in some embodiments, nucleic acid barcode molecule 990 includes capture sequence 923 complementary to a sequence of RNA molecule 960 from a cell. In some instances, capture sequence 923 includes a sequence specific for an RNA molecule. Capture sequence 923 can include a known or targeted sequence or a random sequence. In some instances, a nucleic acid extension reaction can be performed, thereby generating a barcoded nucleic acid product including capture sequence 923, the functional sequence 921, UMI sequence 925, any other functional sequence, and a sequence corresponding to the RNA molecule 960.

In another example, capture sequence 923 can be complementary to an overhang sequence or an adapter sequence that has been appended to an analyte. For example, referring to FIG. 9B, in some embodiments, primer 950 includes a sequence complementary to a sequence of nucleic acid molecule 960 (such as an RNA encoding for a BCR sequence) from an analyte carrier. In some instances, primer 950 includes one or more sequences 951 that are not complementary to RNA molecule 960. Sequence 951 can be a functional sequence as described elsewhere herein, for example, an adapter sequence, a sequencing primer sequence, or a sequence the facilitates coupling to a flow cell of a sequencer. In some instances, primer 950 includes a poly-T sequence. In some instances, primer 950 includes a sequence complementary to a target sequence in an RNA molecule. In some instances, primer 950 includes a sequence complementary to a region of an immune molecule, such as the constant region of a TCR or BCR sequence. Primer 950 is hybridized to nucleic acid molecule 960 and complementary molecule 970 is generated. For example, complementary molecule 970 can be cDNA generated in a reverse transcription reaction. In some instances, an additional sequence can be appended to complementary molecule 970. For example, the reverse transcriptase enzyme can be selected such that several non-templated bases 980 (e.g., a poly-C sequence) are appended to the cDNA. In another example, a terminal transferase can also be used to append the additional sequence. Nucleic acid barcode molecule 990 includes a sequence 924 complementary to the non-templated bases, and the reverse transcriptase performs a template switching reaction onto nucleic acid barcode molecule 990 to generate a barcoded nucleic acid molecule including cell (e.g., partition specific) barcode sequence 922 (or a reverse complement thereof) and a sequence of complementary molecule 970 (or a portion thereof). In some instances, capture sequence 923 includes a sequence complementary to a region of an immune molecule, such as the constant region of a TCR or BCR sequence. Capture sequence 923 is hybridized to nucleic acid molecule 960 and a complementary molecule 970 is generated. For example, complementary molecule 970 can be generated in a reverse transcription reaction generating a barcoded nucleic acid molecule including cell barcode (e.g., common barcode or partition-specific barcode) sequence 922 (or a reverse complement thereof) and a sequence of complementary molecule 970 (or a portion thereof). Additional methods and compositions suitable for barcoding cDNA generated from mRNA transcripts including those encoding V(D)J regions of an immune cell receptor and/or barcoding methods and composition including a template switch oligonucleotide are described in International Patent Application WO2018/075693, U.S. Patent Publication No. 2018/0105808, U.S. Patent Publication No. 2015/0376609, and U.S. Patent Publication No. 2019/0367969.

### Combinatorial Barcoding

In some instances, barcoding of a nucleic acid molecule may be done using a combinatorial approach. In such instances, one or more nucleic acid molecules (which may be comprised in a cell, e.g., a fixed cell, or cell bead) may be partitioned (e.g., in a first set of partitions, e.g., wells or droplets) with one or more first nucleic acid barcode molecules (optionally coupled to a bead). The first nucleic acid barcode molecules or derivative thereof (e.g., complement, reverse complement) may then be attached to the one or more nucleic acid molecules, thereby generating first barcoded nucleic acid molecules, e.g., using the processes described herein. The first nucleic acid barcode molecules may be partitioned to the first set of partitions such that a nucleic acid barcode molecule, of the first nucleic acid barcode molecules, that is in a partition comprises a barcode sequence that is unique to the partition among the first set of partitions. Each partition may comprise a unique barcode sequence. For example, a set of first nucleic acid barcode molecules partitioned to a first partition in the first set of partitions may each comprise a common barcode sequence that is unique to the first partition among the first set of partitions, and a second set of first nucleic acid barcode molecules partitioned to a second partition in the first set of partitions may each comprise another common barcode sequence that is unique to the second partition among the first set of partitions. Such barcode sequence (unique to the partition) may be useful in determining the cell or partition from which the one or more nucleic acid molecules (or derivatives thereof) originated.

The first barcoded nucleic acid molecules from multiple partitions of the first set of partitions may be pooled and re-partitioned (e.g., in a second set of partitions, e.g., one or more wells or droplets) with one or more second nucleic acid barcode molecules. The second nucleic acid barcode molecules or derivative thereof may then be attached to the first barcoded nucleic acid molecules, thereby generating second barcoded nucleic acid molecules. As with the first nucleic acid barcode molecules during the first round of partitioning, the second nucleic acid barcode molecules may be partitioned to the second set of partitions such that a nucleic acid barcode molecule, of the second nucleic acid barcode molecules, that is in a partition comprises a barcode sequence that is unique to the partition among the second set of partitions. Such barcode sequence may also be useful in determining the cell or partition from which the one or more nucleic acid molecules or first barcoded nucleic acid molecules originated. The second barcoded nucleic acid molecules may thus comprise two barcode sequences (e.g., from the first nucleic acid barcode molecules and the second nucleic acid barcode molecules).

Additional barcode sequences may be attached to the second barcoded nucleic acid molecules by repeating the processes any number of times (e.g., in a split-and-pool approach), thereby combinatorically synthesizing unique barcode sequences to barcode the one or more nucleic acid molecules. For example, combinatorial barcoding may comprise at least 1, 2, 3, 4, 6, 7, 8, 9, 10 or more operations of splitting (e.g., partitioning) and/or pooling (e.g., from the partitions). Additional examples of combinatorial barcoding may also be found in International Patent Publication Nos. WO2019/165318, each of which is herein entirely incorporated by reference for all purposes.

Beneficially, the combinatorial barcode approach may be useful for generating greater barcode diversity, and synthesizing unique barcode sequences on nucleic acid molecules derived from a cell or partition. For example, combinatorial barcoding comprising three operations, each with 100 partitions, may yield up to 10⁶ unique barcode combinations. In some instances, the combinatorial barcode approach may be helpful in determining whether a partition contained only one cell or more than one cell. For instance, the sequences of the first nucleic acid barcode molecule and the second nucleic acid barcode molecule may be used to determine whether a partition comprised more than one cell. For instance, if two nucleic acid molecules comprise different first barcode sequences but the same second barcode sequences, it may be inferred that the second set of partitions comprised two or more cells.

In some instances, combinatorial barcoding may be achieved in the same compartment. For instance, a unique nucleic acid molecule comprising one or more nucleic acid bases may be attached to a nucleic acid molecule (e.g., a sample or target nucleic acid molecule) in successive operations within a partition (e.g., droplet or well) to generate a first barcoded nucleic acid molecule. A second unique nucleic acid molecule comprising one or more nucleic acid bases may be attached to the first barcoded nucleic acid molecule molecule, thereby generating a second barcoded nucleic acid molecule. In some instances, all the reagents for barcoding and generating combinatorially barcoded molecules may be provided in a single reaction mixture, or the reagents may be provided sequentially.

In some instances, cell beads comprising nucleic acid molecules may be barcoded. Methods and systems for barcoding cell beads are further described in PCT/US2018/067356 and U.S. Pat. Pub. No. 2019/0330694, which are hereby incorporated by reference in its entirety.

### Compositions

The present disclosure also provides compositions that include an immune effector cell associated with a complex. The complex comprises an antigen-binding molecule bound to an antigen, and the antigen-binding molecule (i) is exogenous to the immune effector cell and (ii) comprises a first oligonucleotide comprising a first barcode sequence.

The complex can be a phagocytosed complex within the immune effector cell, and/or the antigen can include a second oligonucleotide comprising a second barcode sequence, and/or the antigen can be associated with opsonin deposition, optionally wherein the opsonin deposition comprises complement deposition, and/or the antigen can be conjugated to a support, optionally wherein the support comprises a bead, optionally wherein the bead comprises gel beads, glass beads, magnetic beads, and/or ceramic beads.

In some embodiments, the composition further includes a partition comprising the immune effector cell, optionally where the partition is a droplet or a well, and/or the partition further includes a plurality of nucleic acid barcode molecules, where a first nucleic acid barcode molecule of the plurality of nucleic acid barcode molecules comprises a partition barcode sequence, optionally where the plurality of nucleic acid barcode molecules are attached to a bead, optionally where the bead is a solid bead, a magnetic bead, or a gel bead.

In some embodiments, the immune effector cell of the composition is (i) capable of mediating antibody-dependent cellular phagocytosis (ADCP) and/or (ii) capable of antibody-dependent cellular trogocytosis (ADCT), and/or (iii) a phagocytic cell and/or a trogocytotic cell, optionally wherein the phagocytic cell is selected from a neutrophil, monocyte, macrophage, mast cell, and dendritic cell, optionally where the trogocytotic cell is selected from a B cell, T cell, monocyte, neutrophil, and natural killer cell.

### Kits

In some embodiments, a kit comprises reagents configured to conjugate a first oligonucleotide comprising a first barcode sequence to an antigen binding molecule and instructions for performing the methods described herein. The kit can also include the first oligonucleotide as described herein. The kit can further include a second and/or a third oligonucleotide, and these reagents can be configured to conjugate a second oligonucleotide comprising a second barcode sequence to an antigen capable of binding the antigen binding molecule, and/or to conjugate a third oligonucleotide comprising a third barcode sequence to an anti-opsonin antibody. The anti-opsonin antibody can be included in the kit. As described above, the kit can further comprise a support, wherein the reagents are configured to conjugate the antigen to the support, or wherein the kit further comprises reagents configured to conjugate the antigen to the support. A control antigen that is configured to or expected to not bind the antigen binding molecule may also be present. In some embodiments, the kit also includes a population of immune effector cells.

### Systems

The present disclosure is also directed to a system. The system can include an antigen binding molecule comprising a first oligonucleotide comprising a first barcode sequence; an antigen that binds the antigen binding molecule; and a plurality of nucleic acid barcode molecules, wherein a first nucleic acid barcode molecule of the plurality of nucleic acid barcode molecules comprises a partition barcode sequence.

In some embodiments, the plurality of nucleic acid barcode molecules is attached to a bead, and the partition barcode sequence identifies the bead. The first nucleic acid barcode molecule can include a first capture sequence configured to couple to the first oligonucleotide. In some embodiments, the first oligonucleotide further includes a capture handle sequence configured to couple to the capture sequence of the first nucleic acid barcode molecule.

In some embodiments, the antigen of the system includes a second oligonucleotide comprising a second barcode sequence. The second nucleic acid barcode molecule of the plurality of nucleic acid barcode molecules can comprise the partition barcode sequence and a second capture sequence configured to couple to the second oligonucleotide.

In some embodiments, the first capture sequence and the second capture sequence are identical. Alternatively, in some embodiments the first capture sequence and the second capture sequence are different.

The system of the present disclosure can further comprise an anti-opsonin antibody comprising a third oligonucleotide comprising a third barcode sequence. The third nucleic acid barcode molecule of the plurality of nucleic acid barcode molecules can comprise the partition barcode sequence and a third capture sequence configured to couple to the second oligonucleotide.

In some embodiments, the system includes a fourth nucleic acid barcode molecule of the plurality of nucleic acid barcode molecules that comprises the partition barcode sequence and a fourth capture sequence. The fourth capture sequence is configured to couple to a sequence of the nucleic acid analyte or is a template switch oligonucleotide.

The system can also comprise a plurality of partitions, optionally where the plurality of partitions comprises a plurality of droplets and/or a plurality of wells.

In some embodiments, the system includes an apparatus comprising a microfluidic channel structure configured to generate a plurality of partitions.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

1. A method for identifying opsonophagocytotic activity and/or trogocytotic activity of an antigen-binding molecule, comprising:
a) contacting an antigen with an antigen-binding molecule to create a complex comprising the antigen bound to the antigen-binding molecule, wherein the antigen-binding molecule comprises a first oligonucleotide comprising a first barcode sequence;
b) contacting the complex with a plurality of immune effector cells under conditions sufficient to provide a first immune effector cell comprising the complex as a phagocytosed complex;
c) partitioning the plurality of immune effector cells into a plurality of partitions, wherein a partition of the plurality of partitions comprises (i) the first immune effector cell and (ii) a plurality of nucleic acid barcode molecules wherein a first nucleic acid barcode molecule of the plurality of nucleic acid barcode molecules comprises a partition barcode sequence;
d) in the partition, coupling the first oligonucleotide to the first nucleic acid barcode molecule; and
e) using the first oligonucleotide coupled to the first nucleic acid barcode molecule to generate a first barcoded nucleic acid molecule comprising the first barcode sequence or a complement thereof and the partition barcode sequence or a complement thereof.

2. A method for identifying opsonophagocytotic activity and/or trogocytotic activity of an antigen-binding molecule, comprising:
a) contacting an antigen with an antigen-binding molecule to create a complex comprising the antigen bound to the antigen-binding molecule, wherein the antigen-binding molecule comprises a first oligonucleotide comprising a first barcode sequence;
b) contacting the complex with a plurality of immune effector cells under conditions sufficient to provide a first immune effector cell comprising the complex as a phagocytosed complex;
c) partitioning the plurality of immune effector cells into a plurality of partitions, wherein a partition of the plurality of partitions comprises (i) the first immune effector cell and (ii) a plurality of nucleic acid barcode molecules wherein a first nucleic acid barcode molecule of the plurality of nucleic acid barcode molecules comprises a partition barcode sequence; and
d) in the partition, using the first oligonucleotide and the first nucleic acid barcode molecule to generate a first barcoded nucleic acid molecule comprising the first barcode sequence or a complement thereof and the partition barcode sequence or a complement thereof.

3. The method of claim 1, wherein said antigen comprises a second oligonucleotide comprising a second barcode sequence.

4. The method of claim 3, wherein a second nucleic acid barcode molecule of the plurality of nucleic acid barcode molecules comprises the partition barcode sequence, and wherein the method further comprises using the second oligonucleotide and the second nucleic acid barcode molecule to generate a second barcoded nucleic acid molecule comprising the second barcode sequence or a reverse complement thereof and the partition barcode sequence or a reverse complement thereof.

5. The method of claim 1, wherein said contacting in (b) further comprises conditions sufficient to allow opsonization of said antigen.

6. The method of claim 5, wherein said opsonization of said antigen comprises opsonin deposition of said antigen.

7. The method of claim 6, wherein said opsonin deposition of said antigen comprises complement deposition of said antigen.

8. The method of claim 6, further comprising contacting the plurality of immune effector cells with an anti-opsonin antibody.

9. (canceled)

10. The method of claim 8, wherein the anti-opsonin antibody comprises a third oligonucleotide comprising a third barcode sequence.

11. The method of claim 10, wherein said plurality of barcoded nucleic acid molecules further comprises the third barcode sequence or a complement thereof.

12. The method of claim 10, wherein a third nucleic acid barcode molecule of the plurality of nucleic acid barcode molecules comprises the partition barcode sequence, and wherein the method further comprises using the third oligonucleotide and the third nucleic acid barcode molecule to generate a third barcoded nucleic acid molecule comprising the third barcode sequence or a reverse complement thereof and the partition barcode sequence or a reverse complement thereof.

13. The method of claim 10, wherein the immune effector cell comprises a nucleic acid analyte, and wherein a fourth nucleic acid barcode molecule of the plurality of nucleic acid barcode molecules comprises the partition barcode sequence, and wherein the method further comprises using the nucleic acid analyte and the fourth nucleic acid barcode molecule to generate a fourth barcoded nucleic acid molecule comprising a sequence of the nucleic acid analyte or a reverse complement thereof and the partition barcode sequence or a reverse complement thereof.

14. The method of claim 1, wherein the antigen is presented on the surface of an antigen-presenting cell (APC).

15. The method of claim 1, wherein the antigen is conjugated to a support.

16. The method of claim 15, wherein the support comprises a bead.

17. (canceled)

18. The method of claim 2, wherein the plurality of nucleic acid barcode molecules comprises a partition barcode sequence.

19. The method of claim 1, wherein said plurality of immune effector cells is (i) capable of mediating antibody-dependent cellular phagocytosis (ADCP) and/or (ii) capable of antibody-dependent cellular trogocytosis (ADCT).

20. The method of claim 1, wherein the plurality of immune effector cells comprises a plurality of phagocytotic cells and/or a plurality of trogocytotic cells.

21-22. (canceled)

23. The method of claim 1, further comprising separating the first immune effector cell from a second immune effector cell which does not comprise a phagocytosed complex.

24-26. (canceled)

27. The method of claim 1, further comprising sorting said plurality of immune effector cells prior to said partitioning step.

28-31. (canceled)

32. The method of claim 2, wherein said plurality of barcoded nucleic acid molecules comprises a first barcoded nucleic acid molecule comprising said first barcode sequence or a complement thereof and said partition barcode sequence or a complement thereof.

33. The method of claim 2, wherein the plurality of nucleic acid barcode molecules comprises a partition barcode sequence.

34-37. (canceled)

38. The method of claim 1, further comprising comparing the number of partitioned immune effector cells that have ingested the complex and/or at least one complement components to a reference number quantified for a plurality of reference cells.

39-41. (canceled)

42. The method of claim 1, wherein the partition-specific barcode molecule comprises one or more of the following: a peptide tag, an oligonucleotide barcode, a functional sequence, a common barcode, a UMI, and a reporter capture sequence.

43. (canceled)

44. The method of claim 1, wherein the antigen-binding molecule is conjugated to a reporter oligonucleotide.

45. The method of claim 1, wherein the antigen-binding molecule is conjugated to the first oligonucleotide.

46. The method of claim 44, wherein the reporter oligonucleotide comprises one or more of the following: a reporter capture handle, a reporter sequence, and/or a functional sequence.

47. (canceled)

48. The method of claim 1, further comprising determining a sequence of the first barcoded nucleic acid molecule or a derivative thereof, the second barcoded nucleic acid molecule or a derivative thereof, the third barcoded nucleic acid molecule or a derivative thereof, and/or the fourth barcoded nucleic acid molecule or a derivative thereof.

49. The method of claim 48, comprising (i) using the determined sequence of the first barcoded nucleic acid molecule or a derivative thereof to identify the antigen binding molecule as having been opsonophagocytosed and/or trogocytosed by the first immune effector cell, (ii) using the determined sequence of the second barcoded nucleic acid molecule or a derivative thereof to identify the antigen binding molecule as having bound the antigen, and/or (iii) using the determined sequence of the third barcoded nucleic acid molecule or a derivative thereof to identify the antigen as having been opsonized.

50. A composition, comprising an immune effector cell associated with a complex, the complex comprising an antigen-binding molecule bound to an antigen, wherein the antigen-binding molecule (i) is exogenous to the immune effector cell and (ii) comprises a first oligonucleotide comprising a first barcode sequence.

51-53.

54. A system, comprising:
a) an antigen binding molecule comprising a first oligonucleotide comprising a first barcode sequence;
b) an antigen that binds the antigen binding molecule; and
c) a plurality of nucleic acid barcode molecules, wherein a first nucleic acid barcode molecule of the plurality of nucleic acid barcode molecules comprises a partition barcode sequence.

55-73. (canceled)
